SEQUENCE LISTING (190 sequences)

SEQ ID NO: 1 - protein, 178 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHDTLLLTWSNPYPPDNYLYNHLTYAVNIPADFRIYNVTYLEPSLRIAARARVRAWAQLYNTTWSEWSPSTKW

SEQ ID NO: 2 - protein, 304 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQTLEELSYQALALFSGAQHEKG

SEQ ID NO: 3 - protein, 231 aa, Macaca fascicularis
MGWLCSGLLFPVSCLVLLQVASSGSMKVLQEPTCVSDYMSISTCEWKMGGPTNCSAELRLLYQLVFQSSETHTCVPENNGGVGCVCHLLMDDVVSMDNYTLDLWAGQQLLWKGSFKPSEHVKPRAPGNLTVHTNVSDTVLLTWSNPYPPDNYLYNDLTYAVNIWSENDPAYSRIHNVTYLKPTLRIPASTLKSGISYRARVRAWAQHYNTTWSEWSPSTKWYNSYREPFEQ

SEQ ID NO: 4 - protein, 125 aa, Synthetic construct
EVQLVESGGGLEQPGGSLRLSCAGSGFXFXDYAMTWVRQAPGKGLEWVSSISGSGXXXYYADSVKGRFTISRDNSKNTLYLQMNSLRAEDTAVYYCAXDXXXXXXXPXYXGLDXWGQGTTVTVSS

SEQ ID NO: 5 - protein, 125 aa, Synthetic construct [sequence omitted]

SEQ ID NO: 6 - protein, 125 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCAKDALTIAIRPYYYGLDVWGQGTTVTVSS

SEQ ID NO: 7 - protein, 125 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCARDRLTAAVRPYYYGLDVWGQGTTVTVSS

SEQ ID NO: 8 - protein, 125 aa, Synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSKTIRPYYYGLDVWGQGTTVTVSS

SEQ ID NO: 9 - protein, 125 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCARDRLTITIRPRYYGLDVWGQGTTVTVSS

SEQ ID NO: 10 - protein, 125 aa, Synthetic construct
EVQLVESGGGLEQPGGSLRLSCAGSGFTFSDYAMTWVRQAPGKGLEWVSSISGSGSNTYYADSVKGRFTISRDNSKNTLYLQMNSLRAEDTAVYYCAKDRLTIAIRPYYYGLDVWGQGTTVTVSS

SEQ ID NO: 11 - protein, 125 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTTKVRPEYYGLDVWGQGTTVTVSS

SEQ ID NO: 12 - protein, 125 aa, Synthetic construct
EVQLVESGGGLEQPGGSLRLSCAGSGFTFRDYAMTWVRQAPGKGLEWVSSISGSGSTTYYADSVKGRFTISRDNSKNTLYLQMNSLRAEDTAVYYCAKDRLTASVRPIYVGLDVWGQGTTVTVSS

SEQ ID NO: 13 - protein, 125 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTASVRPNYYGLDVWGQGTTVTVSS

SEQ ID NO: 14 - protein, 125 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTTAVRPSYYGLDVWGQGTTVTVSS

SEQ ID NO: 15 - protein, 125 aa, Synthetic construct
EVQLVESGGGLEQPGGSLRLSCAGSGFTFRDYAMTWVRQAPGKGLEWVSSISGSGDKTYYADSVKGRFTISRDNSKNTLYLQMNSLRAEDTAVYYCARDRLSSSARPVYVGLDVWGQGTTVTVSS

SEQ ID NO: 16 - protein, 125 aa, Synthetic construct
EVQLVESGGGLEQPGGSLRLSCAGSGFTFRDYAMTWVRQAPGKGLEWVSSISGSGTNTYYADSVKGRFTISRDNSKNTLYLQMNSLRAEDTAVYYCAKDRLSVSTRPKYYGLDVWGQGTTVTVSS

SEQ ID NO: 17 - protein, 125 aa, Synthetic construct
EVQLVESGGGLEQPGGSLRLSCAGSGFTFRDYAMTWVRQAPGKGLEWVSSISGSGSNKYYADSVKGRFTISRDNSKNTLYLQMNSLRAEDTAVYYCAKDRLTSRVRPYYYGLDVWGQGTTVTVSS

SEQ ID NO: 18 - protein, 125 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRWTSSVRPVYRGLDIWGQGTTVTVSS

SEQ ID NO: 19 - protein, 125 aa, Synthetic construct
EVQLVESGGGLEQPGGSLRLSCAGSGFTFRDYAMTWVRQAPGKGLEWVSSISGSGSNIYYADSVKGRFTISRDNSKNTLYLQMNSLRAEDTAVYYCAKDRYTKSVRPVYRGLDIWGQGTTVTVSS

SEQ ID NO: 20 - protein, 125 aa, Synthetic construct
EVQLVESGGGLEQPGGSLRLSCAGSGFNFKDYAMTWVRQAPGKGLEWVSSISGSGSNTYYADSVKGRFTISRDNSKNTLYLQMNSLRAEDTAVYYCAKDRWTSSVKPRYRGLDIWGQGTTVTVSS

SEQ ID NO: 21 - protein, 125 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCAKDALSITIRPRYYGLDVWGQGTTVTVSS

SEQ ID NO: 22 - protein, 125 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTAAVRPRYYGLDVWGQGTTVTVSS

SEQ ID NO: 23 - protein, 112 aa, Synthetic construct
DIVMTQSPLSLPVTPGEPASISCRSSQSLXYSXGXNYLDWYLQKSGQSPQLLIYLXSNRASGVPDRFSGSGSGTDFTLKISRVEAEDVGFYYCMQXLXXPYTFGQGTKLEIK

SEQ ID NO: 24 - protein, 112 aa, Synthetic construct [sequence omitted]

SEQ ID NO: 25 - protein, 112 aa, Synthetic construct
DIVMTQSPLSLPVTPGEPASISCRSSQSLDYSYGYNYLDWYLQKSGQSPQLLIYLASNRASGVPDRFSGSGSGTDFTLKISRVEAEDVGFYYCMQSLSTPYTFGQGTKLEIK

SEQ ID NO: 26 - protein, 112 aa, Synthetic construct
DIVMTQSPLSLPVTPGEPASISCRSSQSLDYSSGYNYLDWYLQKSGQSPQLLIYLASNRASGVPDRFSGSGSGTDFTLKISRVEAEDVGFYYCMQSLQTPYTFGQGTKLEIK

SEQ ID NO: 27 - protein, 112 aa, Synthetic construct
DIVMTQSPLSLPVTPGEPASISCRSSQSLLYSTGYNYLDWYLQKSGQSPQLLIYLGSNRASGVPDRFSGSGSGTDFTLKISRVEAEDVGFYYCMQSLSTPYTFGQGTKLEIK

SEQ ID NO: 28 - protein, 112 aa, Synthetic construct
DIVMTQSPLSLPVTPGEPASISCRSSQSLLYSTGYNYLDWYLQKSGQSPQLLIYLASNRASGVPDRFSGSGSGTDFTLKISRVEAEDVGFYYCMQALSTPYTFGQGTKLEIK

SEQ ID NO: 29 - protein, 112 aa, Synthetic construct
DIVMTQSPLSLPVTPGEPASISCRSSQSLDYSIGYNYLDWYLQKSGQSPQLLIYLASNRASGVPDRFSGSGSGTDFTLKISRVEAEDVGFYYCMQALQTPYTFGQGTKLEIK

SEQ ID NO: 30 - protein, 112 aa, Synthetic construct
DIVMTQSPLSLPVTPGEPASISCRSSQSLLYSIGYNYLDWYLQKSGQSPQLLIYLASNRASGVPDRFSGSGSGTDFTLKISRVEAEDVGFYYCMQSLQTPYTFGQGTKLEIK

SEQ ID NO: 31 - protein, 112 aa, Synthetic construct
DIVMTQSPLSLPVTPGEPASISCRSSQSLLYSEGYNYLDWYLQKSGQSPQLLIYLASNRASGVPDRFSGSGSGTDFTLKISRVEAEDVGFYYCMQTLKTPYTFGQGTKLEIK

SEQ ID NO: 32 - protein, 112 aa, Synthetic construct
DIVMTQSPLSLPVTPGEPASISCRSSQSLLYSEGYNYLDWYLQKSGQSPQLLIYLASNRASGVPDRFSGSGSGTDFTLKISRVEAEDVGFYYCMQSLTTPYTFGQGTKLEIK

SEQ ID NO: 33 - protein, 112 aa, Synthetic construct
DIVMTQSPLSLPVTPGEPASISCRSSQSLLYSLGYNYLDWYLQKSGQSPQLLIYLASNRASGVPDRFSGSGSGTDFTLKISRVEAEDVGFYYCMQSLQTPYTFGQGTKLEIK

SEQ ID NO: 34 - protein, 112 aa, Synthetic construct
DIVMTQSPLSLPVTPGEPASISCRSSQSLLYSVGKNYLDWYLQKSGQSPQLLIYLASNRASGVPDRFSGSGSGTDFTLKISRVEAEDVGFYYCMQDLFHPYTFGQGTKLEIK

SEQ ID NO: 35 - protein, 112 aa, Synthetic construct
DIVMTQSPLSLPVTPGEPASISCRSSQSLDYSYGYNYLDWYLQKSGQSPQLLIYLASNRASGVPDRFSGSGSGTDFTLKISRVEAEDVGFYYCMQSLHTPYTFGQGTKLEIK

SEQ ID NO: 36 - protein, 112 aa, Synthetic construct
DIVMTQSPLSLPVTPGEPASISCRSSQSLLYSQGYNYLDWYLQKSGQSPQLLIYLASNRASGVPDRFSGSGSGTDFTLKISRVEAEDVGFYYCMQSLFTPYTFGQGTKLEIK

SEQ ID NO: 37 - protein, 112 aa, Synthetic construct
DIVMTQSPLSLPVTPGEPASISCRSSQSLLYSHGYNYLDWYLQKSGQSPQLLIYLASNRASGVPDRFSGSGSGTDFTLKISRVEAEDVGFYYCMQSLYTPYTFGQGTKLEIK

SEQ ID NO: 38 - protein, 112 aa, Synthetic construct
DIVMTQSPLSLPVTPGEPASISCRSSQSLLYSHGYNYLDWYLQKSGQSPQLLIYLASNRASGVPDRFSGSGSGTDFTLKISRVEAEDVGFYYCMQSLKTPYTFGQGTKLEIK

SEQ ID NO: 39 - protein, 112 aa, Synthetic construct
DIVMTQSPLSLPVTPGEPASISCRSSQSLLYSRGYNYLDWYLQKSGQSPQLLIYLASNRASGVPDRFSGSGSGTDFTLKISRVEAEDVGFYYCMQSLYTPYTFGQGTKLEIK

SEQ ID NO: 40 - protein, 112 aa, Synthetic construct
DIVMTQSPLSLPVTPGEPASISCRSSQSLLYSIGYNYLDWYLQKSGQSPQLLIYLASNRASGVPDRFSGSGSGTDFTLKISRVEAEDVGFYYCMQALQTPYTFGQGTKLEIK

SEQ ID NO: 41 - protein, 112 aa, Synthetic construct
DIVMTQSPLSLPVTPGEPASISCRSSQSLLYSSGYNYLDWYLQKSGQSPQLLIYLASNRASGVPDRFSGSGSGTDFTLKISRVEAEDVGFYYCMQSLQTPYTFGQGTKLEIK

SEQ ID NO: 42 - protein, 8 aa, Synthetic construct
GFXFXDYA

SEQ ID NO: 43 - protein, 8 aa, Synthetic construct
GFTFRDYA

SEQ ID NO: 44 - protein, 8 aa, Synthetic construct
GFTFSDYA

SEQ ID NO: 45 - protein, 8 aa, Synthetic construct
GFNFKDYA

SEQ ID NO: 46 - protein, 8 aa, Synthetic construct
ISGSGXXX

SEQ ID NO: 47 - protein, 8 aa, Synthetic construct
ISGSGGNT

SEQ ID NO: 48 - protein, 8 aa, Synthetic construct
ISGSGSNT

SEQ ID NO: 49 - protein, 8 aa, Synthetic construct
ISGSGSTT

SEQ ID NO: 50 - protein, 8 aa, Synthetic construct
ISGSGDKT

SEQ ID NO: 51 - protein, 8 aa, Synthetic construct
ISGSGTNT

SEQ ID NO: 52 - protein, 8 aa, Synthetic construct
ISGSGSNK

SEQ ID NO: 53 - protein, 8 aa, Synthetic construct
ISGSGSNI

SEQ ID NO: 54 - protein, 17 aa, Synthetic construct
AXDXXXXXXXPXYXGLD

SEQ ID NO: 55 - protein, 17 aa, Synthetic construct
AKDRLSITIRPRYYGLD

SEQ ID NO: 56 - protein, 17 aa, Synthetic construct
AKDALTIAIRPYYYGLD

SEQ ID NO: 57 - protein, 17 aa, Synthetic construct
ARDRLTAAVRPYYYGLD

SEQ ID NO: 58 - protein, 17 aa, Synthetic construct
AKDRLSKTIRPYYYGLD

SEQ ID NO: 59 - protein, 17 aa, Synthetic construct
ARDRLTITIRPRYYGLD

SEQ ID NO: 60 - protein, 17 aa, Synthetic construct
AKDRLTIAIRPYYYGLD

SEQ ID NO: 61 - protein, 17 aa, Synthetic construct
AKDRLTTKVRPEYYGLD

SEQ ID NO: 62 - protein, 17 aa, Synthetic construct
AKDRLTASVRPIYVGLD

SEQ ID NO: 63 - protein, 17 aa, Synthetic construct
AKDRLTASVRPNYYGLD

SEQ ID NO: 64 - protein, 17 aa, Synthetic construct
AKDRLTTAVRPSYYGLD

SEQ ID NO: 65 - protein, 17 aa, Synthetic construct
ARDRLSSSARPVYVGLD

SEQ ID NO: 66 - protein, 17 aa, Synthetic construct
AKDRLSVSTRPKYYGLD

SEQ ID NO: 67 - protein, 17 aa, Synthetic construct
AKDRLTSRVRPYYYGLD

SEQ ID NO: 68 - protein, 17 aa, Synthetic construct
AKDRWTSSVRPVYRGLD

SEQ ID NO: 69 - protein, 17 aa, Synthetic construct
AKDRYTKSVRPVYRGLD

SEQ ID NO: 70 - protein, 17 aa, Synthetic construct
AKDRWTSSVKPRYRGLD

SEQ ID NO: 71 - protein, 11 aa, Synthetic construct
QSLXYSXGXNY

SEQ ID NO: 72 - protein, 11 aa, Synthetic construct
QSLLYSIGYNY

SEQ ID NO: 73 - protein, 11 aa, Synthetic construct
QSLDYSYGYNY

SEQ ID NO: 74 - protein, 11 aa, Synthetic construct
QSLDYSSGYNY

SEQ ID NO: 75 - protein, 11 aa, Synthetic construct
QSLLYSTGYNY

SEQ ID NO: 76 - protein, 11 aa, Synthetic construct
QSLDYSIGYNY

SEQ ID NO: 77 - protein, 11 aa, Synthetic construct
QSLLYSEGYNY

SEQ ID NO: 78 - protein, 11 aa, Synthetic construct
QSLLYSLGYNY

SEQ ID NO: 79 - protein, 11 aa, Synthetic construct
QSLLYSVGKNY

SEQ ID NO: 80 - protein, 11 aa, Synthetic construct
QSLLYSQGYNY

SEQ ID NO: 81 - protein, 11 aa, Synthetic construct
QSLLYSHGYNY

SEQ ID NO: 82 - protein, 11 aa, Synthetic construct
QSLLYSRGYNY

SEQ ID NO: 83-85 - skipped (3 entries)

SEQ ID NO: 86 - protein, 8 aa, Synthetic construct
MQXLXXPY

SEQ ID NO: 87 - protein, 8 aa, Synthetic construct
MQALQTPY

SEQ ID NO: 88 - protein, 8 aa, Synthetic construct
MQSLSTPY

SEQ ID NO: 89 - protein, 8 aa, Synthetic construct
MQSLQTPY

SEQ ID NO: 90 - protein, 8 aa, Synthetic construct
MQALSTPY

SEQ ID NO: 91 - protein, 8 aa, Synthetic construct
MQTLKTPY

SEQ ID NO: 92 - protein, 8 aa, Synthetic construct
MQSLTTPY

SEQ ID NO: 93 - protein, 8 aa, Synthetic construct
MQDLFHPY

SEQ ID NO: 94 - protein, 8 aa, Synthetic construct
MQSLHTPY

SEQ ID NO: 95 - protein, 8 aa, Synthetic construct
MQSLFTPY

SEQ ID NO: 96 - protein, 8 aa, Synthetic construct
MQSLYTPY

SEQ ID NO: 97 - protein, 8 aa, Synthetic construct
MQSLKTPY

SEQ ID NO: 98 - protein, 327 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 327 aa, Synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 100 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 107 aa, Homo sapiens [sequence omitted]

SEQ ID NO: 103 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 104 - protein, 452 aa, Synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 452 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCAKDALTIAIRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCARDRLTAAVRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 107 - protein, 452 aa, Synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSKTIRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 452 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCARDRLTITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 452 aa, Synthetic construct
EVQLVESGGGLEQPGGSLRLSCAGSGFTFSDYAMTWVRQAPGKGLEWVSSISGSGSNTYYADSVKGRFTISRDNSKNTLYLQMNSLRAEDTAVYYCAKDRLTIAIRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTTKVRPEYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 111 - protein, 452 aa, Synthetic construct
EVQLVESGGGLEQPGGSLRLSCAGSGFTFRDYAMTWVRQAPGKGLEWVSSISGSGSTTYYADSVKGRFTISRDNSKNTLYLQMNSLRAEDTAVYYCAKDRLTASVRPIYVGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 112 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTASVRPNYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTTAVRPSYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 452 aa, Synthetic construct
EVQLVESGGGLEQPGGSLRLSCAGSGFTFRDYAMTWVRQAPGKGLEWVSSISGSGDKTYYADSVKGRFTISRDNSKNTLYLQMNSLRAEDTAVYYCARDRLSSSARPVYVGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 452 aa, Synthetic construct
EVQLVESGGGLEQPGGSLRLSCAGSGFTFRDYAMTWVRQAPGKGLEWVSSISGSGTNTYYADSVKGRFTISRDNSKNTLYLQMNSLRAEDTAVYYCAKDRLSVSTRPKYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 452 aa, Synthetic construct
EVQLVESGGGLEQPGGSLRLSCAGSGFTFRDYAMTWVRQAPGKGLEWVSSISGSGSNKYYADSVKGRFTISRDNSKNTLYLQMNSLRAEDTAVYYCAKDRLTSRVRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRWTSSVRPVYRGLDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 118 - protein, 452 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCAKDALTIAIRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 119 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCARDRLTAAVRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 120 - protein, 452 aa, Synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSKTIRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 121 - protein, 452 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCARDRLTITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 122 - protein, 452 aa, Synthetic construct
EVQLVESGGGLEQPGGSLRLSCAGSGFTFSDYAMTWVRQAPGKGLEWVSSISGSGSNTYYADSVKGRFTISRDNSKNTLYLQMNSLRAEDTAVYYCAKDRLTIAIRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 123 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTTKVRPEYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 124 - protein, 452 aa, Synthetic construct
EVQLVESGGGLEQPGGSLRLSCAGSGFTFRDYAMTWVRQAPGKGLEWVSSISGSGSTTYYADSVKGRFTISRDNSKNTLYLQMNSLRAEDTAVYYCAKDRLTASVRPIYVGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 125 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTASVRPNYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 126 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTTAVRPSYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 127 - protein, 452 aa, Synthetic construct
EVQLVESGGGLEQPGGSLRLSCAGSGFTFRDYAMTWVRQAPGKGLEWVSSISGSGDKTYYADSVKGRFTISRDNSKNTLYLQMNSLRAEDTAVYYCARDRLSSSARPVYVGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 128 - protein, 452 aa, Synthetic construct
EVQLVESGGGLEQPGGSLRLSCAGSGFTFRDYAMTWVRQAPGKGLEWVSSISGSGTNTYYADSVKGRFTISRDNSKNTLYLQMNSLRAEDTAVYYCAKDRLSVSTRPKYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 129 - protein, 452 aa, Synthetic construct
EVQLVESGGGLEQPGGSLRLSCAGSGFTFRDYAMTWVRQAPGKGLEWVSSISGSGSNKYYADSVKGRFTISRDNSKNTLYLQMNSLRAEDTAVYYCAKDRLTSRVRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 130 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRWTSSVRPVYRGLDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 131 - protein, 452 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCAKDALTIAIRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 132 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCARDRLTAAVRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 133 - protein, 452 aa, Synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSKTIRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 134 - protein, 452 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCARDRLTITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 135 - protein, 452 aa, Synthetic construct
EVQLVESGGGLEQPGGSLRLSCAGSGFTFSDYAMTWVRQAPGKGLEWVSSISGSGSNTYYADSVKGRFTISRDNSKNTLYLQMNSLRAEDTAVYYCAKDRLTIAIRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 136 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTTKVRPEYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 137 - protein, 452 aa, Synthetic construct
EVQLVESGGGLEQPGGSLRLSCAGSGFTFRDYAMTWVRQAPGKGLEWVSSISGSGSTTYYADSVKGRFTISRDNSKNTLYLQMNSLRAEDTAVYYCAKDRLTASVRPIYVGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 138 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTASVRPNYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 139 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTTAVRPSYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 140 - protein, 452 aa, Synthetic construct
EVQLVESGGGLEQPGGSLRLSCAGSGFTFRDYAMTWVRQAPGKGLEWVSSISGSGDKTYYADSVKGRFTISRDNSKNTLYLQMNSLRAEDTAVYYCARDRLSSSARPVYVGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 141 - protein, 452 aa, Synthetic construct
EVQLVESGGGLEQPGGSLRLSCAGSGFTFRDYAMTWVRQAPGKGLEWVSSISGSGTNTYYADSVKGRFTISRDNSKNTLYLQMNSLRAEDTAVYYCAKDRLSVSTRPKYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 142 - protein, 452 aa, Synthetic construct
EVQLVESGGGLEQPGGSLRLSCAGSGFTFRDYAMTWVRQAPGKGLEWVSSISGSGSNKYYADSVKGRFTISRDNSKNTLYLQMNSLRAEDTAVYYCAKDRLTSRVRPYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 143 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRWTSSVRPVYRGLDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 144 - protein, 452 aa, Synthetic construct
EVQLVESGGGLEQPGGSLRLSCAGSGFTFRDYAMTWVRQAPGKGLEWVSSISGSGSNIYYADSVKGRFTISRDNSKNTLYLQMNSLRAEDTAVYYCAKDRYTKSVRPVYRGLDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 145 - protein, 452 aa, Synthetic construct
EVQLVESGGGLEQPGGSLRLSCAGSGFNFKDYAMTWVRQAPGKGLEWVSSISGSGSNTYYADSVKGRFTISRDNSKNTLYLQMNSLRAEDTAVYYCAKDRWTSSVKPRYRGLDIWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 146 - protein, 452 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCAKDALSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 452 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTAAVRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 148 - protein, 455 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCAKDALTIAIRPYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 455 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCARDRLTAAVRPYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 455 aa, Synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSKTIRPYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 455 aa, Synthetic construct
EVQLVESGGGLEQPGGSLRLSCAGSGFTFSDYAMTWVRQAPGKGLEWVSSISGSGGNTYYADSVKGRFTISRDNSKNTLYLQMNSLRAEDTAVYYCARDRLTITIRPRYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 455 aa, Synthetic construct
EVQLVESGGGLEQPGGSLRLSCAGSGFTFSDYAMTWVRQAPGKGLEWVSSISGSGSNTYYADSVKGRFTISRDNSKNTLYLQMNSLRAEDTAVYYCAKDRLTIAIRPYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 455 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTTKVRPEYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 455 aa, Synthetic construct
EVQLVESGGGLEQPGGSLRLSCAGSGFTFRDYAMTWVRQAPGKGLEWVSSISGSGSTTYYADSVKGRFTISRDNSKNTLYLQMNSLRAEDTAVYYCAKDRLTASVRPIYVGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 455 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTASVRPNYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 455 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRLTTAVRPSYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 455 aa, Synthetic construct
EVQLVESGGGLEQPGGSLRLSCAGSGFTFRDYAMTWVRQAPGKGLEWVSSISGSGDKTYYADSVKGRFTISRDNSKNTLYLQMNSLRAEDTAVYYCARDRLSSSARPVYVGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 455 aa, Synthetic construct
EVQLVESGGGLEQPGGSLRLSCAGSGFTFRDYAMTWVRQAPGKGLEWVSSISGSGTNTYYADSVKGRFTISRDNSKNTLYLQMNSLRAEDTAVYYCAKDRLSVSTRPKYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 455 aa, Synthetic construct
EVQLVESGGGLEQPGGSLRLSCAGSGFTFRDYAMTWVRQAPGKGLEWVSSISGSGSNKYYADSVKGRFTISRDNSKNTLYLQMNSLRAEDTAVYYCAKDRLTSRVRPYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 160 - protein, 455 aa, Synthetic construct
EVQLVESGGGLEQPGGSLRLSCAGSGFTFRDYAMTWVRQAPGKGLEWVSSISGSGSNTYYADSVKGRFTISRDNSKNTLYLQMNSLRAEDTAVYYCAKDRWTSSVRPVYRGLDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 219 aa, Synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 219 aa, Synthetic construct
DIVMTQSPLSLPVTPGEPASISCRSSQSLDYSYGYNYLDWYLQKSGQSPQLLIYLASNRASGVPDRFSGSGSGTDFTLKISRVEAEDVGFYYCMQSLSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 219 aa, Synthetic construct
DIVMTQSPLSLPVTPGEPASISCRSSQSLDYSSGYNYLDWYLQKSGQSPQLLIYLASNRASGVPDRFSGSGSGTDFTLKISRVEAEDVGFYYCMQSLQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 219 aa, Synthetic construct
DIVMTQSPLSLPVTPGEPASISCRSSQSLLYSTGYNYLDWYLQKSGQSPQLLIYLGSNRASGVPDRFSGSGSGTDFTLKISRVEAEDVGFYYCMQSLSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 219 aa, Synthetic construct
DIVMTQSPLSLPVTPGEPASISCRSSQSLLYSTGYNYLDWYLQKSGQSPQLLIYLASNRASGVPDRFSGSGSGTDFTLKISRVEAEDVGFYYCMQALSTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 166 - protein, 219 aa, Synthetic construct
DIVMTQSPLSLPVTPGEPASISCRSSQSLDYSIGYNYLDWYLQKSGQSPQLLIYLASNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 167 - protein, 219 aa, Synthetic construct
DIVMTQSPLSLPVTPGEPASISCRSSQSLLYSIGYNYLDWYLQKSGQSPQLLIYLASNRASGVPDRFSGSGSGTDFTLKISRVEAEDVGFYYCMQSLQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 168 - protein, 219 aa, Synthetic construct
DIVMTQSPLSLPVTPGEPASISCRSSQSLLYSEGYNYLDWYLQKSGQSPQLLIYLASNRASGVPDRFSGSGSGTDFTLKISRVEAEDVGFYYCMQTLKTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 169 - protein, 219 aa, Synthetic construct
DIVMTQSPLSLPVTPGEPASISCRSSQSLLYSEGYNYLDWYLQKSGQSPQLLIYLASNRASGVPDRFSGSGSGTDFTLKISRVEAEDVGFYYCMQSLTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 219 aa, Synthetic construct
DIVMTQSPLSLPVTPGEPASISCRSSQSLLYSLGYNYLDWYLQKSGQSPQLLIYLASNRASGVPDRFSGSGSGTDFTLKISRVEAEDVGFYYCMQSLQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 219 aa, Synthetic construct
DIVMTQSPLSLPVTPGEPASISCRSSQSLLYSVGKNYLDWYLQKSGQSPQLLIYLASNRASGVPDRFSGSGSGTDFTLKISRVEAEDVGFYYCMQDLFHPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 172 - protein, 219 aa, Synthetic construct
DIVMTQSPLSLPVTPGEPASISCRSSQSLDYSYGYNYLDWYLQKSGQSPQLLIYLASNRASGVPDRFSGSGSGTDFTLKISRVEAEDVGFYYCMQSLHTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 219 aa, Synthetic construct
DIVMTQSPLSLPVTPGEPASISCRSSQSLLYSQGYNYLDWYLQKSGQSPQLLIYLASNRASGVPDRFSGSGSGTDFTLKISRVEAEDVGFYYCMQSLFTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 174 - protein, 219 aa, Synthetic construct
DIVMTQSPLSLPVTPGEPASISCRSSQSLLYSHGYNYLDWYLQKSGQSPQLLIYLASNRASGVPDRFSGSGSGTDFTLKISRVEAEDVGFYYCMQSLYTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 219 aa, Synthetic construct
DIVMTQSPLSLPVTPGEPASISCRSSQSLLYSHGYNYLDWYLQKSGQSPQLLIYLASNRASGVPDRFSGSGSGTDFTLKISRVEAEDVGFYYCMQSLKTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 176 - protein, 219 aa, Synthetic construct
DIVMTQSPLSLPVTPGEPASISCRSSQSLLYSRGYNYLDWYLQKSGQSPQLLIYLASNRASGVPDRFSGSGSGTDFTLKISRVEAEDVGFYYCMQSLYTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 219 aa, Synthetic construct
DIVMTQSPLSLPVTPGEPASISCRSSQSLLYSIGYNYLDWYLQKSGQSPQLLIYLASNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 219 aa, Synthetic construct
DIVMTQSPLSLPVTPGEPASISCRSSQSLLYSSGYNYLDWYLQKSGQSPQLLIYLASNRASGVPDRFSGSGSGTDFTLKISRVEAEDVGFYYCMQSLQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 179 - protein, 42 aa, Synthetic construct
QLDSSYNLASMQSLQTYFTFRDYSSNTARDRLTAAVRYYLDV

SEQ ID NO: 180 - protein, 42 aa, Synthetic construct
QLLSTYNLASMQALSTYFTFSDYSGNTARDRLTITIRRYLDV

SEQ ID NO: 181 - protein, 42 aa, Synthetic construct
QLDSIYNLASMQALQTYFTFSDYSSNTAKDRLTIAIRYYLDV

SEQ ID NO: 182 - protein, 42 aa, Synthetic construct
QLLSIYNLASMQSLQTYFTFRDYSSNTAKDRLTTKVREYLDV

SEQ ID NO: 183 - protein, 42 aa, Synthetic construct
QLLSEYNLASMQTLKTYFTFRDYSSTTAKDRLTASVRIVLDV

SEQ ID NO: 184 - protein, 42 aa, Synthetic construct
QLLSEYNLASMQSLTTYFTFRDYSSNTAKDRLTASVRNYLDV

SEQ ID NO: 185 - protein, 42 aa, Synthetic construct
QLLSLYNLASMQSLQTYFTFRDYSSNTAKDRLTTAVRSYLDV

SEQ ID NO: 186 - protein, 42 aa, Synthetic construct
QLLSVKNLASMQDLFHYFTFRDYSDKTARDRLSSSARVVLDV

SEQ ID NO: 187 - protein, 42 aa, Synthetic construct
QLDSYYNLASMQSLHTYFTFRDYSTNTAKDRLSVSTRKYLDV

SEQ ID NO: 188 - protein, 42 aa, Synthetic construct
QLLSQYNLASMQSLFTYFTFRDYSSNKAKDRLTSRVRYYLDV

SEQ ID NO: 189 - protein, 42 aa, Synthetic construct
QLLSHYNLASMQSLYTYFTFRDYSSNTAKDRWTSSVRVRLDI

SEQ ID NO: 190 - protein, 42 aa, Synthetic construct
QLLSSYNLASMQSLQTYFTFRDYSSNTAKDRLTAAVRRYLDV

(19) United States
(12) Patent Application Publication
(10) Pub. No.: US 2026/0008857 A1
(43) Pub. Date: Jan. 8, 2026

(54) Antigen Binding Molecules Targeting Interleukine-4 Receptor Subunit Alpha (IL-4Ra)

(71) Applicant: Flagship Pioneering Innovations VI, LLC, Cambridge, MA (US)

(72) Inventors: Antonios O. Aliprantis, Natick, MA (US); Zachary Kohl Costello, Cambridge, MA (US); Anthony John Coyle, Boston, MA (US); Lana Dinic, Newton, MA (US); Kristen Park Hopson, Lincoln, MA (US); Ahmed Ismail, Somerville, MA (US); Brinda Monian, McLean, VA (US); Ryan Terrell Phennicie, Andover, MA (US); Alexis Hiram Ramos, Wilbraham, MA (US); Karin Alma Frieda Reif, San Francisco, CA (US); Adam Reid Root, Newbury, MA (US)

(21) Appl. No.: 19/238,280

(22) Filed: Jun. 13, 2025

Related U.S. Application Data
(60) Provisional application No. 63/659,671, filed on Jun. 13, 2024.

(51) Int. Cl. C07K 16/28; A61P 37/06; G16B 15/30

(52) U.S. Cl. CPC C07K 16/2866; A61P 37/06; G16B 15/30; C07K 2317/24; C07K 2317/565; C07K 2317/567; C07K 2317/92

(57) ABSTRACT

The disclosure provides, in various embodiments, polypeptides (e.g., antibodies and antigen binding fragments thereof) that bind to IL-4 receptor subunit alpha (IL-4Rα). The disclosure also provides, in various embodiments, fusion proteins comprising one or more of polypeptides, polynucleotides encoding polypeptides, vectors and host cells suitable for expressing polypeptides, and methods for treating inflammatory conditions (e.g., atopic dermatitis).

### RELATED APPLICATION

This application claims the benefit of U.S. Provisional Application No. 63/659,671, filed on Jun. 13, 2024. The entire teachings of the above applications are incorporated herein by reference.

### INCORPORATION BY REFERENCE OF MATERIAL IN XML

This application incorporates by reference the Sequence Listing contained in the following extensible Markup Language (XML) file being submitted concurrently herewith:
a) File name: 57081079-001 Sequence Listing.xml; created Jun. 13, 2025, 199,802 Bytes in size.

### INCORPORATION BY REFERENCE OF MATERIAL IN ASCII FILES

This Application incorporates by reference the Computer Program Listing contained in the following ASCII files being submitted concurrently herewith:
a) File name: score_concise.txt; created Jun. 13, 2025, 4,901 Bytes in size.
b) File name: score.txt; created Jun. 13, 2025, 5,250 Bytes in size.
c) File name: fit_model.txt; created Jun. 13, 2025, 4,613,144 Bytes in size.

### BACKGROUND

The interleukin 4 (IL-4)/interleukin 13 (IL-13) axis has been implicated in numerous inflammatory conditions, including asthma, eczema (atopic dermatitis), food allergy, prurigo nodularis, chronic rhinosinusitis with nasal polyps, keloids, eosinophilic esophagitis, prostate cancer, chronic urticaria, bullous pemphigoid, localized scleroderma, alopecia areata, ulcerative colitis, aspirin-exacerbated respiratory disease, metastatic non-small cell lung cancer, and Netherton syndrome. According to the World Health Organization, in 2019 alone, an estimated 262 million people globally were affected by asthma, resulting in 455,000 deaths.

IL-4 receptor subunit alpha (IL-4Rα) mediates the activity of IL-4 and IL-13, two key proximal type 2 cytokines involved in type 2 immunity against mucosal pathogens and allergens (Gandhi et al., Targeting key proximal drivers of type 2 inflammation in disease, Nat. Rev. Drug Discov. 15:35-50 (2016)). Despite development of candidate anti-IL-4Rα therapies, only one has been FDA-approved. Accordingly, a widespread need exists for additional therapeutics that can target the IL-4Rα-mediated pathogenic pathways driving many immunological/inflammatory diseases (Nur Husna et al., IL-4/IL-13 axis as therapeutic targets in allergic rhinitis and asthma, PeerJ. 10:13444 (2022); Shi et al., Involvement of IL-4, IL-13 and Their Receptors in Pancreatic Cancer, Int. J. Mol. Sci. 22 (6): 2998 (2021)).

### SUMMARY

There is a widespread need to develop novel therapeutic agents targeting the IL-4/IL-13 axis. The disclosure provides such therapeutics.

The disclosure provided herein is based, in part, on the discovery that polypeptides disclosed herein bind to interleukin-4 receptor alpha (IL-4Rα). Accordingly, the disclosure generally relates to compositions (e.g., polypeptides, pharmaceutical compositions) and methods that are useful for modulating (e.g., reducing) IL-4Rα-mediated immune activity.

The disclosure also provides, among other things, polypeptides (e.g., polypeptides that specifically binds an IL-4Rα) comprising:
a V_{H} amino acid sequence comprising a heavy chain complementarity determining region 1 (HCDR1), a heavy chain complementarity determining region 2 (HCDR2) and a heavy chain complementarity determining region 3 (HCDR3) that are substantially similar to a HCDR1, a HCDR2 and a HCDR3, respectively, of any one of SEQ ID NOs: 6-20; and
a V_{L} amino acid sequence comprising a light chain complementarity determining region 1 (LCDR1), a light chain complementarity determining region 2 (LCDR2) and a light chain complementarity determining region 3 (LCDR3) that are substantially similar to a LCDR1, a LCDR2 and a LCDR3, respectively, of any one of SEQ ID NOs: 25-39.

In some embodiments, a polypeptide comprises a HCDR1, HCDR2 and HCDR3, and a LCDR1, LCDR2 and LCDR3, of an antibody comprising a V_{H}/V_{L} combination selected from:
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c); or
SEQ ID NO:20 and SEQ ID NO:39 (AB-15c).

The disclosure provides, among other things, polypeptides (e.g., polypeptides that specifically binds an IL-4Rα) comprising a paratope that is substantially similar to a paratope of an antibody comprising an immunoglobulin heavy chain variable domain (V_{H})/an immunoglobulin light chain variable domain (V_{L}) combination selected from:
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c); or
SEQ ID NO:20 and SEQ ID NO:39 (AB-15c); or
   a combination of any of the foregoing.

The disclosure also provides, among other things, polypeptides that comprise a V_{H} comprising SEQ ID NO:4, wherein:
X₁ is not T;
X₂ is not R;
X₃ is not G;
X₄ is not N;
X₅ is not T;
X₆ is not K;
X₇ is not R;
X₈ is not L;
X₉ is not S;
X₁₀ is not I;
X₁₁ is not T;
X₁₂ is not I;
X₁₃ is not R;
X₁₄ is not R;
X₁₅ is not Y; or
X₁₆ is not V;
or any combination of the foregoing.

In some embodiments, a polypeptide comprises a V_{L} comprising SEQ ID NO:23, wherein:
X₁₇ is not L;
X₁₈ is not I;
X₁₉ is not Y;
X₂₀ is not G;
X₂₁ is not A;
X₂₂ is not Q; or
X₂₃ is not T;
or any combination of the foregoing.

In some embodiments, the disclosure provides polypeptides (e.g., polypeptides that specifically bind IL-4Rα) comprising:
a V_{H} sequence that has at least 70% sequence identity to SEQ ID NO:5;
a V_{L} sequence that has at least 70% sequence identity to SEQ ID NO:24; or
a combination thereof,
   wherein the V_{H} sequence does not comprise SEQ ID NO:5, the V_{L} sequence does not comprise SEQ ID NO:24, or both.

In some embodiments, a polypeptide disclosed herein is a fusion protein.

In some embodiments, the disclosure provides polynucleotides encoding a polypeptide disclosed herein, vectors comprising such polynucleotides, and host cells comprising such polynucleotides and/or vectors.

In some embodiments, the disclosure provides compositions comprising a polypeptide, a fusion protein or a polynucleotide disclosed herein.

In some embodiments, the disclosure provides methods of treating a subject in need thereof, comprising administering an effective amount of the composition disclosed herein.

In some embodiments, the disclosure provides methods of reducing inflammation, a symptom of an inflammatory condition, or risk of developing an inflammatory condition in a cell of a subject, comprising contacting the cell with an effective amount of the composition disclosed herein.

An example embodiment is directed to a computer-implemented method for predicting a functional property of a polypeptide. The method begins by, via a computationally binding optimized (CBO) model, for each amino acid position of an amino acid sequence of the polypeptide: (1) determining a plurality of energy scores based on the amino acid position in the amino acid sequence, (2) generating a partition function based on the plurality of energy scores determined, and (3) determining a cross-entropy metric (e.g., cross-entropy loss) based on (i) an amino acid at the amino acid position in the amino acid sequence, (ii) a maximum energy score of the plurality of energy scores determined, and (iii) the generated partition function. An analysis score of the polypeptide is generated based on each cross-entropy metric determined. The analysis score indicates a predicted functional property of the polypeptide. According to some embodiments, the polypeptide comprises an immunoglobulin heavy chain variable domain (V_{H}) comprising an amino acid sequence having at least 90% sequence identity to SEQ ID NO: 18 and an immunoglobulin light chain variable domain (V_{L}) comprising an amino acid sequence having at least 90% sequence identity to SEQ ID NO:37. In some embodiments, the polypeptide does not comprise a V_{H} comprising an amino acid sequence having 100% sequence identity to SEQ ID NO:5 and a V_{L} comprising an amino acid sequence having 100% sequence identity to SEQ ID NO:24.

In some example embodiments, the polypeptide comprises an immunoglobulin heavy chain variable domain (V_{H}) comprising an amino acid sequence having at least 70% (e.g., at least 75%, at least 80%, at least 85%, at least 90%, at least 95%, at least 96%, at least 97%, at least 98%, at least 99%, or 100%) amino acid sequence identity to SEQ ID NO:18 and an immunoglobulin light chain variable domain (V_{L}) comprising an amino acid sequence having at least about 70% amino acid sequence identity (e.g., at least 75%, at least 80%, at least 85%, at least 90%, at least 95%, at least 96%, at least 97%, at least 98%, at least 99%, or 100%) amino acid sequence identity to SEQ ID NO:37. In some example embodiments, the polypeptide comprises an immunoglobulin heavy chain variable domain (V_{H}) comprising an amino acid sequence having at least 90% amino acid sequence identity to SEQ ID NO:18 and an immunoglobulin light chain variable domain (V_{L}) comprising an amino acid sequence having at least 90% amino acid sequence identity to SEQ ID NO:37. In some example embodiments, the polypeptide comprises an immunoglobulin heavy chain variable domain (V_{H}) comprising an amino acid sequence having at least 95% amino acid sequence identity to SEQ ID NO:18 and an immunoglobulin light chain variable domain (V_{L}) comprising an amino acid sequence having at least 95% amino acid sequence identity to SEQ ID NO:37.

In an example embodiment, a given plurality of energy scores may include at least one of: a single amino acid energy score and a pairwise amino acid energy score.

According to an example embodiment, for at least one amino acid position of the amino acid sequence of the polypeptide, determining the plurality of energy scores may be further based on having substituted the amino acid at the amino acid position in the amino acid sequence with each of a plurality of different amino acids.

In an example embodiment, for at least one amino acid position of the amino acid sequence of the polypeptide, generating the partition function may be further based on a softmax function.

According to an example embodiment, at least one of: (1) predicting the functional property of the polypeptide may be implementable by a script of Appendix A or Appendix B, and (2) the CBO model may be substantially similar to a table of Appendix C.

In an example embodiment, the generated analysis score may be above a threshold. The threshold may be a score from the CBO model of one or more of a reference polypeptide that includes a V_{H} and V_{L} pair selected from:
SEQ ID NO:5 and SEQ ID NO:24 (Reference);
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c);
SEQ ID NO:20 and SEQ ID NO:39 (AB-15c); or
a combination of any of the foregoing.

According to an example embodiment, the predicted functional property may be a binding affinity for interleukin-4 receptor alpha (IL-4Rα).

In an example embodiment, the predicted functional property may be at least one of:
a binding affinity for interleukin-4 receptor alpha (IL-4Rα) characterized by a K_{D} of about 1 μM or less,
a binding affinity for IL-4Rα characterized by a kₐ of about 100×10⁵ M⁻¹ s⁻¹ or less,
a dissociation from IL-4Rα characterized by a k_{d} of about 100×10⁵ s⁻¹ or less,
a binding affinity for IL-4Rα characterized by an EC₅₀ of about 1 μM or less,
a blocking activity against IL-4 Type II signaling characterized by an IC₅₀ of about 1 μM or less,
a blocking activity against IL-13 Type II signaling characterized by an IC₅₀ of about 1 μM or less, and
an inhibitory activity against CD23 expression in B cells characterized by an IC₅₀ of about 1 μM or less.

According to an example embodiment, the predicted functional property may be at least one of:
a binding affinity for interleukin-4 receptor alpha (IL-4Rα) characterized by a K_{D} of about 0.05 to 0.5 nM or less,
a binding affinity for IL-4Rα characterized by a kₐ of about 7×10⁵ to 9×10⁵ M⁻¹ s⁻¹ or less,
a dissociation from IL-4Rα characterized by a k_{d} of about 2×10⁵ to 3×10⁻⁵ s⁻¹ or less,
a binding affinity for IL-4Rα characterized by an EC₅₀ of about 0.02 to 1.5 nM or less,
a blocking activity against IL-4 Type II signaling characterized by an IC₅₀ of about 1.1 to 7.3 nM or less,
a blocking activity against IL-13 Type II signaling characterized by an IC₅₀ of about 3 to 3.3 nM or less, and
an inhibitory activity against CD23 expression in B cells characterized by an IC₅₀ of about 8 to 50 nM or less.

In an example embodiment, the predicted functional property may be at least one of:
a binding affinity for interleukin-4 receptor alpha (IL-4Rα) characterized by a K_{D} of about 0.02 to 0.04 nM or less,
a binding affinity for IL-4Rα characterized by a kₐ of about 7.7×10⁵ to 8.9×10⁵ M⁻¹ s⁻¹ or less,
a dissociation from IL-4Rα characterized by a k_{d} of about 2.1×10⁵ to 2.6×10⁻⁵ s⁻¹ or less,
a binding affinity for IL-4Rα characterized by an EC₅₀ of about 0.04 to 1.3 nM or less,
a blocking activity against IL-4 Type II signaling characterized by an IC₅₀ of about 4.4 to 7.3 nM or less, and
an inhibitory activity against CD23 expression in B cells characterized by an IC₅₀ of about 30 to 50 nM or less.

According to an example embodiment, the predicted functional property may be at least one of:
a blocking activity against IL-4 Type II signaling characterized by an IC₅₀ of about 1.1 to 1.8 nM or less, and
an inhibitory activity against CD23 expression in B cells characterized by an IC₅₀ of about 30 to 34 nM or less.

In an example embodiment, the predicted functional property may be an inhibitory activity against CD23 expression in B cells characterized by an IC₅₀ of about 8 to 8.8 nM or less.

According to an example embodiment, the predicted functional property may relate to modulating activity of a target molecule. The target molecule may be interleukin-4 receptor alpha (IL-4Rα).

In an example embodiment, the analysis score may be a binding score.

According to an example embodiment, the predicted functional property may be a binding affinity.

In an example embodiment, the polypeptide may not comprise a heavy chain having an amino acid sequence that is identical to SEQ ID NO:104 and a light chain having an amino acid sequence that is identical to SEQ ID NO:161.

Another example embodiment is directed to a computer-based system for predicting a functional property of a polypeptide. The system includes a processor and a memory with computer code instructions stored thereon. The processor and the memory, with the computer code instructions, are configured to cause the system to implement any embodiments or combination of embodiments described herein.

Yet another embodiment is directed to a computer program product for predicting a functional property of a polypeptide. The computer program product includes a non-transitory computer-readable medium with computer code instructions stored thereon. The computer code instructions are configured, when executed by a processor, to cause an apparatus associated with the processor to implement any embodiments or combination of embodiments described herein.

It is noted that embodiments of the method, system, and computer program product may be configured to implement any embodiments or combination of embodiments described herein.

An example embodiment is directed to a polypeptide that binds interleukin-4 receptor alpha (IL-4Rα). The polypeptide is assigned a score above a threshold by an analysis via a computationally binding optimized (CBO) model. The polypeptide comprises an immunoglobulin heavy chain variable domain (V_{H}) comprising an amino acid sequence having at least 90% sequence identity to SEQ ID NO:18 and an immunoglobulin light chain variable domain (V_{L}) comprising an amino acid sequence having at least 90% sequence identity to SEQ ID NO: 37. The polypeptide does not comprise a V_{H} comprising an amino acid sequence having 100% sequence identity to SEQ ID NO:5 and a V_{L} comprising an amino acid sequence having 100% sequence identity to SEQ ID NO:24.

In an example embodiment, at least one of: (1) the analysis may be implementable by a script of Appendix A or Appendix B employing the CBO model, (2) the CBO model may be calculated using a table substantially similar to that of Appendix C, (3) the score assigned to the polypeptide may be generated using Appendix C, (4) the polypeptide may be assigned the score by the script of Appendix A or Appendix B, and (5) the score assigned to the polypeptide may be at least about-27.8.

According to an example embodiment, the polypeptide may have at least one property selected from:
a binding affinity for interleukin-4 receptor alpha (IL-4Rα) characterized by a K_{D} of about 1 μM or less,
a binding affinity for IL-4Rα characterized by a kₐ of about 100×10⁵ M⁻¹ s⁻¹ or less,
a dissociation from IL-4Rα characterized by a k_{d} of about 100×10⁻⁵ s⁻¹ or less,
a binding affinity for IL-4Rα characterized by an EC₅₀ of about 1 μM or less,
a blocking activity against IL-4 Type II signaling characterized by an IC₅₀ of about 1 μM or less,
a blocking activity against IL-13 Type II signaling characterized by an IC₅₀ of about 1 μM or less, and
an inhibitory activity against CD23 expression in B cells characterized by an IC₅₀ of about 1 μM or less.

In an example embodiment, the polypeptide may have at least one property selected from:
a binding affinity for interleukin-4 receptor alpha (IL-4Rα) characterized by a K_{D} of about 0.05 to 0.5 nM or less,
a binding affinity for IL-4Rα characterized by a kₐ of about 7×10⁵ to 9×10⁵ M⁻¹ s⁻¹ or less,
a dissociation from IL-4Rα characterized by a k_{d} of about 2×10⁵ to 3×10⁻⁵ s⁻¹ or less,
a binding affinity for IL-4Rα characterized by an EC₅₀ of about 0.02 to 1.5 nM or less,
a blocking activity against IL-4 Type II signaling characterized by an IC₅₀ of about 1.1 to 7.3 nM or less,
a blocking activity against IL-13 Type II signaling characterized by an IC₅₀ of about 3 to 3.3 nM or less, and
an inhibitory activity against CD23 expression in B cells characterized by an IC₅₀ of about 8 to 50 nM or less.

According to an example embodiment, the polypeptide may have at least one property selected from:
a binding affinity for interleukin-4 receptor alpha (IL-4Rα) characterized by a K_{D} of about 0.02 to 0.04 nM or less,
a binding affinity for IL-4Rα characterized by a kₐ of about 7.7×10⁵ to 8.9×10⁵ M⁻¹ s⁻¹ or less,
a dissociation from IL-4Rα characterized by a k_{d} of about 2.1×10⁵ to 2.6×10⁻⁵ s⁻¹ or less,
a binding affinity for IL-4Rα characterized by an EC₅₀ of about 0.04 to 1.3 nM or less,
a blocking activity against IL-4 Type II signaling characterized by an IC₅₀ of about 4.4 to 7.3 nM or less, and
an inhibitory activity against CD23 expression in B cells characterized by an IC₅₀ of about 30 to 50 nM or less.

In an example embodiment, the polypeptide has at least one property selected from:
a blocking activity against IL-4 Type II signaling characterized by an IC₅₀ of about 1.1 to 1.8 nM or less, and
an inhibitory activity against CD23 expression in B cells characterized by an IC₅₀ of about 30 to 34 nM or less.

According to an example embodiment, the polypeptide may have an inhibitory activity against CD23 expression in B cells characterized by an IC₅₀ of about 8 to 8.8 nM or less.

In an example embodiment, the threshold may be a score from the CBO model of one or more of a reference polypeptide that includes a V_{H} and V_{L} pair selected from:
SEQ ID NO:5 and SEQ ID NO:24 (Reference);
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c);
SEQ ID NO:20 and SEQ ID NO:39 (AB-15c); or
a combination of any of the foregoing.

According to an example embodiment, the polypeptide may not comprise a heavy chain having an amino acid sequence that is identical to SEQ ID NO:104 and a light chain having an amino acid sequence that is identical to SEQ ID NO:161.

Another example embodiment is directed to a polypeptide that binds human interleukin-4 receptor alpha (IL-4Rα). The polypeptide is selected by a method comprising:
evaluating a plurality of candidate polypeptides using a computationally binding optimized (CBO) model by, for each candidate polypeptide of the plurality of candidate polypeptides:
   for each amino acid position of an amino acid sequence of the candidate polypeptide:
      determining a plurality of energy scores based on the amino acid position in the amino acid sequence;
      generating a partition function based on the plurality of energy scores determined; and
      determining a cross-entropy metric based on (i) an amino acid at the amino acid position in the amino acid sequence, (ii) a maximum energy score of the plurality of energy scores determined, and (iii) the generated partition function; and
   generating an analysis score of the candidate polypeptide based on each cross-entropy metric determined, the analysis score indicating a functional property of the polypeptide's ability to bind to human IL-4Rα; and
selecting a given candidate polypeptide from among the plurality of candidate polypeptides based a result of the evaluating,
wherein the polypeptide comprises an immunoglobulin heavy chain variable domain (V_{H}) comprising an amino acid sequence having at least 90% sequence identity to SEQ ID NO:18 and an immunoglobulin light chain variable domain (V_{L}) comprising an amino acid sequence having at least 90% sequence identity to SEQ ID NO:37, and
wherein the polypeptide does not comprise a V_{H} comprising an amino acid sequence having 100% sequence identity to SEQ ID NO:5 and a V_{L} comprising an amino acid sequence having 100% sequence identity to SEQ ID NO:24.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

In the drawings, “Reference” refers to the Reference Antibody.

FIG. 1 depicts the amino acid sequence of an epitope within the N-terminal receptor alpha domain of a human interleukin-4 receptor alpha (IL-4Rα) (an example sequence of IL-4Rα is set forth in SEQ ID NO:1). The epitope residues bound by the Reference Antibody disclosed herein are indicated by asterisks (*). Also see non-limiting examples of IL-4Rα sequences (SEQ ID NOs: 1-3) in Table 1.

FIGS. 2A and 2B depict an alignment of non-limiting examples of heavy chain variable domain (V_{H}) amino acid sequences that are useful in polypeptides as disclosed herein. The heavy chain complementarity determining region (HCDR) amino acid sequences as determined by ImMunoGeneTics (IMGT) numbering (www.imgt.org/IMGTScientificChart/Nomenclature/IMGT-FRCDRdefinition.html, also accessible at www.imgt.org/) are underlined. The bold letters indicate non-limiting examples of variable residues (designated throughout this disclosure by “Xₙ”) in the depicted sequences. An asterisk (*) indicates paratope residues. Paratope residues were defined as antibody residues in the Reference that, when bound to IL-4Rα, are within 5 angstroms (Å) of the antigen. Also see V_{H} consensus sequence (SEQ ID NO:4) for the Reference Antibody and AB-1 to AB-15 in Table 2. The antibody sequences were computationally generated using information from the sequence and structure of a reference polypeptide (“Reference”).

FIGS. 3A and 3B depict an alignment of non-limiting examples of light chain variable domain (V_{L}) amino acid sequences that are useful in polypeptides as disclosed herein. The light chain complementarity determining region (LCDR) amino acid sequences as determined by IMGT numbering are underlined. The bold letters indicate non-limiting examples of variable residues (designated throughout this disclosure by “Xₙ”) in the depicted sequences. An asterisk (*) indicates paratope residues. Paratope residues were defined as antibody residues in the Reference that, when bound to IL-4Rα, are within 5 angstroms (Å) of the antigen. Also see V_{L} consensus sequence (SEQ ID NO:23) for the Reference Antibody and AB-1 to AB-15 in Table 2. The antibody sequences were computationally generated using information from the sequence and structure of a reference polypeptide (“Reference”).

FIGS. 4A-4C show antibody (mAb) binding in indirect ELISA binding assays. FIG. 4A shows hIgG4 (“c” denotes wild-type IgG4) antibody binding as absorbance at 450 nm (A450) at varying antibody concentrations (nM). The Reference antibody is an IgG4 wild-type antibody. FIG. 4B shows hIgG1 antibody binding as absorbance at 450 nm (A450) at varying antibody concentrations (nM). FIG. 4C shows hIgG4 (WT, YTE, or LS variant; “a” denotes IgG4 YTE variant, “b” denotes IgG4 LS variant, “c” denotes wild-type IgG4) antibody binding as absorbance at 450 nm (A450) at varying antibody concentrations (nM). The negative control was Isotype IgG4.

FIGS. 5A-5C show results from an assessment of the ability of antibodies to block IL-4-mediated activation of IL-4Rα via a HEK (human embryonic kidney)-Blue colorimetric assay (HEK-Blue (STAT6) assay). FIG. 5A shows hIgG4 (WT, YTE, or LS variant; “a” denotes IgG4 YTE variant, “b” denotes IgG4 LS variant, “c” denotes wild-type IgG4) antibody blocking as percent inhibition (% inhibition) at varying antibody (mAb) concentrations (nM). FIG. 5B shows hIgG1 (“d” denotes IgG1) antibody blocking as percent inhibition at varying mAb concentrations (nM). FIG. 5C shows hIgG4 (WT, YTE, or LS variant) antibody blocking as percent inhibition at varying antibody mAb concentrations (nM). Isotype IgG4 was the negative control.

FIGS. 6A-6D show results from an IL-4Rα Human Primary Cell-based Assay (CD23 Inhibition assay) that used isolated primary B cells from four different donors. Percent inhibition was calculated from mean fluorescence intensity (MFI).

FIG. 7 shows results from a Ramos CD23 inhibition assay. Ramos cells were treated with an antibody or isotype control, then stained to identify CD23 inhibition by anti-IL-4Rα antibodies. Percent inhibition was calculated from mean fluorescence intensity (MFI).

FIGS. 8A-8D depict results from blocking of IL-4 stimulation of Signal Transducer and Activator of Transcription 6 phosphorylation (pSTAT6) in whole human blood. The Reference and AB-13c are IgG4 wild-type antibodies. FIGS. 8A and 8C-8D show that with IL-4 stimulation (1 ng/mL), there is a dose-dependent decrease in pSTAT6 with both Reference Antibody (hIgG4) and AB-13c (hIgG4) in whole blood from three healthy donors in both B cells (CD19+) and T cells (CD4+) (Donor 1, FIG. 8A; Donor 2, FIG. 8C; Donor 3, FIG. 8D). FIG. 8B shows representative images of a dose-dependent decrease in pSTAT6 expression in B and T cells in response to treatment with AB-13c or the Reference Antibody.

FIGS. 9A-9D depict evaluation of IL-4Rα target engagement in primary cells via functional inhibition of relevant atopic dermatitis (AD) biomarker Thymus- and Activation-Regulated Chemokine (TARC or CCL17). The Reference and AB-13c are IgG4 wild-type antibodies. FIG. 9A shows dose-dependent TARC stimulation seen with IL-4 in peripheral blood mononuclear cells (PBMCs) starting at 0.1 ng/ml of IL-4 with peak induction occurring at doses ≥1 ng/ml. FIGS. 9B-9D show that AB-13c (hIgG4 WT) results in a dose-dependent decrease of TARC release that is comparable to the Reference when cells are stimulated with 1 ng/ml (FIG. 9B), 10 ng/mL (FIG. 9C), or 100 ng/ml (FIG. 9D) of IL-4.

FIG. 10 is a flowchart of a method for predicting a functional property of a polypeptide according to an example embodiment.

FIG. 11 is a schematic view of a computer network in which embodiments may be implemented.

FIG. 12 is a block diagram illustrating an example embodiment of a computer node in the computer network of FIG. 11.

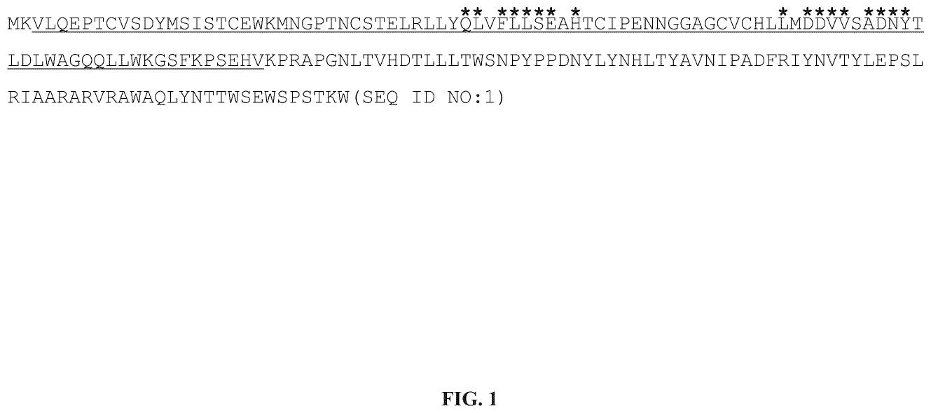

### DETAILED DESCRIPTION

A description of example embodiments follows.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the disclosure can be practiced without one or more of the specific details or practiced with other methods, protocols, reagents, cell lines and animals. The disclosure is not limited by the illustrated ordering of acts or events, as some acts may occur in different orders and/or concurrently with other acts or events. Furthermore, not all illustrated acts, steps or events are required to implement a methodology in accordance with the disclosure.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting.

When introducing elements disclosed herein, the articles “a,” “an,” “the,” and “said” are intended to mean that there are one or more of the elements. Further, the one or more elements may be the same or different. For example, unless the context clearly indicates otherwise, “a polypeptide” includes a single polypeptide, and two or more polypeptides.

Throughout this specification and the claims which follow, unless the context requires otherwise, the term “comprise,” and variations such as “comprises” and “comprising”, will be understood to imply the inclusion of, e.g., a stated integer or step or group of integers or steps, but not the exclusion of any other integer or step or group of integer or step. When used herein, the term “comprising” can be substituted with the term “containing” or “including.”

As used herein, the term “consisting of” excludes any element, step, or ingredient not specified in the claim element. When used herein, the term “consisting essentially of” does not exclude materials or steps that do not materially affect the basic and novel characteristics of the claim.

Any of the terms “comprising,” “containing,” “including,” and “having,” whenever used herein in the context of an aspect or embodiment disclosed herein, can in some embodiments, be replaced with the term “consisting of,” or “consisting essentially of” to vary scopes disclosed herein.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

It should be understood that for all numerical bounds describing some parameter in this application, such as “about,” “at least,” “less than,” “fewer than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, et cetera.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used herein, the term “about” means within an acceptable error range for a particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. Alternatively, “about” can mean a range of ±20%, e.g., ±10%, ±5% or ±1% of a given value. It is to be understood that the term “about” can precede any particular value specified herein, except for particular values used in the Exemplification. When “about” precedes a range, as in “90-99.9%,” the term “about” should be read as applying to both given values of the range, such that “about 90-99.9%” means about 90% to about 99.9%.

As used herein, the term “polypeptide” refers to a polymer of at least two amino acids covalently linked by an amide bond, regardless of length or post-translational modification (e.g., glycosylation or phosphorylation). A polypeptide can comprise any suitable L- and/or D-amino acid, for example, common α-amino acids (e.g., alanine, glycine, valine), non-α-amino acids (e.g., β-alanine, 4-aminobutyric acid, 6-aminocaproic acid, sarcosine, statine), and unusual amino acids (e.g., citrulline, homocitruline, homoserine, norleucine, norvaline, ornithine). The amino, carboxyl, and/or other functional groups on a polypeptide can be free (e.g., unmodified) or protected with a suitable protecting group. Suitable protecting groups for amino and carboxyl groups, and methods for adding or removing protecting groups are known in the art and are disclosed in, for example, Green and Wuts, “Protecting Groups in Organic Synthesis,” John Wiley and Sons, 1991. The functional groups of a polypeptide can also be derivatized (e.g., alkylated) or labeled (e.g., with a detectable label, such as a fluorogen or a hapten) using methods known in the art. A polypeptide can comprise one or more modifications (e.g., amino acid linkers, acylation, acetylation, amidation, methylation, terminal modifiers (e.g., cyclizing modifications), N-methyl-«-amino group substitution), if desired. In addition, a polypeptide can be an analog of a known and/or naturally-occurring peptide, for example, a peptide analog having conservative amino acid residue substitution(s). The terms “polypeptide” and “protein” are used interchangeably herein.

As used herein, a “polynucleotide” is defined as a plurality of nucleotides and/or nucleotide analogs linked together in a single molecule. In some embodiments, a polynucleotide disclosed herein comprises deoxyribonucleotides. In some embodiments, the polynucleotide comprises ribonucleotides. Non-limiting examples of polynucleotides include single-, double- or multi-stranded DNA or RNA, DNA-RNA hybrids (e.g., each “T” position may be independently substituted by a “U” or vice versa), or a polymer comprising purine and pyrimidine bases, or other natural, chemically, or biochemically modified, non-natural, or derivatized nucleotide bases. The backbone of the polynucleotide can comprise sugars and phosphate groups, modified or substituted sugar or phosphate groups, a polymer of synthetic subunits such as phosphoramidates, or a combination thereof.

As used herein, the term “sequence identity” refers to the extent to which two nucleotide sequences have the same residues at the same positions when the sequences are aligned to achieve a maximal level of identity, expressed as a percentage. For sequence alignment and comparison, typically one sequence is designated as a reference sequence, to which test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 70% sequence identity when, upon alignment to achieve a maximal level of identity, the test sequence has the same nucleotide residue at 70% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples include the local homology algorithm of Smith & Waterman, Adv. Appl. Math. 2:482 (1981), the homology alignment algorithm of Needleman & Wunsch, J. Mol. Biol. 48:443 (1970), the search for similarity method of Pearson & Lipman, Proc. Nat'l. Acad. Sci. USA 85:2444 (1988), computerized implementations of these algorithms (GAP, BESTFIT, FASTA, and TFASTA in the Wisconsin Genetics Software Package, Genetics Computer Group, 575 Science Dr., Madison, Wis.), and visual inspection (see generally Ausubel et al., Current Protocols in Molecular Biology).

When using a sequence comparison algorithm, test and reference sequences are input into a computer, subsequent coordinates are designated, if necessary, and sequence algorithm program parameters are designated. The sequence comparison algorithm then calculates the percent sequence identity for the test sequence(s) relative to the reference sequence, based on the designated program parameters. A commonly used tool for determining percent sequence identity is Protein Basic Local Alignment Search Tool (BLASTP) available through National Center for Biotechnology Information, National Library of Medicine, of the United States National Institutes of Health (Altschul et al., 1990).

As used herein, the term “substantially similar to” refers to a polypeptide disclosed herein that is substantially similar in amino acid sequence (e.g., has at least about 80%, at least about 90%, at least about 95%, at least about 96%, at least about 97%, at least about 98%, or at least about 99% of the amino acid residues amino acid sequence identity) and substantially preserves one or more functional properties of a specified polypeptide disclosed herein. In some embodiments, the one or more functional properties are selected from, without limitation, a substantially similar binding affinity, a substantially similar binding specificity, a substantially similar inhibitory activity, a substantially similar neutralization activity, and a substantially similar self-association property.

As used herein, a “complementarity determining region (CDR)” encompasses any CDR defined by an art-recognized method for identifying the CDR residues on an antibody. See, e.g., Kabat, E. A., et al., (1991) Sequences of Proteins of Immunological Interest, Fifth Edition, U.S. Department of Health and Human Services, NIH Publication No. 91-3242, Chothia et al., (1989) Nature 342:877; Chothia, C. et al., (1987) J. Mol. Biol. 196:901-917; Al-lazikani et al., (1997) J. Molec. Biol. 273:927-948; and Almagro, J. Mol. Recognit. 17:132-143 (2004). See also hgmp.mrc.ac.uk and bioinf.org.uk/abs. Two antibodies are determined to have the same CDR as one another with respect to a HCDR1, HCDR2, HCDR3, LCDR1, LCDR2 and/or LCDR3, when the identity of that CDR is determined for both antibodies using the same method.

The extent of the framework region and the CDRs of an antibody can be identified using one of several suitable methodologies that are well known in the art, for example, by the Kabat definition, the Chothia definition, the AbM definition, and/or the contact definition. Publicly and/or commercially available tools for identifying framework and/or CDR regions include, IgBlast (accessible at www.ncbi.nlm.nih.gov/igblast/), Scaligner (available from drugdesigntech at www.scaligner.com/), IMGT rules and/or tools (see, for example, www.imgt.org/IMGTScientificChart/Nomenclature/IMGT-FRCDRdefinition.html, also accessible at www.imgt.org/), Chothia Canonical Assignment (accessible at www.bioinf.org.uk/abs/chothia.html), Antigen receptor Numbering And Receptor CalssificatiIon (ANARCI, accessible at opig.stats.ox.ac.uk/webapps/newsabdab/sabpred/anarci/), or see Vered Kunik, et al, Nucleic Acids Research, Volume 40, Issue W1, 1 Jul. 2012, Pages W521-W524).

As used herein, the term “antibody mimetic” refers to polypeptides capable of mimicking an antibody's ability to bind an antigen, but structurally differ from native antibody structures. Examples of antibody mimetics include, but not limited to, Adnectins, Affibodies, Affilins, Affimers, Affitins, Alphabodies, Anticalins, Avimers, DARPins, Fynomers, Kunitz domain peptides, monobodies, nanobodies, nanoCLAMPs, and Versabodies.

The term “subject” refers to a mammalian subject, preferably human, diagnosed with or suspected of having an inflammatory condition in which the interleukin 4 (IL-4)/interleukin 13 (IL-13) axis has been implicated, such as, but not limited to: asthma, eczema (atopic dermatitis), food allergy, prurigo nodularis, chronic rhinosinusitis with nasal polyps, keloids, eosinophilic esophagitis, prostate cancer, chronic urticaria, bullous pemphigoid, localized scleroderma, alopecia areata, ulcerative colitis, aspirin-exacerbated respiratory disease, metastatic non-small cell lung cancer, and Netherton syndrome.

The phrase “pharmaceutically acceptable” means that the substance or composition the phrase modifies is, within the scope of sound medical judgment, suitable for use in contact with the tissues of humans and lower animals without undue toxicity, irritation, allergic response, and the like, and are commensurate with a reasonable benefit/risk ratio.

As used herein, the term “pharmaceutically acceptable salt” refers to those salts which are, within the scope of sound medical judgment, suitable for use in contact with the tissues of mammals without undue toxicity, irritation, allergic response, and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, the relevant teachings of which are incorporated herein by reference in their entirety. Pharmaceutically acceptable salts of the agents/compounds described herein include salts derived from suitable inorganic and organic acids, and suitable inorganic and organic bases.

Examples of salts derived from suitable acids include salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable salts derived from suitable acids include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N⁺((C₁-C₄)alkyl)₄ salts. Representative alkali or alkaline earth metal salts include sodium, lithium, potassium, calcium, magnesium, barium, and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

“Pharmaceutically acceptable carrier” refers to a non-toxic carrier or excipient that does not destroy the pharmacological activity of the agent with which it is formulated and is nontoxic when administered in doses sufficient to deliver a therapeutic amount of the agent. Pharmaceutically acceptable carriers that may be used in the compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

“Treating” or “treatment,” as used herein, refers to taking steps to deliver a therapy to a subject, such as a mammal, in need thereof (e.g., as by administering to a mammal one or more therapeutic agents). “Treating” or “treatment” includes inhibiting the disease or condition (e.g., as by slowing or stopping its progression or causing regression of the disease or condition) and relieving the symptoms resulting from the disease or condition. The term “treating,” or “treatment” refers to the medical management of a subject with the intent to improve, ameliorate, stabilize (i.e., not worsen), prevent, or cure a disease, pathological condition, or disorder-such as the particular indications exemplified herein. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. In some embodiments, a pharmaceutical composition suitable for use in methods disclosed herein further comprises one or more pharmaceutically acceptable carriers.

“Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the agent (e.g., polypeptide) is administered. Such vehicles may be liquids, such as water and oils, including those of petroleum, animal, vegetable or synthetic origin, such as peanut oil, soybean oil, mineral oil, sesame oil, and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating, and coloring agents, etc. The concentration of the agent in such pharmaceutical formulation may vary widely, i.e., from less than about 0.5%, to at least about 1%, or to as much as 15% or 20%, 25%, 30%, 35%, 40%, 45% or 50% by weight. The concentration will be selected primarily based on required dose, fluid volumes, viscosities, etc., according to the mode of administration. Suitable vehicles and formulations, inclusive of other human proteins, e.g., human serum albumin, are described, for example, in Remington: The Science and Practice of Pharmacy, 21^{st} Edition, Troy, D. B. ed., Lipincott Williams and Wilkins, Philadelphia, PA 2006, Part 5, Pharmaceutical Manufacturing: 691-1092 (e.g., pages 958-89).

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers are acetic acid, citric acid, formic acid, succinic acid, phosphoric acid, carbonic acid, malic acid, aspartic acid, histidine, boric acid, Tris buffers, HEPPSO, and HEPES.

Non-limiting examples of antioxidants are ascorbic acid, methionine, cysteine hydrochloride, sodium bisulfate, sodium metabisulfite, sodium sulfite, lecithin, citric acid, ethylenediamine tetraacetic acid (EDTA), sorbitol, and tartaric acid.

Non-limiting examples of amino acids are histidine, isoleucine, methionine, glycine, arginine, lysine, L-leucine, tri-leucine, alanine, glutamic acid, L-threonine, and 2-phenylamine.

Non-limiting examples of surfactants are polysorbates (e.g., polysorbate-20 or polysorbate-80); polyoxamers (e.g., poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g., PLURONICS™, PF68, etc.).

Non-limiting examples of preservatives are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl, and the like), benzalkonium chloride, benzethonium chloride, sodium dehydroacetate, and thimerosal, or mixtures thereof.

Non-limiting examples of saccharides are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, or iso-maltulose.

Non-limiting examples of salts are acid addition salts and base addition salts. Acid addition salts include those derived from nontoxic inorganic acids, such as hydrochloric, nitric, phosphoric, sulfuric, hydrobromic, hydroiodic, phosphorous, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, and the like. Base addition salts include those derived from alkaline earth metals, such as sodium, potassium, magnesium, calcium, and the like, as well as from nontoxic organic amines, such as N,N′-dibenzylethylenediamine, N-methylglucamine, chloroprocaine, choline, diethanolamine, ethylenediamine, procaine, and the like. In some embodiments, the salt is sodium chloride (NaCl).

Agents (e.g., polypeptides) described herein may be prepared in accordance with standard procedures and are administered at dosages that are selected to reduce, prevent, or eliminate, or to slow or halt progression of, a condition being treated (see, e.g., Remington's Pharmaceutical Sciences, Mack Publishing Company, Easton, PA, and Goodman and Gilman's The Pharmaceutical Basis of Therapeutics, McGraw-Hill, New York, N. Y., the contents of which are incorporated herein by reference, for a general description of methods for administering various agents for human therapy).

“Administering” or “administration,” as used herein, refers to providing a compound, composition, or pharmaceutically acceptable salt thereof described herein to a subject in need of treatment or prevention. Administering can be performed, for example, once, a plurality of times, and/or over one or more extended periods. Administration includes both direct administration (including self-administration), and indirect administration (including an act of prescribing a drug or directing a subject to consume an agent). For example, as used herein, one (e.g., a physician) who instructs a subject (e.g., a human patient) to self-administer an agent (e.g., a drug), or to have an agent administered by another and/or who provides a patient with a prescription for a drug is administering an agent to a subject.

“A therapeutically effective amount” or “an effective amount” refers to an amount effective, at dosages and for periods of time necessary, to achieve a desired therapeutic or biological result (e.g., treatment, healing, inhibition or amelioration of physiological response or condition, etc.). A therapeutically effective amount may vary according to factors such as the disease state, age, sex, and weight of the individual, and the ability of a therapeutic or a combination of therapeutics to elicit a desired response in the individual.

### Polypeptides

### Interleukin-4 Receptor Subunit Alpha (IL-4Rα)

As used herein, the terms “IL-4Rα” and “IL-4R” are used interchangeably. As used herein, IL-4Rα includes wild-type IL-4Rα proteins (e.g., wild-type human IL-4Rα proteins or homologs thereof) and subunits, domains (e.g., N-terminal cytokine homology domain (CHR) and ectodomain of Ra chain), and truncated forms thereof, mutant and engineered versions of full-length and truncated IL-4Rα proteins, and modified forms (e.g., post-translationally modified forms) of full-length and truncated IL-4Rα proteins. Non-limiting examples of IL-4Rα sequences include SEQ ID NOs: 1-3 (Table 1).

In some embodiments, a polypeptide binds to a wild-type IL-4Rα protein.

In some embodiments, a polypeptide binds to a mutant or engineered IL-4Rα protein. In some embodiments, a mutant or engineered IL-4Rα protein comprises an amino acid sequence having at least about 90% sequence identity to a wildtype IL-4Rα protein, for example, having at least about: 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% sequence identity to a wildtype IL-4Rα protein. In some embodiments, a mutant or engineered IL-4Rα protein comprises an amino acid sequence having about 90-99.9%, 90-99.8%, 92-99.8%, 92-99.6%, 94-99.6%, 94-99.5%, 95-99.5%, 95-99.4%, 96-99.4%, 96-99.2%, 97-99.2%, or 97-99% sequence identity to a wildtype IL-4Rα protein.

In some embodiments, a polypeptide binds to an IL-4Rα protein that comprises a mutation associated with a disease (e.g., severe asthma).

In some embodiments, a polypeptide binds to a modified IL-4Rα protein.

In some embodiments, a polypeptide is capable of binding to one or more epitope residues in an IL-4Rα protein, for example, 1, 2, 3, 4, 5, 6, 7, 8, 9, 10, 11, 12, 13, 14, 15, 16, or 17 epitope residues of an IL-4Rα protein. In some embodiments, a polypeptide is capable of binding to one or more epitope residues (e.g., 1, 2, 3, 4, 5, 6, 7, 8, 9, 10, 11, 12, 13, 14, 15, 16, or all 17 residues) selected from Q38, L39, F41, L42, L43, S44, E45, H47, L64, D66, D67, V68, V69, A71, D72, N73, and Y74 of SEQ ID NO:1.

In some embodiments, a polypeptide binds to one or more epitope residues (e.g., 1, 2, 3, 4, 5, 6, 7, 8, 9, 10, 11, 12, 13, 14, 15, 16, or all 17 residues) selected from Q38, L39, F41, L42, L43, S44, E45, H47, L64, D66, D67, V68, V69, A71, D72, N73, and Y74 of SEQ ID NO:1.

### Comparator Polypeptides

As used herein, the term “comparator” or “comparator polypeptide” refers to a polypeptide (e.g., immunoglobulin molecule) that specifically binds to an IL-4Rα protein and is not a polypeptide disclosed herein. The sequence of a comparator polypeptide and a polypeptide disclosed herein may be compared to illustrate structural differences between them (e.g., differences at one or more amino acid positions, such as amino acid substitutions). Polypeptides disclosed herein have more than insubstantial differences (e.g., one or more substantial differences) in comparison to a comparator polypeptide, such that, polypeptides disclosed herein will, under controlled conditions, exhibit one or more (i.e., one, two, or all three) of: a different function, in a different way, to achieve a different result, in comparison to a comparator polypeptide. A comparator polypeptide may vary from a polypeptide disclosed herein by one or more amino acids, e.g., in some embodiments, by 1, 2, 3, 4, 5, 6, 7, 8, 9, 10, 11, 12, 13, 14, 15, 16, 17, 18, 19, 20, or more amino acids. In some embodiments, a comparator polypeptide diverges from a polypeptide disclosed herein by at least about: 0.4%, 0.8%, 1%, 2%, 3%, 4%, 5%, 6%, 7%, 8%, 9%, 10%, 15%, 20%, 25%, 30%, 35%, 40%, 45%, 50%, 55%, or more amino acid percent identity.

In some embodiments, a comparator polypeptide comprises:
a) a heavy chain complementarity determining region 1 (HCDR1), a heavy chain complementarity determining region 2 (HCDR2) and a heavy chain complementarity determining region 3 (HCDR3) sequences of SEQ ID NO:43, SEQ ID NO:47 and SEQ ID NO:55, respectively;
b) a light chain complementarity determining region 1 (LCDR1), a light chain complementarity determining region 2 (LCDR2) and a light chain complementarity determining region 3 (LCDR3) sequences of SEQ ID NO:72, SEQ ID NO:84 and SEQ ID NO:87, respectively; or both a) and b).

In some embodiments, a comparator polypeptide comprises:
a) a HCDR1, a HCDR2 and a HCDR3 sequences of SEQ ID NO:43, SEQ ID NO:47 and SEQ ID NO:55, respectively; and
b) a LCDR1, a LCDR2 and a LCDR3 sequences of SEQ ID NO:72, SEQ ID NO:84 and SEQ ID NO:87, respectively.

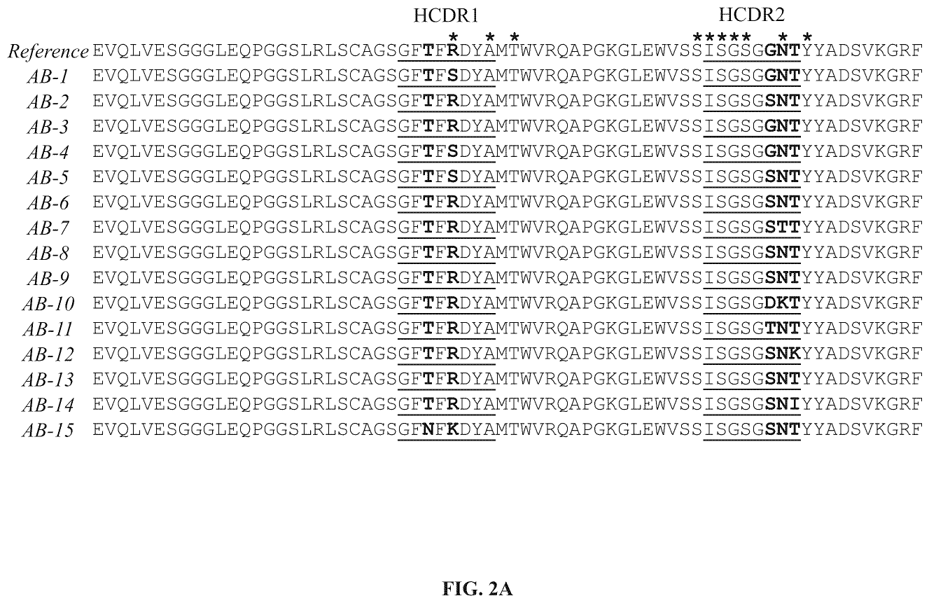

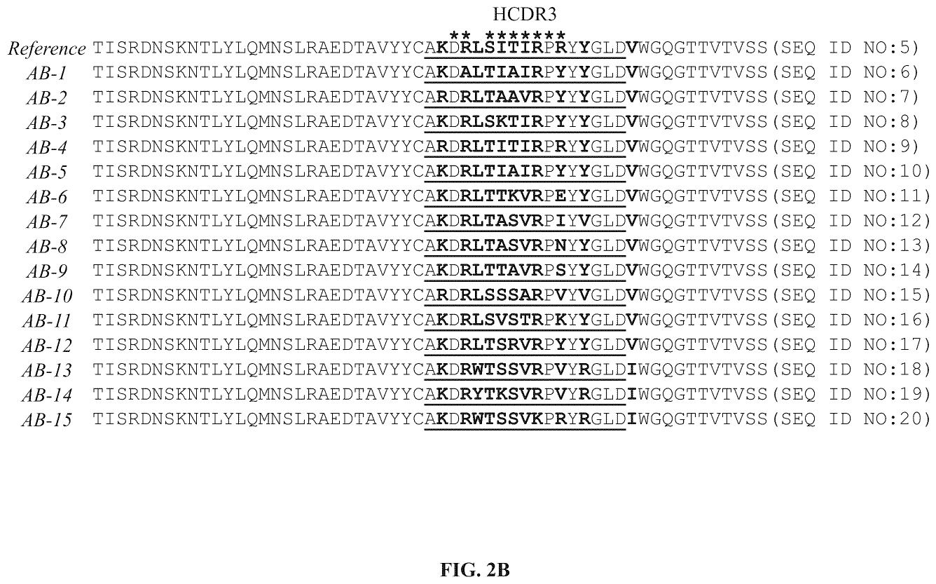

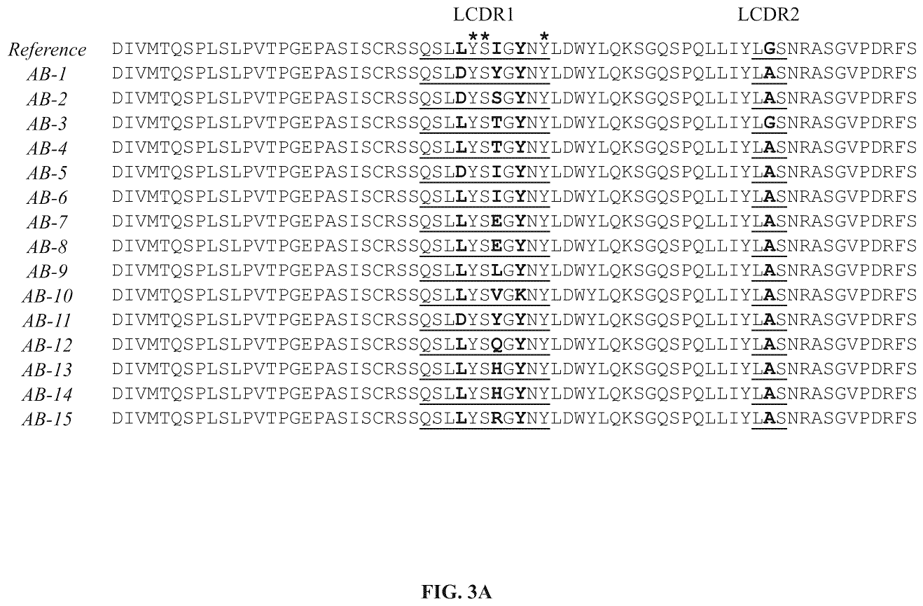

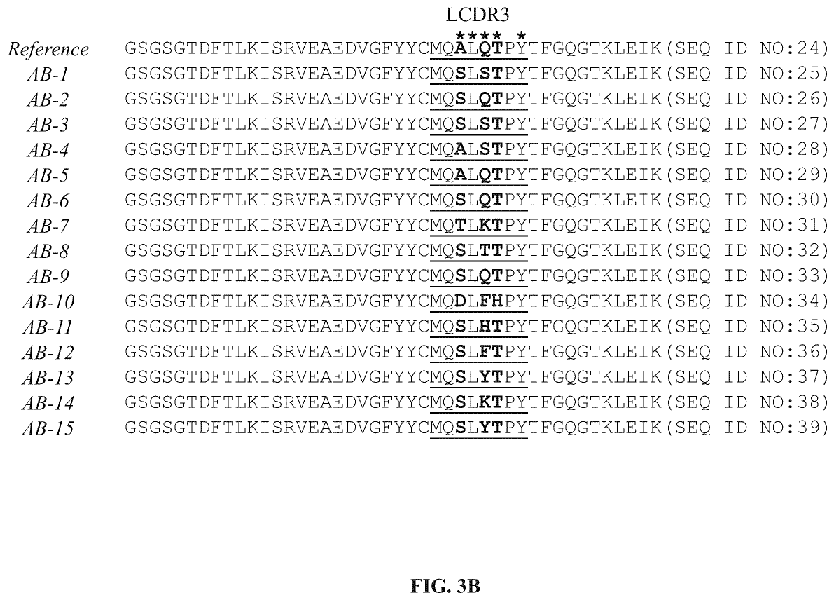

See Table 3 and FIGS. 2A-2B for SEQ ID NOs: 43, 47 and 55. See Table 3 and FIGS. 3A-3B for SEQ ID NOs: 72, 84 and 87.

In some embodiments, a comparator polypeptide comprises:
a) an immunoglobulin heavy chain variable region (V_{H}) domain comprising the amino acid sequence of SEQ ID NO:5;
b) an immunoglobulin light chain variable region (V_{L}) domain comprising the amino acid sequence of SEQ ID NO:24; or both a) and b).

In some embodiments, a comparator polypeptide comprises:
a) a V_{H} domain comprising the amino acid sequence of SEQ ID NO:5; and
b) a V_{L} domain comprising the amino acid sequence of SEQ ID NO:24.

See Table 2 and FIGS. 2A-2B for SEQ ID NO:5. See Table 2 and FIGS. 3A-3B for SEQ ID NO:24.

In some embodiments, a comparator polypeptide comprises:
a) a heavy chain comprising the amino acid sequence of SEQ ID NO:104;
b) a light chain comprising the amino acid sequence of SEQ ID NO:161; or both a) and b).

In some embodiments, a comparator polypeptide is an antibody, referred to herein as the “Reference Antibody” or “Reference.” The Reference Antibody comprises:
a) a heavy chain comprising the amino acid sequence of SEQ ID NO:104; and
b) a light chain comprising the amino acid sequence of SEQ ID NO:161.

See Table 5 for SEQ ID NO:104. See Table 6 for SEQ ID NO:161.

The Reference Antibody is an antibody that binds IL-4Rα protein and blocks IL-4 and IL-13 activity.

### Variable Domains

In some embodiments, a polypeptide comprises an immunoglobulin heavy chain variable region (V_{H}), an immunoglobulin light chain variable region (V_{L}), or a V_{H} and a V_{L}.

In some embodiments, a polypeptide comprises a V_{H}. In some embodiments, a polypeptide comprises a V_{H} that is humanized, contains human framework regions, or both.

In some embodiments, a polypeptide comprises a V_{H} having less than 100% sequence identity to the amino acid sequence of SEQ ID NO:5.

In some embodiments, a polypeptide comprises a V_{H} that has at least about 70% sequence identity to the amino acid sequence of SEQ ID NO:5. In some embodiments, a polypeptide comprises a V_{H} that has at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO:5. In some embodiments, a polypeptide comprises a V_{H} that has at least about 85% sequence identity to the amino acid sequence of SEQ ID NO:5. In some embodiments, a polypeptide comprises a V_{H} that has at least about 90% sequence identity to the amino acid sequence of SEQ ID NO:5.

In some embodiments, a polypeptide comprises a V_{H} that comprises at least one amino acid substitution (e.g., at least one conservative substitution such as highly conservative substitution) relative to the amino acid sequence of SEQ ID NO:5. For example, the number of amino acid substitutions can be at least about: 2, 3, 4, 5, 6, 7, 8, 9, 10, 11, 12, 13, 14, 15, 16, 17, 18, 19, or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11, or 9-11. In some embodiments, a polypeptide comprises a V_{H} that comprises about 1-10 amino acid substitutions, relative to the amino acid sequence of SEQ ID NO: 5. In some embodiments, the at least one amino acid substitution replaces only a heavy chain complementarity determining region 1 (HCDR1), a heavy chain complementarity determining region 2 (HCDR2), and/or a heavy chain complementarity determining region 3 (HCDR3) residue, of SEQ ID NO:5. In some embodiments, the at least one amino acid substitution replaces only a non-CDR residue (e.g., within a framework region), of SEQ ID NO:5.

In some embodiments, an amino acid substitution is a conservative substitution. The term “a conservative amino acid substitution” or “a conservative substitution” refers to an amino acid substitution having a value of 0 or greater in BLOSUM62.

In some embodiments, an amino acid substitution is a highly conservative substitution. The term “a highly conservative amino acid substitution” or “a highly conservative substitution” refers to an amino acid substitution having a value of at least 1 (e.g., at least 2) in BLOSUM62.

In some embodiments, a polypeptide comprises a V_{H} that has 100% sequence identity to the amino acid sequence of SEQ ID NO:5. In some embodiments, a polypeptide comprises a V_{H} that comprises the amino acid sequence of SEQ ID NO:5.

In some embodiments, a polypeptide comprises a Vu that has at least about 70% sequence identity to the amino acid sequence of any one or more of SEQ ID NOs: 6-20. The sequences identified as SEQ ID NOs: 6-20 are shown in Table 2, which correspond to human V_{H} domains. In some embodiments, a polypeptide comprises a V_{H} that has at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of any one or more of SEQ ID NOs: 6-20. In some embodiments, a V_{H} has at least about 85% sequence identity to the amino acid sequence of any one or more of SEQ ID NOs: 6-20. In some embodiments, a polypeptide comprises a V_{H} that has at least about 90% sequence identity to the amino acid sequence of any one or more of SEQ ID NOs: 6-20.

In some embodiments, a polypeptide comprises a V_{H} that comprises at least one amino acid substitution relative to the amino acid sequence of any one or more of SEQ ID NOs: 6-20. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11, or 9-11. In some embodiments, a polypeptide comprises a V_{H} that comprises about 1-10 amino acid substitutions, relative to the amino acid sequence of any one or more of SEQ ID NOs: 6-20.

In some embodiments, a polypeptide comprises a V_{H} that has 100% sequence identity to the amino acid sequence of any one of SEQ ID NO:6-20. In some embodiments, a polypeptide comprises a V_{H} that comprises the amino acid sequence of any one of SEQ ID NO:6-20.

In some embodiments, a polypeptide comprises a V_{H} that has at least about 70% sequence identity to the amino acid sequence of SEQ ID NO:18. In some embodiments, a polypeptide comprises a V_{H} that has at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO:18. In some embodiments, a V_{H} has at least about 85% sequence identity to the amino acid sequence of SEQ ID NO:18. In some embodiments, a polypeptide comprises a V_{H} that has at least about 90% sequence identity to the amino acid sequence of SEQ ID NO:18.

In some embodiments, a polypeptide comprises a V_{H} that comprises at least one amino acid substitution relative to the amino acid sequence of SEQ ID NO:18. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11, or 9-11. In some embodiments, a polypeptide comprises a V_{H} that comprises about 1-10 amino acid substitutions, relative to the amino acid sequence of SEQ ID NO:18.

In some embodiments, a polypeptide comprises a V_{H} that has 100% sequence identity to the amino acid sequence of SEQ ID NO:18. In some embodiments, a polypeptide comprises a V_{H} that comprises the amino acid sequence of SEQ ID NO:18.

In some embodiments, a polypeptide comprises a V_{L}. In some embodiments, a polypeptide comprises a V_{L} that is humanized, contains human framework regions, or both.

In some embodiments, a polypeptide comprises a V_{L} having less than 100% sequence identity to the amino acid sequence of SEQ ID NO:24.

In some embodiments, a polypeptide comprises a V_{L} that has at least about 70% sequence identity to the amino acid sequence of SEQ ID NO:24. In some embodiments, a polypeptide comprises a V_{L} that has at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO:24. In some embodiments, a polypeptide comprises a V_{L} that has at least about 85% sequence identity to the amino acid sequence of SEQ ID NO:24. In some embodiments, a polypeptide comprises a V_{L} that has at least about 90% sequence identity to the amino acid sequence of SEQ ID NO: 24.

In some embodiments, a polypeptide comprises a V_{L} that comprises at least one amino acid substitution (e.g., at least one conservative substitution such as highly conservative substitution) relative to the amino acid sequence of SEQ ID NO:24. For example, the number of amino acid substitutions can be at least about: 2, 3, 4, 5, 6, 7, 8, 9, 10, 11, 12, 13, 14, 15, 16, 17, 18, 19, or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11, or 9-11. In some embodiments, a polypeptide comprises a V_{L} that comprises about 1-10 amino acid substitutions, relative to the amino acid sequence of SEQ ID NO:24. In some embodiments, the at least one amino acid substitution replaces only a light chain complementarity determining region 1 (LCDR1), a light chain complementarity determining region 2 (LCDR2), and/or a light chain complementarity determining region 3 (LCDR3) residue, of SEQ ID NO:24. In some embodiments, the at least one amino acid substitution replaces only a non-CDR residue (e.g., within a framework region), of SEQ ID NO:24.

In some embodiments, an amino acid substitution is a conservative substitution.

In some embodiments, an amino acid substitution is a highly conservative substitution.

In some embodiments, a polypeptide comprises a V_{L} that has 100% sequence identity to the amino acid sequence of SEQ ID NO:24. In some embodiments, a polypeptide comprises a V_{L} that comprises the amino acid sequence of SEQ ID NO:24.

In some embodiments, a polypeptide comprises a V_{L} that has at least about 70% sequence identity to the amino acid sequence of any one or more of SEQ ID NOs: 25-39. The sequences identified as SEQ ID NOs: 25-39 are shown in Table 2, which correspond to human V_{L} domains. In some embodiments, a polypeptide comprises a V_{L} that has at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of any one or more of SEQ ID NOs: 25-39. In some embodiments, a V_{L} has at least about 85% sequence identity to the amino acid sequence of any one or more of SEQ ID NOs: 25-39. In some embodiments, a polypeptide comprises a V_{L} that has at least about 90% sequence identity to the amino acid sequence of any one or more of SEQ ID NOs: 25-39.

In some embodiments, a polypeptide comprises a V_{L} that comprises at least one amino acid substitution relative to the amino acid sequence of any one or more of SEQ ID NOs: 25-39. For example, the number of amino acid substitutions can be at least about: 2, 3, 4, 5, 6, 7, 8, 9, 10, 11, 12, 13, 14, 15, 16, 17, 18, 19, or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11, or 9-11. In some embodiments, a polypeptide comprises a V_{L} that comprises about 1-10 amino acid substitutions, relative to the amino acid sequence of any one or more of SEQ ID NOs: 25-39.

In some embodiments, a polypeptide comprises a V_{L} that has 100% sequence identity to the amino acid sequence of any one of SEQ ID NO:25-39. In some embodiments, a polypeptide comprises a V_{L} that comprises the amino acid sequence of any one of SEQ ID NO: 25-39.

In some embodiments, a polypeptide comprises a V_{L} that has at least about 70% sequence identity to the amino acid sequence of SEQ ID NO:37. In some embodiments, a polypeptide comprises a V_{L} that has at least about: 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO:37. In some embodiments, a V_{L} has at least about 85% sequence identity to the amino acid sequence of SEQ ID NO:37. In some embodiments, a polypeptide comprises a V_{L} that has at least about 90% sequence identity to the amino acid sequence of SEQ ID NO:37.

In some embodiments, a polypeptide comprises a V_{L} that comprises at least one amino acid substitution relative to the amino acid sequence of SEQ ID NO:37. For example, the number of amino acid substitutions can be at least about: 2, 3, 4, 5, 6, 7, 8, 9, 10, 11, 12, 13, 14, 15, 16, 17, 18, 19, or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11, or 9-11. In some embodiments, a polypeptide comprises a V_{L} that comprises about 1-10 amino acid substitutions, relative to the amino acid sequence of SEQ ID NO:37.

In some embodiments, a polypeptide comprises a V_{L} that has 100% sequence identity to the amino acid sequence of SEQ ID NO:37. In some embodiments, a polypeptide comprises a V_{L} that comprises the amino acid sequence of SEQ ID NO:37.

In some embodiments, a polypeptide comprises a V_{H} and a V_{L}. In some embodiments, a polypeptide comprises a V_{H} and V_{L} that are humanized, contain human framework regions, or both.

In some embodiments, a polypeptide comprises:
a) a V_{H} having less than 100% sequence identity to the amino acid sequence of SEQ ID NO: 5;
b) a V_{L} having less than 100% sequence identity to the amino acid sequence of SEQ ID NO: 24; or both a) and b).

In some embodiments, a polypeptide comprises:
a) a V_{H} having less than 100% sequence identity to the amino acid sequence of SEQ ID NO: 5; and
b) a V_{L} having less than 100% sequence identity to the amino acid sequence of SEQ ID NO: 24.

In some embodiments, a polypeptide comprises:
a) a V_{H} that has at least about 55% (e.g., at least about: 60, 65, 70, 75, 80, 85, 90, 95, 98, or 99%) sequence identity to the amino acid sequence of SEQ ID NO:5;
b) a V_{L} that has at least about 55% (e.g., at least about: 60, 65, 70, 75, 80, 85, 90, 95, 98, or 99%) sequence identity to the amino acid sequence of SEQ ID NO:24; or both a) and b),
   wherein the polypeptide does not comprise all 6 CDRs of an antibody comprising a V_{H} amino acid sequence of SEQ ID NO:5 and a V_{L} amino acid sequence of SEQ ID NO:24.

In some embodiments, a polypeptide comprises:
a) a V_{H} that has at least about 55% (e.g., at least about: 60, 65, 70, 75, 80, 85, 90, 95, 98, or 99%) sequence identity to the amino acid sequence of SEQ ID NO:5; and
b) a V_{L} that has at least about 55% (e.g., at least about: 60, 65, 70, 75, 80, 85, 90, 95, 98, or 99%) sequence identity to the amino acid sequence of SEQ ID NO:24,
   wherein the polypeptide does not comprise all 6 CDRs of an antibody comprising a V_{H} amino acid sequence of SEQ ID NO:5 and a V_{L} amino acid sequence of SEQ ID NO:24.

In some embodiments, a polypeptide comprises:
a) a V_{H} that comprises at least one amino acid substitution (e.g., at least one conservative substitution such as highly conservative substitution) relative to the amino acid sequence of SEQ ID NO:5;
b) a V_{L} that comprises at least one amino acid substitution (e.g., at least one conservative substitution such as highly conservative substitution) relative to the amino acid sequence of SEQ ID NO:24; or both a) and b).

In some embodiments, a polypeptide comprises:
a) a V_{H} that comprises at least one amino acid substitution (e.g., at least one conservative substitution such as highly conservative amino acid substitution) relative to the amino acid sequence of SEQ ID NO:5; and
b) a V_{L} that comprises at least one amino acid substitution (e.g., at least one conservative substitution such as highly conservative amino acid substitution) relative to the amino acid sequence of SEQ ID NO:24.

In some embodiments, a polypeptide comprises:
a) a V_{H} that comprises at least one amino acid substitution (e.g., at least one conservative substitution such as highly conservative amino acid substitution) of a HCDR1, a HCDR2 and/or a HCDR3 residue, of SEQ ID NO:5;
b) a V_{L} that comprises at least one amino acid substitution (e.g., at least one conservative substitution such as highly conservative amino acid substitution) of a LCDR1, a LCDR2 and/or a LCDR3 residue, of SEQ ID NO:24; or both a) and b).

In some embodiments, a polypeptide comprises:
a) a V_{H} that comprises at least one amino acid substitution (e.g., at least one conservative substitution such as highly conservative amino acid substitution) of a HCDR1, a HCDR2 and/or a HCDR3 residue, of SEQ ID NO:5; and
b) a V_{L} that comprises at least one amino acid substitution (e.g., at least one conservative substitution such as highly conservative amino acid substitution) of a LCDR1, a LCDR2 and/or a LCDR3 residue, of SEQ ID NO:24.

In some embodiments, a polypeptide comprises:
a) a V_{H} that comprises at least one amino acid substitution (e.g., at least one conservative substitution such as highly conservative amino acid substitution) of a non-CDR residue (e.g., within a framework region), of SEQ ID NO:5;
b) a V_{L} that comprises at least one amino acid substitution (e.g., at least one conservative substitution such as highly conservative amino acid substitution) of a non-CDR residue (e.g., within a framework region), of SEQ ID NO:24; or both a) and b).

In some embodiments, a polypeptide comprises:
a) a V_{H} that comprises at least one amino acid substitution (e.g., at least one conservative substitution such as highly conservative amino acid substitution) of a non-CDR residue (e.g., within a framework region), of SEQ ID NO:5; and
b) a V_{L} that comprises at least one amino acid substitution (e.g., at least one conservative substitution such as highly conservative amino acid substitution) of a non-CDR residue (e.g., within a framework region), of SEQ ID NO:24.

In some embodiments, a polypeptide comprises:
a) a V_{H} that has at least about 70% (e.g., at least about: 75, 80, 85, 90, 95, 98, or 99%) sequence identity to the amino acid sequence of any one or more of SEQ ID NOs: 6-20;
b) a V_{L} that has at least about 70% (e.g., at least about: 75, 80, 85, 90, 95, 98, or 99%) sequence identity to the amino acid sequence of any one or more of SEQ ID NOs: 25-39; or both a) and b).

In some embodiments, a polypeptide comprises:
a) a V_{H} that has at least about 70% (e.g., at least about: 75, 80, 85, 90, 95, 98, or 99%) sequence identity to the amino acid sequence of any one or more of SEQ ID NOs: 6-20; and
b) a V_{L} that has at least about 70% (e.g., at least about: 75, 80, 85, 90, 95, 98, or 99%) sequence identity to the amino acid sequence of any one or more of SEQ ID NOs: 25-39.

In some embodiments, a polypeptide comprises:
a) a V_{H} that comprises at least one amino acid substitution (e.g., at least one conservative substitution such as highly conservative amino acid substitution) relative to the amino acid sequence of any one or more of SEQ ID NOs: 6-20;
b) a V_{L} that comprises at least one amino acid substitution (e.g., at least one conservative substitution such as highly conservative amino acid substitution) relative to the amino acid sequence of any one or more of SEQ ID NOs: 25-39; or
   both a) and b).

In some embodiments, a polypeptide comprises:
a) a V_{H} that comprises at least one amino acid substitution (e.g., at least one conservative substitution such as highly conservative amino acid substitution) relative to the amino acid sequence of any one or more of SEQ ID NOs: 6-20; and
b) a V_{L} that comprises at least one amino acid substitution (e.g., at least one conservative substitution such as highly conservative amino acid substitution) relative to the amino acid sequence of any one or more of SEQ ID NOs: 25-39.

In some embodiments, a polypeptide comprises:
a) a V_{H} that comprises at least one amino acid substitution (e.g., at least one conservative substitution such as highly conservative amino acid substitution) of a HCDR1, a HCDR2 and/or a HCDR3 residue, of any one or more of SEQ ID NOs: 6-20;
b) a V_{L} that comprises at least one amino acid substitution (e.g., at least one conservative substitution such as highly conservative amino acid substitution) of a LCDR1, a LCDR2 and/or a LCDR3 residue, of any one or more of SEQ ID NOs: 25-39; or both a) and b).

In some embodiments, a polypeptide comprises:
a) a V_{H} that comprises at least one amino acid substitution (e.g., at least one conservative substitution such as highly conservative amino acid substitution) of a HCDR1, a HCDR2 and/or a HCDR3 residue, of any one or more of SEQ ID NOs: 6-20; and
b) a V_{L} that comprises at least one amino acid substitution (e.g., at least one conservative substitution such as highly conservative amino acid substitution) of a LCDR1, a LCDR2 and/or a LCDR3 residue, of any one or more of SEQ ID NOs: 25-39.

In some embodiments, a polypeptide comprises:
a) a V_{H} that comprises at least one amino acid substitution (e.g., at least one conservative substitution such as highly conservative amino acid substitution) of a non-CDR residue (e.g., within a framework region), of any one or more of SEQ ID NOs: 6-20;
b) a V_{L} that comprises at least one amino acid substitution (e.g., at least one conservative substitution such as highly conservative amino acid substitution) of a non-CDR residue (e.g., within a framework region), of any one or more of SEQ ID NOs: 25-39; or both a) and b).

In some embodiments, a polypeptide comprises:
a) a V_{H} that comprises at least one amino acid substitution (e.g., at least one conservative substitution such as highly conservative amino acid substitution) of a non-CDR residue (e.g., within a framework region), of any one or more of SEQ ID NOs: 6-20; and
b) a V_{L} that comprises at least one amino acid substitution (e.g., at least one conservative substitution such as highly conservative amino acid substitution) of a non-CDR residue (e.g., within a framework region), of any one or more of SEQ ID NOs: 25-39.

In some embodiments, a polypeptide disclosed herein comprises:
a) a V_{H} comprising the amino acid sequence of any one of SEQ ID NOs: 6-20; and
b) a V_{L} comprising the amino acid sequence of any one of SEQ ID NO:25-39.

In some embodiments, a polypeptide comprises:
a) a V_{H} comprising the amino acid sequence of any one of SEQ ID NOs: 6-20; and
b) a V_{L} comprising the amino acid sequence of SEQ ID NO:24.

In some embodiments, a polypeptide comprises:
a) a V_{H} comprising the amino acid sequence of SEQ ID NO:5; and
b) a V_{L} comprising the amino acid sequence of any one of SEQ ID NO:25-39.

In some embodiments, a polypeptide comprises:
a) a V_{H} that has at least about 70% sequence identity to the amino acid sequence of SEQ ID NO:18;
b) a V_{L} that has at least about 70% sequence identity to the amino acid sequence of SEQ ID NO:37; or
   both a) and b).

In some embodiments, a polypeptide comprises:
a) a V_{H} that has at least about 70% sequence identity to the amino acid sequence of SEQ ID NO:18; and
b) a V_{L} that has at least about 70% sequence identity to the amino acid sequence of SEQ ID NO:37.

In some embodiments, a polypeptide comprises:
a) a V_{H} that comprises at least one amino acid substitution (e.g., at least one conservative substitution such as highly conservative amino acid substitution) relative to the amino acid sequence of SEQ ID NO:18;
b) a V_{L} that comprises at least one amino acid substitution (e.g., at least one conservative substitution such as highly conservative amino acid substitution) relative to the amino acid sequence of SEQ ID NO:37; or both a) and b).

In some embodiments, a polypeptide comprises:
a) a V_{H} that comprises at least one amino acid substitution (e.g., at least one conservative substitution such as highly conservative amino acid substitution) relative to the amino acid sequence of SEQ ID NO:18; and
b) a V_{L} that comprises at least one amino acid substitution (e.g., at least one conservative substitution such as highly conservative amino acid substitution) relative to the amino acid sequence of SEQ ID NO:37.

In some embodiments, a polypeptide comprises:
a) a V_{H} that comprises at least one amino acid substitution (e.g., at least one conservative substitution such as highly conservative amino acid substitution) of a HCDR1, a HCDR2 and/or a HCDR3 residue, of SEQ ID NO:18;
b) a V_{L} that comprises at least one amino acid substitution (e.g., at least one conservative substitution such as highly conservative amino acid substitution) of a LCDR1, a LCDR2 and/or a LCDR3 residue, of SEQ ID NO:37; or both a) and b).

In some embodiments, a polypeptide comprises:
a) a V_{H} that comprises at least one amino acid substitution (e.g., at least one conservative substitution such as highly conservative amino acid substitution) of a HCDR1, a HCDR2 and/or a HCDR3 residue, of SEQ ID NO:18; and
b) a V_{L} that comprises at least one amino acid substitution (e.g., at least one conservative substitution such as highly conservative amino acid substitution) of a LCDR1, a LCDR2 and/or a LCDR3 residue, of SEQ ID NO:37.

In some embodiments, a polypeptide comprises:
a) a V_{H} that comprises at least one amino acid substitution (e.g., at least one conservative substitution such as highly conservative amino acid substitution) of a non-CDR residue (e.g., within a framework region), of SEQ ID NO:18;
b) a V_{L} that comprises at least one amino acid substitution (e.g., at least one conservative substitution such as highly conservative amino acid substitution) of a non-CDR residue (e.g., within a framework region), of SEQ ID NO:37; or
   both a) and b).

In some embodiments, a polypeptide comprises:
a) a V_{H} that comprises at least one amino acid substitution (e.g., at least one conservative substitution such as highly conservative amino acid substitution) of a non-CDR residue (e.g., within a framework region), of SEQ ID NO:18; and
b) a V_{L} that comprises at least one amino acid substitution (e.g., at least one conservative substitution such as highly conservative amino acid substitution) of a non-CDR residue (e.g., within a framework region), of SEQ ID NO:37.

In some embodiments, a polypeptide comprises:
a) a V_{H} comprising the amino acid sequence of SEQ ID NO:18; and
b) a V_{L} comprising the amino acid sequence of any one of SEQ ID NO:24-39.

In some embodiments, a polypeptide comprises:
a) a V_{H} comprising the amino acid sequence of any one of SEQ ID NOs: 5-20; and
b) a V_{L} comprising the amino acid sequence of SEQ ID NO:37.

In some embodiments, a polypeptide comprises:
a) a V_{H} comprising the amino acid sequence of SEQ ID NO:6; and
b) a V_{L} comprising the amino acid sequence of SEQ ID NO:25 (AB-1).

In some embodiments, a polypeptide comprises:
a) a V_{H} comprising the amino acid sequence of SEQ ID NO:7; and
b) a V_{L} comprising the amino acid sequence of SEQ ID NO:26 (AB-2).

In some embodiments, a polypeptide comprises:
a) a V_{H} comprising the amino acid sequence of SEQ ID NO:8; and
b) a V_{L} comprising the amino acid sequence of SEQ ID NO:27 (AB-3).

In some embodiments, a polypeptide comprises:
a) a V_{H} comprising the amino acid sequence of SEQ ID NO:9; and
b) a V_{L} comprising the amino acid sequence of SEQ ID NO:28 (AB-4).

In some embodiments, a polypeptide comprises:
a) a V_{H} comprising the amino acid sequence of SEQ ID NO:10; and
b) a V_{L} comprising the amino acid sequence of SEQ ID NO:29 (AB-5).

In some embodiments, a polypeptide comprises:
a) a V_{H} comprising the amino acid sequence of SEQ ID NO:11; and
b) a V_{L} comprising the amino acid sequence of SEQ ID NO:30 (AB-6).

In some embodiments, a polypeptide comprises:
a) a V_{H} comprising the amino acid sequence of SEQ ID NO:12; and
b) a V_{L} comprising the amino acid sequence of SEQ ID NO:31 (AB-7).

In some embodiments, a polypeptide comprises:
a) a V_{H} comprising the amino acid sequence of SEQ ID NO:13; and
b) a V_{L} comprising the amino acid sequence of SEQ ID NO:32 (AB-8).

In some embodiments, a polypeptide comprises:
a) a V_{H} comprising the amino acid sequence of SEQ ID NO:14; and
b) a V_{L} comprising the amino acid sequence of SEQ ID NO:33 (AB-9).

In some embodiments, a polypeptide comprises:
a) a V_{H} comprising the amino acid sequence of SEQ ID NO:15; and
b) a V_{L} comprising the amino acid sequence of SEQ ID NO:34 (AB-10).

In some embodiments, a polypeptide comprises:
a) a V_{H} comprising the amino acid sequence of SEQ ID NO:16; and
b) a V_{L} comprising the amino acid sequence of SEQ ID NO:35 (AB-11).

In some embodiments, a polypeptide comprises:
a) a V_{H} comprising the amino acid sequence of SEQ ID NO:17; and
b) a V_{L} comprising the amino acid sequence of SEQ ID NO:36 (AB-12).

In some embodiments, a polypeptide comprises:
a) a V_{H} comprising the amino acid sequence of SEQ ID NO:18; and
b) a V_{L} comprising the amino acid sequence of SEQ ID NO:37 (AB-13).

In some embodiments, a polypeptide comprises:
a) a V_{H} comprising the amino acid sequence of SEQ ID NO:19; and
b) a V_{L} comprising the amino acid sequence of SEQ ID NO:38 (AB-14).

In some embodiments, a polypeptide comprises:
a) a V_{H} comprising the amino acid sequence of SEQ ID NO:20; and
b) a V_{L} comprising the amino acid sequence of SEQ ID NO:39 (AB-15).

### Complementarity Determining Regions (CDRs)

A CDR (e.g., HCDR1, HCDR2, HCDR3, LCDR1, LCDR2 and/or LCDR3) can be a CDR defined by any art-recognized method for identifying CDR residues of an antibody, as described further herein (e.g., a CDR as defined by Kabat, a CDR as defined by Chothia, or a CDR as defined by IMGT).

In some embodiments, a polypeptide does not comprise all six CDRs of an antibody comprising a V_{H} amino acid sequence of SEQ ID NO:5 and a V_{L} amino acid sequence of SEQ ID NO: 24. In some embodiments, a polypeptide does not comprise all six sequences of SEQ ID NO: 43, SEQ ID NO:47, SEQ ID NO:55, SEQ ID NO:72, SEQ ID NO: 84 and SEQ ID NO:87. See Table 2 for SEQ ID NO:43, SEQ ID NO:47, SEQ ID NO:55, SEQ ID NO:72, SEQ ID NO: 84 and SEQ ID NO:87.

In some embodiments, a polypeptide comprises fewer than six (e.g., 1, 2, 3, 4, or 5) CDRs of an antibody comprising a V_{H} amino acid sequence of SEQ ID NO:5 and a V_{L} amino acid sequence of SEQ ID NO:24. In some embodiments, a polypeptide comprises 1, 2, 3, 4, or 5 CDRs selected from SEQ ID NO:43, SEQ ID NO:47, SEQ ID NO:55, SEQ ID NO:72, SEQ ID NO: 84 and SEQ ID NO:87.

In some embodiments, a polypeptide comprises all six CDRs of a specific polypeptide disclosed herein. In some embodiments, a polypeptide comprises fewer than six (e.g., 1, 2, 3, 4, or 5) of CDRs of a specific polypeptide disclosed herein.

In some embodiments, a polypeptide comprises:
a) a V_{H} amino acid sequence comprising a HCDR1, a HCDR2, and a HCDR3 that are substantially similar in amino acid sequence to a HCDR1, a HCDR2 and a HCDR3, respectively, of a Vu amino acid sequence set forth in any one of SEQ ID NOs: 6-20;
b) a V_{L} amino acid sequence comprising a LCDR1, a LCDR2 and a LCDR3 that are substantially similar in amino acid sequence to a LCDR1, a LCDR2 and a LCDR3, respectively, of a V_{L} amino acid sequence set forth in any one of SEQ ID NOs: 25-39; or
   both a) and b).

In some embodiments, a polypeptide comprises:
a) a V_{H} amino acid sequence comprising a HCDR1, a HCDR2, and a HCDR3 that are substantially similar in amino acid sequence to a HCDR1, a HCDR2 and a HCDR3, respectively, of a V_{H} amino acid sequence set forth in any one of SEQ ID NOs: 6-20; and
b) a V_{L} amino acid sequence comprising a LCDR1, a LCDR2 and a LCDR3 that are substantially similar in amino acid sequence to a LCDR1, a LCDR2 and a LCDR3, respectively, of a V_{L} amino acid sequence set forth in any one of SEQ ID NOs: 25-39.

See Tables 2-3 and FIGS. 2A-3B for SEQ ID NOs: 6-20 and 25-39, and non-limiting examples of corresponding HCDR1, HCDR2, HCDR3, LCDR1, LCDR2, and LCDR3 sequences.

In some embodiments, a polypeptide comprises:
a) a V_{H} amino acid sequence comprising a HCDR1, a HCDR2, and a HCDR3 that are substantially similar in amino acid sequence to a HCDR1, a HCDR2 and a HCDR3, respectively, of a V_{H} amino acid sequence of any one or more of SEQ ID NOs: 6-20; and
b) a V_{L} amino acid sequence comprising a LCDR1, a LCDR2, and a LCDR3 that are substantially similar in amino acid sequence to a LCDR1, a LCDR2 and a LCDR3, respectively, of a V_{L} amino acid sequence of any one or more of SEQ ID NOs: 25-39.

In some embodiments, a polypeptide comprises a HCDR1, a HCDR2, a HCDR3, a LCDR1, a LCDR2 and a LCDR3 that substantially preserve one or more functional properties of a HCDR1, a HCDR2, a HCDR3, a LCDR1, a LCDR2 and a LCDR3 of a polypeptide selected from any one of AB-1 to AB-15c (e.g., AB-13).

In some embodiments, a polypeptide comprises a HCDR1, a HCDR2, a HCDR3, a LCDR1, a LCDR2 and a LCDR3 comprising only one or more conservative substitutions (e.g., only one or more highly conservative substitutions), relative a HCDR1, a HCDR2, a HCDR3, a LCDR1, a LCDR2 and a LCDR3 of a polypeptide selected from any one of AB-1 to AB-15c (e.g., AB-13).

In some embodiments, a polypeptide comprises a HCDR1, a HCDR2, a HCDR3, a LCDR1, a LCDR2 and a LCDR3 comprising up to 1, 2, or 3 conservative substitutions (e.g., up to 1, 2, or 3 highly conservative substitutions), relative a HCDR1, a HCDR2, a HCDR3, a LCDR1, a LCDR2 and a LCDR3 of a polypeptide selected from any one of AB-1 to AB-15c (e.g., AB-13).

In some embodiments, a polypeptide disclosed herein comprises a HCDR1, a HCDR2, a HCDR3, a LCDR1, a LCDR2 and a LCDR3 having 100% sequence identity to a HCDR1, a HCDR2, a HCDR3, a LCDR1, a LCDR2 and a LCDR3 of a polypeptide selected from any one of AB-1 to AB-15c (e.g., AB-13).

In some embodiments, a polypeptide comprises a HCDR1, a HCDR2, a HCDR3, a LCDR1, a LCDR2 and a LCDR3, of an antibody comprising a V_{H}/V_{L} combination selected from:
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c); or
SEQ ID NO:20 and SEQ ID NO:39 (AB-15c).

In some embodiments, a polypeptide comprises:
a) a HCDR1 comprising at least one amino acid substitution relative to at least one amino acid sequence set forth in SEQ ID NOs: 43-45 (e.g., at least one amino acid sequence set forth in SEQ ID NO:44 or SEQ ID NO:45);
b) a HCDR2 comprising at least one amino acid substitution relative to at least one amino acid sequence set forth in SEQ ID NOs: 47-53 (e.g., at least one amino acid sequence set forth in SEQ ID NOs: 48-53);
c) a HCDR3 comprising at least one amino acid substitution relative to at least one amino acid sequence set forth in SEQ ID NOs: 55-70 (e.g., at least one amino acid sequence set forth in SEQ ID NOs: 56-70);
d) a LCDR1 comprising at least one amino acid substitution relative to the amino acid sequence set forth in SEQ ID NOs: 72-82 (e.g., at least one amino acid sequence set forth in SEQ ID NOs: 73-82);
e) a LCDR2 comprising at least one amino acid substitution relative to at least one amino acid sequence set forth in SEQ ID NO:84 or SEQ ID NO:85 (e.g., SEQ ID NO: 85);
f) a LCDR3 comprising at least one amino acid substitution relative to at least one amino acid sequence set forth in SEQ ID NOs: 87-97 (e.g., at least one amino acid sequence set forth in SEQ ID NOs: 88-97);
   or any combination of the foregoing.

### Paratopes

Amino acid residues of a paratope contribute to an antibody's interaction with an epitope of its target protein. An interaction can be a hydrogen bond, a salt bridge, a van der Waals interaction, an electrostatic interaction, a hydrophobic interaction, pi-interaction effects, an ionic bond, and/or any combination thereof. An interaction can be direct, or indirect, e.g., via a coordinated intermediate molecule, such as an ion or water. The residues of a paratope, in some embodiments, comprise only residues that are part of a defined CDR. In some embodiments, the residues of a paratope further comprise one or more residues that are not part of a defined CDR (e.g., residues within a defined framework region).

In some embodiments, a paratope is oriented less than about 5.0 angstroms from an epitope on a target antigen when a polypeptide is bound to the target antigen, e.g., less than about: 4.5, 4.0, 3.5, 3.0, 2.5, 2.4, 2.3, 2.2, 2.1, 2.0, 1.9, 1.8, 1.7, 1.6, 1.5, 1.4, 1.3, 1.2, 1.1, 1.0 or 0.9 angstroms, or about: 0.9-5.0, 0.9-4.8. 1.0-5, 1.0-4.5, 1.0-4.0, 1.0-3.5, 1.1-3.5, 1.1-3.0, 1.2-3.0, 1.2-2.5, 1.3-2.5, 1.3-2.4, 1.4-2.4, 1.4-2.3, 1.5-2.3, 1.5-2.2, 1.6-2.2, 1.6-2.1, 1.7-2.1, 1.7-2.0 or 1.8-2.0 angstroms, from the epitope. In some embodiments, less than all of the amino acid residues constituting a paratope (e.g., about 40%, about 50%, about 60%, about 70%, about 80%, about 90%, about 95%, about 96%, about 97%, about 98%, or about 99% of the amino acid residues) in the paratope are oriented less than about 5.0 angstroms from an epitope on a target antigen when a polypeptide is bound to the target antigen.

In some embodiments, a polypeptide comprising a paratope disclosed herein comprises a V_{H} and a V_{L}. In some embodiments, paratope residues are contained within the V_{H} and V_{L} of a polypeptide.

In some embodiments, a polypeptide comprises a paratope that differs from a paratope of an antibody comprising a V_{H}/V_{L} combination of SEQ ID NO:5/SEQ ID NO:24.

In some embodiments, a polypeptide comprises a paratope that differs from a paratope of an antibody comprising a V_{H}/V_{L} combination of SEQ ID NO:5/SEQ ID NO:24, by substitution (e.g., conservative substitution such as highly conservative substitution) of from 1 to 3 (e.g., 1, 2 or 3) residues.

In some embodiments, a polypeptide comprises a paratope that is substantially similar (e.g., having at least about 90% sequence identity) to a paratope of an antibody comprising a V_{H}/V_{L} combination of SEQ ID NO:5/SEQ ID NO:24. In some embodiments, a polypeptide comprises a paratope that is substantially similar (e.g., having at least about 90% sequence identity) to, and substantially preserves one or more functional properties of, a paratope of an antibody comprising a V_{H}/V_{L} combination of SEQ ID NO:5/SEQ ID NO:24.

In some embodiments, a polypeptide comprises a paratope that is identical to a paratope of an antibody comprising a V_{H}/V_{L} combination of SEQ ID NO:5/SEQ ID NO:24

In some embodiments, a polypeptide comprises a paratope that differs from a paratope of an antibody comprising a V_{H}/V_{L} combination selected from: SEQ ID NO:6/SEQ ID NO: 25 (AB-1), SEQ ID NO:7/SEQ ID NO:26 (AB-2), SEQ ID NO:8/SEQ ID NO:27 (AB-3), SEQ ID NO:9/SEQ ID NO:28 (AB-4), SEQ ID NO: 10/SEQ ID NO:29 (AB-5), SEQ ID NO: 11/SEQ ID NO:30 (AB-6), SEQ ID NO:12/SEQ ID NO:31 (AB-7), SEQ ID NO:13/SEQ ID NO: 32 (AB-8), SEQ ID NO:14/SEQ ID NO:33 (AB-9), SEQ ID NO:15/SEQ ID NO:34 (AB-10), SEQ ID NO:16/SEQ ID NO:35 (AB-11), SEQ ID NO:17/SEQ ID NO:36 (AB-12), SEQ ID NO: 18/SEQ ID NO:37 (AB-13), SEQ ID NO:19/SEQ ID NO:38 (AB-14c), or SEQ ID NO: 20/SEQ ID NO:39 (AB-15c).

See Table 2 for SEQ ID NOs: 6-20 and SEQ ID NOs: 25-39, and FIGS. 2A-3B for the paratope residues of antibodies comprising V_{H} sequences set forth in SEQ ID NOs: 6-20 and V_{L} sequences set forth in SEQ ID Nos: 25-39.

In some embodiments, a polypeptide comprises a paratope that differs from a paratope of an antibody comprising a V_{H}/V_{L} combination selected from: SEQ ID NO:6/SEQ ID NO: 25 (AB-1), SEQ ID NO:7/SEQ ID NO:26 (AB-2), SEQ ID NO:8/SEQ ID NO:27 (AB-3), SEQ ID NO:9/SEQ ID NO:28 (AB-4), SEQ ID NO:10/SEQ ID NO:29 (AB-5), SEQ ID NO: 11/SEQ ID NO:30 (AB-6), SEQ ID NO:12/SEQ ID NO:31 (AB-7), SEQ ID NO:13/SEQ ID NO: 32 (AB-8), SEQ ID NO:14/SEQ ID NO:33 (AB-9), SEQ ID NO:15/SEQ ID NO:34 (AB-10), SEQ ID NO:16/SEQ ID NO:35 (AB-11), SEQ ID NO:17/SEQ ID NO:36 (AB-12), SEQ ID NO: 18/SEQ ID NO:37 (AB-13), SEQ ID NO:19/SEQ ID NO:38 (AB-14c), or SEQ ID NO: 20/SEQ ID NO:39 (AB-15c), by substitution (e.g., conservative substitution such as highly conservative substitution) of from 1 to 3 (e.g., 1, 2 or 3) residues.

In some embodiments, a polypeptide comprises a paratope that is substantially similar (e.g., having at least about 90% sequence identity) to a paratope of an antibody comprising a V_{H}/V_{L} combination selected from:
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c); or
SEQ ID NO:20 and SEQ ID NO:39 (AB-15c); or
any combination of the foregoing.

In some embodiments, a polypeptide comprises a paratope that is substantially similar (e.g., having at least about 90% sequence identity) to, and substantially preserves one or more functional properties of, a paratope of an antibody comprising a V_{H}/V_{L} combination selected from:
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c); or
SEQ ID NO:20 and SEQ ID NO:39 (AB-15c); or
any combination of the foregoing.

In some embodiments, a polypeptide comprises a paratope comprising only one or more conservative substitutions (e.g., only one or more highly conservative substitutions) relative a paratope of an antibody comprising a V_{H}/V_{L} combination selected from:
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c); or
SEQ ID NO:20 and SEQ ID NO:39 (AB-15c); or any combination of the foregoing.

In some embodiments, a polypeptide comprises a paratope comprising up to 1, 2, 3, 4, 5, 6, 7, 8, 9, 10, 11, 12, 13, 14 or 15 conservative substitutions (e.g., up to 1, 2, 3, 4, 5, 6, 7, 8, 9, 10, 11, 12, 13, 14 or 15 highly conservative substitutions), relative a paratope of an antibody comprising a V_{H}/V_{L} combination selected from:
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c); or
SEQ ID NO:20 and SEQ ID NO:39 (AB-15c); or
any combination of the foregoing.

In some embodiments, a polypeptide comprises a paratope that has 100% sequence identity to a paratope of an antibody comprising a V_{H}/V_{L} combination selected from:
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c); or
SEQ ID NO:20 and SEQ ID NO:39 (AB-15c); or
any combination of the foregoing.

In some embodiments, a polypeptide comprises a paratope that differs from a paratope of an antibody comprising a V_{H}/V_{L} combination of SEQ ID NO:18/SEQ ID NO:37 (AB-13).

In some embodiments, a polypeptide comprises a paratope that differs from a paratope of an antibody comprising a V_{H}/V_{L} combination of SEQ ID NO:18/SEQ ID NO:37 (AB-13), by substitution (e.g., conservative substitution such as highly conservative substitution) of from 1 to 3 (e.g., 1, 2 or 3) residues.

In some embodiments, a polypeptide comprises a paratope that is substantially similar (e.g., having at least about 90% sequence identity) to a paratope of an antibody comprising a V_{H}/V_{L} combination of SEQ ID NO:18/SEQ ID NO:37 (AB-13).

In some embodiments, a polypeptide comprises a paratope that is substantially similar (e.g., having at least about 90% sequence identity) to, and substantially preserves one or more functional properties of, a paratope of an antibody comprising a V_{H}/V_{L} combination of SEQ ID NO: 18/SEQ ID NO:37 (AB-13).

In some embodiments, a polypeptide comprises a paratope comprising only one or more conservative substitutions (e.g., only one or more highly conservative substitutions) relative a paratope of an antibody comprising a V_{H}/V_{L} of SEQ ID NO:18/SEQ ID NO:37 (AB-13).

In some embodiments, a polypeptide comprises a paratope comprising up to 1, 2, 3, 4, 5, 6, 7, 8, 9, 10, 11, 12, 13, 14 or 15 conservative substitutions (e.g., up to 1, 2, 3, 4, 5, 6, 7, 8, 9, 10, 11, 12, 13, 14 or 15 highly conservative substitutions), relative a paratope of an antibody comprising a V_{H}/V_{L} combination of SEQ ID NO:18/SEQ ID NO:37 (AB-13).

In some embodiments, a polypeptide comprises a paratope that has 100% sequence identity to a paratope of an antibody comprising a V_{H}/V_{L} combination of SEQ ID NO:18/SEQ ID NO: 37 (AB-13).

In some embodiments, a polypeptide comprises a paratope comprising amino acid residues corresponding to each of X₂ (position 30), A33, T35, S50, I51, S52, G53, S54, X₄ (position 57), Y59, D99, X₇ (position 100), X₉ (position 102), X₁₀ (position 103), X₁₁ (position 104), X₁₂ (position 105), X₁₃ (position 106), P107, and X₁₄ (position 108) of SEQ ID NO:4, and Y31, S32, Y37, X₂₁ (position 96), L97, X₂₂ (position 98), X₂₃ (position T99), and Y101 of SEQ ID NO: 23, or a subset thereof.

### Consensus Sequences

In some embodiments, a polypeptide comprises a V_{H} comprising the amino acid sequence of SEQ ID NO:4, wherein:
X₁ is not T;
X₂ is not R;
X₃ is not G;
X₄ is not N;
X₅ is not T;
X₆ is not K;
X₇ is not R;
X₈ is not L;
X₉ is not S;
X₁₀ is not I;
X₁₁ is not T;
X₁₂ is not I;
X₁₃ is not R;
X₁₄ is not R;
X₁₅ is not Y; or
X₁₆ is not V;
or any combination of the foregoing.

The sequence identified as SEQ ID NO:4 is shown in Table 2, which is a consensus V_{H} sequence for SEQ ID Nos: 5-20 herein.

In some embodiments:
X₁ is T or N;
X₂ is R, S or K;
X₃ is G, S, D or T;
X₄ is N, T or K;
X₅ is T, K or I;
X₆ is K or R;
X₇ is R or A;
X₈ is L, W or Y;
X₉ is S or T;
X₁₀ is I, A, K, T, S or V;
X₁₁ is T, A, K, S or R;
X₁₂ is I, V, A or T;
X₁₃ is R or K;
X₁₄ is R, Y, E, I, N, S, V or K;
X₁₅ is Y, V or R; or
X₁₆ is V or I,
or any combination of the foregoing.

In some embodiments:
X₁ is N;
X₂ is S or K;
X₃ is S, D or T;
X₄ is T or K;
X₅ is K or I;
X₆ is R;
X₇ is A;
X₈ is W or Y;
X₉ is T;
X₁₀ is A, K, T, S or V;
X₁₁ is A, K, S or R;
X₁₂ is V, A or T;
X₁₃ is K;
X₁₄ is Y, E, I, N, S, V or K;
X₁₅ is V or R; or
X₁₆ is I;
or any combination of the foregoing.

In some embodiments, X₁ is not T. In some embodiments, X₁ is T or N. In some embodiments, X₁ is T. In some embodiments, X₁ is N.

In some embodiments, X₂ is not R. In some embodiments, X₂ is R, S or K. In some embodiments, X₂ is S or K. In some embodiments, X₂ is R. In some embodiments, X₂ is S. In some embodiments, X₂ is K.

In some embodiments, X₃ is not G. In some embodiments, X₃ is G, S, D or T. In some embodiments, X₃ is S, D or T. In some embodiments, X₃ is G. In some embodiments, X₃ is S. In some embodiments, X₃ is D. In some embodiments, X₃ is T.

In some embodiments, X₄ is not N. In some embodiments, X₄ is N, T or K. In some embodiments, X₄ is T or K. In some embodiments, X₄ is N. In some embodiments, X₄ is T. In some embodiments, X₄ is K.

In some embodiments, X₅ is not T. In some embodiments, X₅ is T, K or I. In some embodiments, X₅ is K or I. In some embodiments, X₅ is T. In some embodiments, X₅ is K. In some embodiments, X₅ is I.

In some embodiments, X₆ is not K. In some embodiments, X₆ is K or R. In some embodiments, X₆ is K. In some embodiments, X₆ is R.

In some embodiments, X₇ is not R. In some embodiments, X₇ is R or A. In some embodiments, X₇ is R. In some embodiments, X₇ is A.

In some embodiments, X₈ is not L. In some embodiments, X₈ is L, W or Y. In some embodiments, X₈ is W or Y. In some embodiments, X₈ is L. In some embodiments, X₈ is W. In some embodiments, X₈ is Y.

In some embodiments, X₉ is not S. In some embodiments, X₉ is S or T. In some embodiments, X₉ is S. In some embodiments, X₉ is T.

In some embodiments, X₁₀ is not I. In some embodiments, X₁₀ is I, A, K, T, S or V. In some embodiments, X₁₀ is A, K, T, S or V. In some embodiments, X₁₀ is I. In some embodiments, X₁₀ is A. In some embodiments, X₁₀ is K. In some embodiments, X₁₀ is T. In some embodiments, X₁₀ is S. In some embodiments, X₁₀ is V.

In some embodiments, X₁₁ is not T. In some embodiments, X₁₁ is T, A, K, S or R. In some embodiments, X₁₁ is A, K, S or R. In some embodiments, X₁₁ is T. In some embodiments, X₁₁ is A. In some embodiments, X₁₁ is K. In some embodiments, X₁₁ is S. In some embodiments, X₁₁ is R.

In some embodiments, X₁₂ is not I. In some embodiments, X₁₂ is I, V, A or T. In some embodiments, X₁₂ is V, A or T. In some embodiments, X₁₂ is I. In some embodiments, X₁₂ is V. In some embodiments, X₁₂ is A. In some embodiments, X₁₂ is T.

In some embodiments, X₁₃ is not R. In some embodiments, X₁₃ is R or K. In some embodiments, X₁₃ is R. In some embodiments, X₁₃ is K.

In some embodiments, X₁₄ is not R. In some embodiments, X₁₄ is R, Y, E, I, N, S, V or K. In some embodiments, X₁₄ is Y, E, I, N, S, V or K. In some embodiments, X₁₄ is R. In some embodiments, X₁₄ is Y. In some embodiments, X₁₄ is E. In some embodiments, X₁₄ is I. In some embodiments, X₁₄ is N. In some embodiments, X₁₄ is S. In some embodiments, X₁₄ is V. In some embodiments, X₁₄ is K.

In some embodiments, X₁₅ is not Y. In some embodiments, X₁₅ is Y, V or R. In some embodiments, X₁₅ is V or R. In some embodiments, X₁₅ is Y. In some embodiments, X₁₅ is V. In some embodiments, X₁₅ is R.

In some embodiments, X₁₆ is not V. In some embodiments, X₁₆ is V or I. In some embodiments, X₁₆ is V. In some embodiments, X₁₆ is I.

In some embodiments, a polypeptide comprises a V_{L} comprising the amino acid sequence of SEQ ID NO:23, wherein:
X₁₇ is not L;
X₁₈ is not I;
X₁₉ is not Y;
X₂₀ is not G;
X₂₁ is not A;
X₂₂ is not Q; or
X₂₃ is not T;
or any combination of the foregoing.

The sequence identified as SEQ ID NO:23 is shown in Table 2, which is a consensus V_{L} sequence for SEQ ID Nos: 24-39 herein.

In some embodiments:
X₁₇ is L or D;
X₁₈ is I, Y, S, T, E, L, V, Q, H or R;
X₁₉ is Y or K;
X₂₀ is G or A;
X₂₁ is A, S, T or D;
X₂₂ is Q, S, K, T, F, H or Y; or
X₂₃ is T or H;
or any combination of the foregoing.

In some embodiments:
X₁₇ is D;
X₁₈ is Y, S, T, E, L, V, Q, H or R;
X₁₉ is K;
X₂₀ is A;
X₂₁ is S, T or D;
X₂₂ is S, K, T, F, H or Y; or
X₂₃ is H;
or any combination of the foregoing.

In some embodiments, X₁₇ is not L. In some embodiments, X₁₇ is L or D. In some embodiments, X₁₇ is L. In some embodiments, X₁₇ is D.

In some embodiments, X₁₈ is not I. In some embodiments, X₁₈ is I, Y, S, T, E, L, V, Q, H or R. In some embodiments, X₁₈ is Y, S, T, E, L, V, Q, H or R. In some embodiments, X₁₈ is Y. In some embodiments, X₁₈ is S. In some embodiments, X₁₈ is T. In some embodiments, X₁₈ is E. In some embodiments, X₁₈ is L. In some embodiments, X₁₈ is V. In some embodiments, X₁₈ is Q. In some embodiments, X₁₈ is H. In some embodiments, X₁₈ is R.

In some embodiments, X₁₉ is not Y. In some embodiments, X₁₉ is Y or K. In some embodiments, X₁₉ is Y. In some embodiments, X₁₉ is K.

In some embodiments, X₂₀ is not G. In some embodiments, X₂₀ is G or A. In some embodiments, X₂₀ is G. In some embodiments, X₂₀ is A.

In some embodiments, X₂₁ is not A. In some embodiments, X₂₁ is A, S, T or D. In some embodiments, X₂₁ is S, T or D. In some embodiments, X₂₁ is S. In some embodiments, X₂₁ is T. In some embodiments, X₂₁ is D.

In some embodiments, X₂₂ is not Q. In some embodiments, X₂₂ is Q, S, K, T, F, H or Y. In some embodiments, X₂₂ is S, K, T, F, H or Y. In some embodiments, X₂₂ is S. In some embodiments, X₂₂ is K. In some embodiments, X₂₂ is T. In some embodiments, X₂₂ is F. In some embodiments, X₂₂ is H. In some embodiments, X₂₂ is Y.

In some embodiments,
a) X₁ is T, X₂ is S, X₃ is G, X₄ is N, X₅ is T, X₆ is K, X₇ is A, X₈ is L, X₉ is T, X₁₀ is I, X₁₁ is A, X₁₂ is I, X₁₃ is R, X₁₄ is Y, X₁₅ is Y, X₁₆ is V, X₁₇ is D, X₁₈ is Y, X₁₉ is Y, X₂₀ is A, X₂₁ is S, X₂₂ is S, X₂₃ is T, or a combination thereof (AB-1);
b) X₁ is T, X₂ is R, X₃ is S, X₄ is N, X₅ is T, X₆ is R, X₇ is R, X₈ is L, X₉ is T, X₁₀ is A, X₁₁ is A, X₁₂ is V, X₁₃ is R, X₁₄ is Y, X₁₅ is Y, X₁₆ is V, X₁₇ is D, X₁₈ is S, X₁₉ is Y, X₂₀ is A, X₂₁ is S, X₂₂ is Q, X₂₃ is T, or a combination thereof (AB-2);
c) X₁ is T, X₂ is R, X₃ is G, X₄ is N, X₅ is T, X₆ is K, X₇ is R, X₈ is L, X₉ is S, X₁₀ is K, X₁₁ is T, X₁₂ is I, X₁₃ is R, X₁₄ is Y, X₁₅ is Y, X₁₆ is V, X₁₇ is L, X₁₈ is T, X₁₉ is Y, X₂₀ is G, X₂₁ is S, X₂₂ is S, X₂₃ is T, or a combination thereof (AB-3);
d) X₁ is T, X₂ is S, X₃ is G, X₄ is N, X₅ is T, X₆ is R, X₇ is R, X₈ is L, X₉ is T, X₁₀ is I, X₁₁ is T, X₁₂ is I, X₁₃ is R, X₁₄ is R, X₁₅ is Y, X₁₆ is V, X₁₇ is L, X₁₈ is T, X₁₉ is Y, X₂₀ is A, X₂₁ is A, X₂₂ is S, X₂₃ is T, or a combination thereof (AB-4);
e) X₁ is T, X₂ is S, X₃ is S, X₄ is N, X₅ is T, X₆ is K, X₇ is R, X₈ is L, X₉ is T, X₁₀ is I, X₁₁ is A, X₁₂ is I, X₁₃ is R, X₁₄ is Y, X₁₅ is Y, X₁₆ is V, X₁₇ is D, X₁₈ is I, X₁₉ is Y, X₂₀ is A, X₂₁ is A, X₂₂ is Q, X₂₃ is T, or a combination thereof (AB-5);
f) X₁ is T, X₂ is R, X₃ is S, X₄ is N, X₅ is T, X₆ is K, X₇ is R, X₈ is L, X₉ is T, X₁₀ is T, X₁₁ is K, X₁₂ is V, X₁₃ is R, X₁₄ is E, X₁₅ is Y, X₁₆ is V, X₁₇ is L, X₁₈ is I, X₁₉ is Y, X₂₀ is A, X₂₁ is S, X₂₂ is Q, X₂₃ is T, or a combination thereof (AB-6);
g) X₁ is T, X₂ is R, X₃ is S, X₄ is T, X₅ is T, X₆ is K, X₇ is R, X₈ is L, X₉ is T, X₁₀ is A, X₁₁ is S, X₁₂ is V, X₁₃ is R, X₁₄ is I, X₁₅ is V, X₁₆ is V, X₁₇ is L, X₁₈ is E, X₁₉ is Y, X₂₀ is A, X₂₁ is T, X₂₂ is K, X₂₃ is T, or a combination thereof (AB-7);
h) X₁ is T, X₂ is R, X₃ is S, X₄ is N, X₅ is T, X₆ is K, X₇ is R, X₈ is L, X₉ is T, X₁₀ is A, X₁₁ is S, X₁₂ is V, X₁₃ is R, X₁₄ is N, X₁₅ is Y, X₁₆ is V, X₁₇ is L, X₁₈ is E, X₁₉ is Y, X₂₀ is A, X₂₁ is S, X₂₂ is T, X₂₃ is T, or a combination thereof (AB-8);
i) X₁ is T, X₂ is R, X₃ is S, X₄ is N, X₅ is T, X₆ is K, X₇ is R, X₈ is L, X₉ is T, X₁₀ is T, X₁₁ is A, X₁₂ is V, X₁₃ is R, X₁₄ is S, X₁₅ is Y, X₁₆ is V, X₁₇ is L, X₁₈ is L, X₁₉ is Y, X₂₀ is A, X₂₁ is S, X₂₂ is Q, X₂₃ is T, or a combination thereof (AB-9);
j) X₁ is T, X₂ is R, X₃ is D, X₄ is K, X₅ is T, X₆ is R, X₇ is R, X₈ is L, X₉ is S, X₁₀ is S, X₁₁ is S, X₁₂ is A, X₁₃ is R, X₁₄ is V, X₁₅ is V, X₁₆ is V, X₁₇ is L, X₁₈ is V, X₁₉ is K, X₂₀ is A, X₂₁ is D, X₂₂ is F, X₂₃ is H, or a combination thereof (AB-10);
k) X₁ is T, X₂ is R, X₃ is T, X₄ is N, X₅ is T, X₆ is K, X₇ is R, X₈ is L, X₉ is S, X₁₀ is V, X₁₁ is S, X₁₂ is T, X₁₃ is R, X₁₄ is K, X₁₅ is Y, X₁₆ is V, X₁₇ is D, X₁₈ is Y, X₁₉ is Y, X₂₀ is A, X₂₁ is S, X₂₂ is H, X₂₃ is T, or a combination thereof (AB-11);
l) X₁ is T, X₂ is R, X₃ is S, X₄ is N, X₅ is K, X₆ is K, X₇ is R, X₈ is L, X₉ is T, X₁₀ is S, X₁₁ is R, X₁₂ is V, X₁₃ is R, X₁₄ is Y, X₁₅ is Y, X₁₆ is V, X₁₇ is L, X₁₈ is Q, X₁₉ is Y, X₂₀ is A, X₂₁ is S, X₂₂ is F, X₂₃ is T, or a combination thereof (AB-12);
m) X₁ is T, X₂ is R, X₃ is S, X₄ is N, X₅ is T, X₆ is K, X₇ is R, X₈ is W, X₉ is T, X₁₀ is S, X₁₁ is S, X₁₂ is V, X₁₃ is R, X₁₄ is V, X₁₅ is R, X₁₆ is I, X₁₇ is L, X₁₈ is H, X₁₉ is Y, X₂₀ is A, X₂₁ is S, X₂₂ is Y, X₂₃ is T, or a combination thereof (AB-13);
n) X₁ is T, X₂ is R, X₃ is S, X₄ is N, X₅ is I, X₆ is K, X₇ is R, X₈ is Y, X₉ is T, X₁₀ is K, X₁₁ is S, X₁₂ is V, X₁₃ is R, X₁₄ is V, X₁₅ is R, X₁₆ is I, X₁₇ is L, X₁₈ is H, X₁₉ is Y, X₂₀ is A, X₂₁ is S, X₂₂ is K, X₂₃ is T, or a combination thereof (AB-14c);
o) X₁ is N, X₂ is K, X₃ is S, X₄ is N, X₅ is T, X₆ is K, X₇ is R, X₈ is W, X₉ is T, X₁₀ is S, X₁₁ is S, X₁₂ is V, X₁₃ is K, X₁₄ is R, X₁₅ is R, X₁₆ is I, X₁₇ is L, X₁₈ is R, X₁₉ is Y, X₂₀ is A, X₂₁ is S, X₂₂ is Y, X₂₃ is T, or a combination thereof (AB-15c).

### Constant Domains

In some embodiments, a polypeptide comprises:
a) an antibody heavy chain constant domain sequence;
b) an antibody light chain constant domain sequence; or
both a) and b).

In some embodiments, a polypeptide comprises an antibody heavy chain constant domain sequence. In some embodiments, an antibody heavy chain constant domain is selected from the group consisting of an IgA constant domain, an IgD constant domain, an IgE constant domain, an IgG constant domain and an IgM constant domain.

In some embodiments, an IgG constant domain is an IgG1 constant domain, an IgG2 constant domain, an IgG3 constant domain or an IgG4 constant domain. In some embodiments, the IgG2 constant domain is an IgG2a, an IgG2b constant domain or an IgG2c constant domain. In some embodiments, an IgA constant domain is an IgA1 constant domain or an IgA2 constant domain. In some embodiments, the antibody heavy chain constant domain is an IgG1 constant domain (e.g., IGHV1-5 or IGHV5-51).

In some embodiments, the name of a polypeptide comprising a human IgG1 (hIgG1) domain is followed by “d”. In some embodiments, the name of a polypeptide comprising a human IgG4 (hIgG4) domain is followed by “c”. In some embodiments, the name of a polypeptide comprising a human IgG4 LS variant (hIgG4 LS) domain is followed by “b”. In some embodiments, the name of a polypeptide comprising a human IgG4 YTE variant (hIgG4 YTE) domain is followed by “a”.

In some embodiments, an antibody heavy chain constant domain sequence has at least about 60% sequence identity to the amino acid sequence of any one of SEQ ID NOs: 98-101. For example, an antibody heavy chain constant domain sequence can have at least about: 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of any one of SEQ ID NOs: 98-101. In some embodiments, the antibody heavy chain constant domain sequence has at least about 70% or at least about 80% sequence identity to the amino acid sequence of any one of SEQ ID NOs: 98-101.

In some embodiments, an antibody heavy chain constant domain sequence comprises at least one amino acid substitution relative to the amino acid sequence of any one of SEQ ID NOs: 98-101. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, an antibody heavy chain constant domain sequence comprises about 1-10 amino acid substitutions, relative to the amino acid sequence of any one of SEQ ID NOs: 98-101.

In some embodiments, a polypeptide comprises an Fc polypeptide, or Fc domain (e.g., an IgG4 domain). In certain embodiments, the Fc domain comprises a mutation that decreases (e.g., inhibits, ablates) an effector function of the Fc domain. See, e.g., Dumet et al., Insights into the IgG heavy chain engineering patent landscape as applied to IgG4 antibody development, MAbs. 11 (8): 1341-50 (2019) (particularly Tables 1 and 2 therein) and WO02060919, the contents of which are incorporated by reference herein in their entirety. In particular embodiments, the Fc domain comprises LS (M428L/N434S by Kabat numbering) or YTE (M252Y/S254T/T256E by Kabat numbering).

In some embodiments, a polypeptide comprises an antibody light chain constant domain sequence.

In some embodiments, an antibody light chain constant domain is selected from the group consisting of a k constant domain and a 2 constant domain.

In some embodiments, an antibody light chain constant domain sequence has at least about 60% sequence identity to the amino acid sequence of SEQ ID NO:102 or SEQ ID NO:103. For example, an antibody light chain constant domain sequence can have at least about: 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO: 102 or SEQ ID NO:103. In some embodiments, an antibody light chain constant domain sequence has at least about 70% or at least about 80% sequence identity to SEQ ID NO:102 or SEQ ID NO:103.

In some embodiments, an antibody light chain constant domain sequence comprises at least one amino acid substitution relative to the amino acid sequence of SEQ ID NO:102 or SEQ ID NO:103. For example, the number of amino acid substitutions can be at least about: 2, 3, 4, 5, 6, 7, 8, 9, 10, 11, 12, 13, 14, 15, 16, 17, 18, 19 or 20, or about: 1-20, 1-19, 2-19, 2-18, 2-17, 3-17, 3-16, 4-16, 4-15, 5-15, 5-14, 6-14, 6-13, 7-13, 7-12, 8-12, 8-11 or 9-11. In some embodiments, an antibody light chain constant domain sequence comprises about 1-10 amino acid substitutions, relative to the amino acid sequence of SEQ ID NO:102 or SEQ ID NO:103. In some embodiments, the at least one amino acid substitution is a conservative substitution. In some embodiments, the at least one amino acid substitution is a highly conservative substitution.

In some embodiments, a polypeptide comprises:
a) an antibody heavy chain constant domain sequence; and
b) an antibody light chain constant domain sequence.

In some embodiments, an antibody heavy chain constant domain is an IgG4 constant domain (e.g., SEQ ID NO:98), and an antibody light chain constant domain is a k constant domain (e.g., SEQ ID NO:102).

### Antibodies and Antigen Binding Fragments

In some embodiments, a polypeptide is an immunoglobulin molecule, such as an antibody (e.g., a whole antibody, an intact antibody) or an antigen-binding fragment of an antibody.

In some embodiments, a polypeptide is an antibody. As used herein, the term “antibody” refers to an immunoglobulin molecule capable of specific binding to a target, such as a carbohydrate, polynucleotide, lipid, polypeptide, etc., through at least one antigen recognition site, located in the variable domain of the immunoglobulin molecule. As used herein, the term “antibody” refers to a full-length antibody.

In some embodiments, a polypeptide is an antibody comprising two heavy (H) chains and two light (L) chains inter-connected by disulfide bonds or multimers thereof (for example, IgM). Each heavy chain comprises a V_{H} and a heavy chain constant domain (comprising domains CH1, hinge CH2 and CH3). Each light chain comprises a V_{L} and a light chain constant domain (CL). V_{H} and V_{L} regions may be further subdivided into regions of hypervariability, termed complementarity determining regions (CDRs), interspersed within framework regions (FRs). V_{H} and V_{L} each comprises three CDRs and four FRs, arranged from the amino-terminus to the carboxy-terminus in the following order: FR1, CDR1, FR2, CDR2, FR3, CDR3, and FR4. An antibody can be of any species, such as a murine antibody, a human antibody, or a humanized antibody.

In some embodiments, a polypeptide comprises a heavy chain amino acid sequence set forth in any one of SEQ ID NOs: 105-160. In some embodiments, a polypeptide comprises a light chain amino acid sequence set forth in any one of SEQ ID NOs: 162-176.

In some embodiments, a polypeptide comprises a heavy chain amino acid sequence set forth in any one of SEQ ID NOs: 105-117. In some embodiments, a polypeptide comprises a light chain amino acid sequence set forth in any one of SEQ ID NOs: 162-174. In some embodiments, a polypeptide comprises a heavy chain amino acid sequence set forth in any one of SEQ ID NOs: 105-117, and a light chain amino acid sequence set forth in any one of SEQ ID NOs: 162-174.

In some embodiments, a polypeptide comprises a heavy chain amino acid sequence set forth in any one of SEQ ID NOs: 131-145. In some embodiments, a polypeptide comprises a light chain amino acid sequence set forth in any one of SEQ ID NOs: 162-176. In some embodiments, a polypeptide comprises a heavy chain amino acid sequence set forth in any one of SEQ ID NOs: 131-145, and a light chain amino acid sequence set forth in any one of SEQ ID NOs: 162-176.

In some embodiments, a polypeptide comprises a heavy chain amino acid sequence set forth in any one of SEQ ID NOs: 148-160. In some embodiments, a polypeptide comprises a light chain amino acid sequence set forth in any one of SEQ ID NOs: 162-174. In some embodiments, a polypeptide comprises a heavy chain amino acid sequence set forth in any one of SEQ ID NOs: 148-160, and a light chain amino acid sequence set forth in any one of SEQ ID NOs: 162-174.

In some embodiments, a polypeptide comprises a heavy chain amino acid sequence set forth in any one of SEQ ID NOs: 118-130. In some embodiments, a polypeptide comprises a light chain amino acid sequence set forth in any one of SEQ ID NOs: 162-174. In some embodiments, a polypeptide comprises a heavy chain amino acid sequence set forth in any one of SEQ ID NOs: 118-130, and a light chain amino acid sequence set forth in any one of SEQ ID NOs: 162-174.

In some embodiments, a polypeptide comprises:
a) a heavy chain comprising the amino acid sequence of SEQ ID NO:118; and
b) a light chain comprising the amino acid sequence of SEQ ID NO:162 (AB-1b).

In some embodiments, a polypeptide comprises:
a) a heavy chain comprising the amino acid sequence of SEQ ID NO:119; and
b) a light chain comprising the amino acid sequence of SEQ ID NO:163 (AB-2b).

In some embodiments, a polypeptide comprises:
a) a heavy chain comprising the amino acid sequence of SEQ ID NO:120; and
b) a light chain comprising the amino acid sequence of SEQ ID NO:164 (AB-3b).

In some embodiments, a polypeptide comprises:
a) a heavy chain comprising the amino acid sequence of SEQ ID NO: 121; and
b) a light chain comprising the amino acid sequence of SEQ ID NO:165 (AB-4b).

In some embodiments, a polypeptide comprises:
a) a heavy chain comprising the amino acid sequence of SEQ ID NO:122; and
b) a light chain comprising the amino acid sequence of SEQ ID NO:166 (AB-4B).

In some embodiments, a polypeptide comprises:
a) a heavy chain comprising the amino acid sequence of SEQ ID NO:123; and
b) a light chain comprising the amino acid sequence of SEQ ID NO:167 (AB-6b).

In some embodiments, a polypeptide comprises:
a) a heavy chain comprising the amino acid sequence of SEQ ID NO:124; and
b) a light chain comprising the amino acid sequence of SEQ ID NO:168 (AB-7b).

In some embodiments, a polypeptide comprises:
a) a heavy chain comprising the amino acid sequence of SEQ ID NO:125; and
b) a light chain comprising the amino acid sequence of SEQ ID NO:169 (AB-8b).

In some embodiments, a polypeptide comprises:
a) a heavy chain comprising the amino acid sequence of SEQ ID NO:126; and
b) a light chain comprising the amino acid sequence of SEQ ID NO:170 (AB-9b).

In some embodiments, a polypeptide comprises:
a) a heavy chain comprising the amino acid sequence of SEQ ID NO:127; and
b) a light chain comprising the amino acid sequence of SEQ ID NO:171 (AB-10b).

In some embodiments, a polypeptide comprises:
a) a heavy chain comprising the amino acid sequence of SEQ ID NO:128; and
b) a light chain comprising the amino acid sequence of SEQ ID NO:172 (AB-11b).

In some embodiments, a polypeptide comprises:
a) a heavy chain comprising the amino acid sequence of SEQ ID NO:129; and
b) a light chain comprising the amino acid sequence of SEQ ID NO:173 (AB-12b).

In some embodiments, a polypeptide comprises:
a) a heavy chain comprising the amino acid sequence of SEQ ID NO:130; and
b) a light chain comprising the amino acid sequence of SEQ ID NO:174 (AB-13b).

In some embodiments, a polypeptide is a single-domain antibody or an antigen-binding fragment thereof. As used herein, the term “single-domain antibody (sdAb)” or “nanobody” refers to an immunoglobulin molecule consisting of a single monomeric variable antibody domain and capable of specific binding to a target. A single-domain antibody can be of any species, such as a murine antibody, a human antibody or a humanized single-domain antibody.

In some embodiments, a V_{H} domain and a V_{L} domain may be linked together via a linker (e.g., a synthetic linker) to form various types of single-chain antibody designs in which the V_{H}/V_{L} domains pair intramolecularly, or intermolecularly in those cases when the V_{H} and V_{L} domains are expressed by separate chains, to form a monovalent antigen binding site.

In some embodiments, a polypeptide is a heavy-chain antibody comprising two or more heavy chains, but lacking light chain, or an antigen-binding fragment thereof. Non-limiting examples of heavy chain antibodies include camelid Vhh (also referred to as VHH or V_{H}H) antibodies. Camelid antibodies are antibodies from the Camelidae family of mammals that include llamas, camels, and alpacas.

In some embodiments, a polypeptide is an antibody mimetic. The term “antibody mimetic” refers to polypeptides capable of mimicking an antibody's ability to bind an antigen, but structurally differ from native antibody structures. Non-limiting examples of antibody mimetics include Adnectins, Affibodies, Affilins, Affimers, Affitins, Alphabodies, Anticalins, Avimers, DARPins, Fynomers, Kunitz domain peptides, monobodies, nanobodies, nanoCLAMPs, and Versabodies.

In some embodiments, a polypeptide is an antigen-binding fragment of an antibody. The term “antigen-binding fragment” refers to a portion of an immunoglobulin molecule (e.g., antibody) that retains the antigen binding properties of the full-length antibody. Non-limiting examples of antigen-binding fragments include a V_{H} region, a V_{L} region, an Fab fragment, an F(ab′)₂ fragment, an Fd fragment, an Fv fragment, and a domain antibody (dAb) consisting of one V_{H} domain or one V_{L} domain, etc. V_{H} and V_{L} domains may be linked together via a synthetic linker to form various types of single-chain antibody designs in which the V_{H}/V_{L} domains pair intramolecularly, or intermolecularly in those cases when the V_{H} and V_{L} domains are expressed by separate chains, to form a monovalent antigen binding site, such as single chain Fv (scFv) or diabody. In some embodiments, a polypeptide disclosed herein is an antigen binding fragment selected from Fab, F(ab′)₂, Fab′, scFv, or Fv. In some embodiments, a polypeptide is a scFv.

In some embodiments, a polypeptide is an isolated polypeptide.

In some embodiments, a polypeptide (e.g., an isolated polypeptide) is recombinantly produced. In some embodiments, a polypeptide (e.g., an isolated polypeptide) is synthetically produced.

In some embodiments, a polypeptide is linked to a second polypeptide. The term “linked” means attached, via a covalent or noncovalent interaction. Conjugation can employ a suitable linking agent. Non-limiting examples include peptide linkers, compound linkers, and chemical cross-linking agents. In some embodiments, the linker is a disulfide bond.

In some embodiments, a polypeptide is conjugated to a heterologous moiety. The term “conjugated” refers to attached, via a covalent or noncovalent interaction. Conjugation can employ any one or more of suitable linking agents. Non-limiting examples include peptide linkers, compound linkers, and chemical cross-linking agents.

In some embodiments, a heterologous moiety comprises a therapeutic agent, a diagnostic agent, or both. In some embodiments, a heterologous moiety is selected from polyethylene glycol (PEG), hexadecanoic acid, hydrogels, nanoparticles, multimerization domains and carrier peptides.

In some embodiments, a nanoparticle is a lipid nanoparticle. In some embodiments, a nanoparticle is a polymer nanoparticle. In some embodiments, a polymer is an amphiphilic polymer. In some embodiments, a polymer is a hydrophobic or hydrophilic polymer. Non-limiting examples of polymers include poly(lactic acid)-poly(ethylene glycol), poly(lactic-co-glycolic acid)-poly(ethylene glycol), poly(lactic-co-glycolic) acid (PLGA), poly(lactic-co-glycolic acid)-d-α-tocopheryl polyethylene glycol succinate, poly(lactic-co-glycolic acid)-ethylene oxide fumarate, poly(glycolic acid)-poly(ethylene glycol), polycaprolactone-poly(ethylene glycol), or any salts thereof. In some embodiments, a polymer nanoparticle comprises poly(lactic-co-glycolic) acid (PLGA).

In some embodiments, a carrier polypeptide is albumin or an Fc polypeptide.

In some embodiments, a polypeptide binds an IL-4Rα with a binding constant (K_{D}) of about 1 μM or less. As used herein the term “K_{D},” also referred to as “binding constant,” “equilibrium dissociation constant” or “affinity constant,” is a measure of the extent of a reversible association between two molecular species (e.g., antibody and target protein) and includes both the actual binding affinity as well as the apparent binding affinity. Binding affinity can be determined using methods known in the art including, for example, by measurement of surface plasmon resonance, e.g., using a Biolayer interferometry (Octet, ForteBio) or a surface plasmon resonance (Biacore) system and assay. A reference that compares various surface technologies for measuring binding affinity and kinetics is Yang, D., Singh, A., Wu, H., & Kroe-Barrett, R., Comparison of biosensor platforms in the evaluation of high affinity antibody-antigen binding kinetics, Analytical Biochemistry 508:78-96 (2016), the contents of which are incorporated by reference herein in their entirety.

In some embodiments, a polypeptide binds an IL-4Rα with a binding constant (K_{D}) of about 0.05 to 0.5 nM or less or about 0.02 to 0.04 nM or less.

In some embodiments, a polypeptide binds an IL-4Rα with an association constant (ka) of about 100×10⁵ M⁻¹ s⁻¹ or less, about 7×10⁵ to 9×10⁵ M⁻¹ s⁻¹ or less, or about 7.7×10⁵ to 8.9×10⁵ M⁻¹ s⁻¹ or less.

In some embodiments, a polypeptide dissociates from an IL-4Rα with a dissociation constant (k_{d}) of about 100×10⁻⁵ s⁻¹ or less, 2×10⁻⁵ to 3×10⁻⁵ s⁻¹ or less, or about 2.1×10⁻⁵ to 2.6×10⁻⁵ s⁻¹ or less.

In some embodiments, a polypeptide binds an IL-4Rα with an EC₅₀ of about 1 μM or less, about 0.02 to 1.5 nM or less, or about 0.04 to 1.3 nM or less.

In some embodiments, a polypeptide blocks IL-4 Type II signaling with an IC₅₀ of about 1 μM or less, about 1.1 to 7.3 nM or less, about 4.4 to 7.3 nM or less, or about 1.1 to 1.8 nM or less.

In some embodiments, a polypeptide blocks IL-13 Type II signaling with an IC₅₀ of about 1 μM or less or about 3 to 3.3 nM or less.

In some embodiments, a polypeptide inhibits CD23 expression in B cells with an IC₅₀ of about 1 μM or less, about 8 to 50 nM or less, about 30 to 50 nM or less, about 30 to 34 nM or less, or about 8 to 8.8 nM or less.

In some embodiments, a polypeptide competes with the Reference Antibody for binding to an interleukin-4 receptor alpha (IL-4Rα). Techniques and assays for assessing competition between antibodies are known in the art.

In some embodiments, a polypeptide has a weaker self-association than the Reference Antibody, for example, as determined by an affinity-capture self-interaction nanoparticle spectroscopy (AC-SINS) value. The AC-SINS value is the change in maximum absorbance wavelength in the coated-nanoparticle absorption spectra compared to the spectra of the nanoparticle alone. Thus, the greater the change in maximum absorbance wavelength, the more self-interaction of the antibody coated on the nanoparticle. Self-association is an unwanted property that correlates with poor viscosity and poor PK properties. Techniques and assays for assessing self-association of proteins are known in the art. See, e.g., Patro & Przybycien, Biotechnol Bioeng. 52 (2): 193-203 (1996), the contents of which are incorporated herein in their entirety. In some embodiments, a polypeptide has a weaker self-association than the Reference Antibody.

In some embodiments, a polypeptide has an AC-SINS value of no more than about: 1, 2, 3, 4, 5, 6, 7, 8, 9, 10, 11, 12, 13, 14, 15, 16, 17, 18, 18, 20, 21, 22, 23, 24 or 25. In some embodiments, a polypeptide has an AC-SINS value of no more than about 14. In some embodiments, a polypeptide has an AC-SINS value of no more than about 8. In some embodiments, a polypeptide has an AC-SINS value of about: 1, 2, 3, 4, 5, 6, 7, 8, 9, 10, 11, 12, 13, 14, 15, 16, 17, 18, 18, 20, 21, 22, 23, 24 or 25. In some embodiments, a polypeptide has an AC-SINS value of about 0-25, e.g., 0-20, 0-15, 0-10, 0-8, 0-5, 2-20, 2-15, 2-10, 2-8, 2-5, 5-20, 5-15, 5-10, 5-8, 7-8 or 13-15. In some embodiments, a polypeptide has an AC-SINS value of about 13-14, 13-15, 7-9 or 7-8. In some embodiments, a polypeptide has an AC-SINS value of about: 8 or 14.

In some embodiments, a polypeptide has an improved developability (e.g., reduced AC-SINS) relative to the Reference Antibody. In some embodiments, the self-association of a polypeptide is at least about 10% lower than that of the Reference Antibody, for example, by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99% lower than that of the Reference Antibody. In some embodiments, the self-association of a polypeptide is at least about 30% lower than that of the Reference Antibody.

In some embodiments, the self-association of a polypeptide is less than about 90% of that of the Reference Antibody, for example, less than about: 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2% or 1% of that of the Reference Antibody.

In some embodiments, the self-association of a polypeptide is about 1-90% relative to that of the Reference Antibody, for example, about: 2-90%, 2-85%, 3-85%, 3-80%, 4-80%, 4-75%, 5-75%, 5-70%, 6-70%, 6-65%, 7-65%, 7-60%, 8-60%, 8-55%, 9-55%, 9-50%, 10-50%, 10-45%, 15-45%, 15-40%, 20-40%, 20-35%, 25-35% or 25-30%, relative to that of the Reference Antibody.

In some embodiments, the reduction in self-association relative to the Reference Antibody is at least about 10%, for example, by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%.

### Fusion Proteins

In some embodiments, the disclosure provides a fusion protein comprising one or more of polypeptides described herein.

The term “fusion protein” refers to a synthetic, semi-synthetic or recombinant single protein molecule. A fusion protein can comprise all or a portion of two or more different proteins and/or polypeptides that are attached by covalent bonds (e.g., peptide bonds). For example, a fusion protein can comprise a full-length polypeptide disclosed herein (e.g., a whole antibody), or a fragment thereof (e.g., an antigen-binding fragment of an antibody). The heterologous partner can be a full-length protein or a fragment thereof (e.g., a truncated protein).

Fusion proteins can be produced recombinantly or synthetically, using routine methods and reagents that are well known in the art. For example, a fusion protein disclosed herein can be produced recombinantly in a suitable host cell (e.g., bacteria) according to methods known in the art. See, e.g., Current Protocols in Molecular Biology, Second Edition, Ausubel et al. eds., John Wiley & Sons, 1992; and Molecular Cloning: a Laboratory Manual, 2nd edition, Sambrook et al., 1989, Cold Spring Harbor Laboratory Press. For example, a nucleic acid molecule comprising a nucleotide sequence encoding a fusion protein described herein can be introduced and expressed in suitable host cell (e.g., E. coli), and the expressed fusion protein can be isolated/purified from the host cell (e.g., in inclusion bodies) using routine methods and readily available reagents. For example, DNA fragments coding for different protein sequences (e.g., a light-responsive domain, a heterologous peptide component) can be ligated together in-frame in accordance with conventional techniques. In another embodiment, the fusion gene can be synthesized by conventional techniques including automated DNA synthesizers. In some embodiments, PCR amplification of nucleic acid fragments can be carried out using anchor primers that give rise to complementary overhangs between two consecutive nucleic acid fragments that can subsequently be annealed and re-amplified to generate a chimeric nucleic acid sequence (see Ausubel et al., Current Protocols in Molecular Biology, 1992).

### Nucleic Acids, Vectors, Host Cells

In some embodiments, the disclosure provides one or more polynucleotides (e.g., DNA, RNA, or analogs of either, e.g., optionally including one or more modified nucleotides; the polynucleotide may be linear or circular, e.g., linear or circular RNA) encoding any one of polypeptides or fusion proteins described herein. In some embodiments, a polypeptide or fusion protein disclosed herein is encoded by a single polynucleotide. In some embodiments, a polypeptide or fusion protein disclosed herein is encoded by multiple polynucleotides.

In some embodiments, the polynucleotide comprises a nucleotide sequence that is codon-optimized for a chosen host cell.

In some embodiments, the disclosure provides a vector (e.g., an expression vector, including a viral-delivery vector) comprising any one or more of the polynucleotides described herein.

The term “expression vector” refers to a replicable nucleic acid from which one or more proteins can be expressed when the expression vector is transformed into a suitable expression host cell.

In some embodiments, the vector (e.g., expression vector) comprises an expression control polynucleotide sequence operably linked to the polynucleotide, a polynucleotide sequence encoding a selectable marker, or both. In some embodiments, the expression control polynucleotide sequence comprises a promoter sequence, an enhancer sequence, or both. In some embodiments, the expression control polynucleotide sequence comprises an inducible promoter sequence. The term “promoter” refers to a region of DNA to which RNA polymerase binds and initiates the transcription of a gene. The term “operably linked” means that the nucleic acid is positioned in the recombinant polynucleotide, e.g., vector, in such a way that enables expression of the nucleic acid under control of the element (e.g., promoter) to which it is linked. The term “selectable marker element” is an element that confers a trait suitable for artificial selection. Selectable marker elements can be negative or positive selection markers.

In some embodiments, the disclosure provides an expression host cell comprising any one or more of the polynucleotides or expression vectors described herein.

The term “expression host cell” refers to a cell useful for receiving, maintaining, reproducing and/or amplifying a vector.

Non-limiting examples of expression host cells include mammalian cells such as hybridoma cells, Chinese hamster ovary (CHO) cells, COS cells, human embryonic kidney (HEK), yeast cells such as Pichia pastoris cells, or bacterial cells such as E. coli, including DH5α, etc.

### Compositions

In some embodiments, the disclosure provides a composition comprising any one of polypeptides or fusion proteins described herein. In some embodiments, the composition is a pharmaceutical composition.

In some embodiments, the composition (e.g., pharmaceutical composition) comprises pharmaceutically acceptable carriers, excipients, stabilizers, diluents or tonifiers (Remington's Pharmaceutical Sciences 16th edition, Osol, A. Ed. (1980)). Suitable pharmaceutically acceptable carriers, excipients, or stabilizers are nontoxic to recipients at the dosages and concentrations employed. Non-limiting examples of pharmaceutically acceptable carriers, excipients, stabilizers, diluents or tonifiers include buffers (e.g., phosphate, citrate, histidine), antioxidants (e.g., ascorbic acid or methionine), preservatives, proteins (e.g., serum albumin, gelatin or immunoglobulins); hydrophilic polymers, amino acids, carbohydrates (e.g., monosaccharides, disaccharides, glucose, mannose or dextrins); chelating agents (e.g., EDTA), sugars (e.g., sucrose, mannitol, trehalose or sorbitol), salt-forming counter-ions (e.g., sodium), metal complexes (e.g., Zn-protein complexes); non-ionic surfactants (e.g., Tween), PLURONICS™ and polyethylene glycol (PEG).

In some embodiments, the composition (e.g., pharmaceutical composition) disclosed herein is formulated for a suitable administration schedule and route. Non-limiting examples of administration routes include oral, rectal, mucosal, intravenous, intramuscular, subcutaneous and topical, etc. In some embodiments, the composition (e.g., pharmaceutical composition) disclosed herein is stored in the form of an aqueous solution or a dried formulation (e.g., lyophilized).

In some embodiments, the composition is formulated to be administered by infusion (e.g., intravenous infusion).

In some embodiments, the composition is formulated to be administered with a second therapeutic agent as a combination therapy. In some embodiments, the second therapeutic agent is any one of polypeptides described herein. In some embodiments, the second therapeutic agent comprises an agent indicated for treatment of an inflammatory condition, non-limiting examples of which include: atopic dermatitis, asthma, eczema (atopic dermatitis), food allergy, prurigo nodularis, chronic rhinosinusitis with nasal polyps, keloids, eosinophilic esophagitis, prostate cancer, chronic urticaria, bullous pemphigoid, localized scleroderma, alopecia areata, ulcerative colitis, aspirin-exacerbated respiratory disease, metastatic non-small cell lung cancer, Netherton syndrome, and combinations thereof.

In some embodiments, non-limiting examples of the second therapeutic agent include: an additional anti-IL-4Rα antibody or antigen-binding fragment thereof, an inhaled corticosteroid, a leukotriene modifier, a combination corticosteroid/long-acting beta agonist inhaler, a bronchodilator, a short-acting beta agonist, an anticholinergic agent, an oral or intravenous corticosteroid, a calcineurin inhibitor, an anti-inflammatory agent, an anti-rheumatic agent, an immunosuppressant, a biologic agent, an antihistamine, an epinephrine, an oral immunotherapy, a topical corticosteroid, a topical anesthetic, an immunomodulatory agent, a nasal corticosteroid, an antifungal agent, an anti-androgen agent, a luteinizing hormone-releasing hormone (LHRH) agonist, an gonadotropin-releasing hormone (GnRH) antagonist, a chemotherapy, a targeted drug therapy, a steroid-sparing agent, and combinations thereof.

### Methods of Use

In some embodiments, the disclosure provides methods of treating a subject in need thereof, comprising administering an effective amount of the composition disclosed herein. In some embodiments, the composition comprises a pharmaceutically acceptable carrier and, wherein as an active ingredient, any one of polypeptides or fusion proteins described herein.

In some embodiments, the amount of pathogenic IL-4/IL-13 signaling in the subject is reduced by at least about 10%, e.g., by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%.

In some embodiments, the likelihood of pathogenic IL-4/IL-13 signaling in the subject in the presence of a polypeptide is about 1-90% relative to the likelihood in the absence of the polypeptide, for example, about: 2-90%, 2-85%, 3-85%, 3-80%, 4-80%, 4-75%, 5-75%, 5-70%, 6-70%, 6-65%, 7-65%, 7-60%, 8-60%, 8-55%, 9-55%, 9-50%, 10-50%, 10-45%, 15-45%, 15-40%, 20-40%, 20-35%, 25-35% or 25-30%.

The term “subject” and “patient” are used herein interchangeably to refer to an animal (e.g., a mammal, such as a human) who is to be treated according to a method disclosed herein. A subject to be treated according to methods described herein may be one who has been diagnosed with a particular condition (e.g., in inflammatory condition), or one at risk of developing such conditions or symptoms of such conditions. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

In some embodiments, the subject has (e.g., confirmed by testing), or is suspected of having, an inflammatory condition. In some embodiments, the subject has an inflammatory condition. In some embodiments, the subject has been diagnosed with an inflammatory condition. In some embodiments, the subject is at risk of developing an inflammatory condition.

In some embodiments, the subject is a mammal. In some embodiments, the subject is a mammal selected from the group consisting of a dog, a cat, a mouse, a rat, a hamster, a guinea pig, a horse, a pig, a sheep, a cow, a chimpanzee, a macaque, a cynomolgus, and a human. In some embodiments, the subject is a primate. In some embodiments, the subject is a human.

In some embodiments, the subject has asthma, eczema (atopic dermatitis), food allergy, prurigo nodularis, chronic rhinosinusitis with nasal polyps, keloids, eosinophilic esophagitis, prostate cancer, chronic urticaria, bullous pemphigoid, localized scleroderma, alopecia areata, ulcerative colitis, aspirin-exacerbated respiratory disease, metastatic non-small cell lung cancer, Netherton syndrome, or a combination thereof.

In some embodiments, the subject is immune-compromised (e.g., has an underlying disorder or is on immunosuppressive therapy).

In some embodiments, the subject is 40 years or older, e.g., at least: 45, 50, 55, 60, 65, 70, 75, 80, 85, 90 years old.

“A therapeutically effective amount,” “an effective amount” or “an effective dosage” is an amount effective, at dosages and for periods of time necessary, to achieve a desired therapeutic result (e.g., treatment, healing, inhibition or amelioration of physiological response or condition, etc.). The therapeutic effect does not necessarily occur by administration of one dose, and may occur only after administration of a series of doses. Thus, a therapeutically effective amount may be administered in one or more administrations. A therapeutically effective amount may vary according to factors such as disease state, age, sex, and weight of a mammal, mode of administration and the ability of a therapeutic, or combination of therapeutics, to elicit a desired response in an individual.

An effective amount of an agent to be administered can be determined by a clinician of ordinary skill using the guidance provided herein and other methods known in the art. Relevant factors include the given agent, the pharmaceutical formulation, the route of administration, the type of disease or disorder, the identity of the subject (e.g., age, sex, weight) or host being treated, and the like. For example, suitable dosages can be from about 0.001 mg/kg to about 100 mg/kg, from about 0.01 mg/kg to about 100 mg/kg, from about 0.01 mg/kg to about 10 mg/kg, from about 0.01 mg/kg to about 1 mg/kg body weight per treatment. Determining the dosage for a particular agent, subject and disease is well within the abilities of one of skill in the art. Preferably, the dosage does not cause or produces minimal adverse side effects.

Desired response or desired results include effects at the cellular level, tissue level, or clinical results. As such, “a therapeutically effective amount” or synonym thereto depends upon the context in which it is being applied. For example, in some embodiments it is an amount of the composition sufficient to achieve a treatment response as compared to the response obtained without administration of the composition. In some embodiments, it is an amount that results in a beneficial or desired result in a subject as compared to a control. As defined herein, a therapeutically effective amount of a composition disclosed herein may be readily determined by one of ordinary skill by routine methods known in the art. Dosage regimen and route of administration may be adjusted to provide the optimum therapeutic response.

In some embodiments, methods disclosed herein are used for prophylactic therapy. In some embodiments, the effective dosage is sufficient to prevent the subject from experiencing symptoms of an inflammatory condition.

In some embodiments, methods disclosed herein are used for treating an inflammatory condition (e.g., atopic dermatitis).

The term “treating” or “treatment” refers to the medical management of a subject with the intent to improve, ameliorate, stabilize (i.e., not worsen), prevent or cure a disease, pathological condition, or disorder-such as the particular indications exemplified herein. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition; preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

In some embodiments, the effective dosage is sufficient to reduce IL-4/IL-13 signaling in the subject. In some embodiments, the reduction in IL-4/IL-13 signaling is by at least about 10%, e.g., by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%. In some embodiments, the reduction in viral load is about 10-99%, e.g., about: 10-98%, 15-98%, 15-97%, 20-97%, 20-96%, 25-96%, 25-95%, 30-95%, 30-94%, 35-94%, 35-93%, 40-93%, 40-92%, 45-92%, 45-91%, 50-91%, 50-90%, 55-90%, 55-85%, 60-85%, 60-80%, 65-80%, 65-75%, or 70-75%.

In some embodiments, the effective dosage is sufficient to inhibit binding of IL-4 or IL-13 to IL-4Rα, target cells, or both. In some embodiments, the reduction in binding is by at least about 10%, e.g., by at least about: 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or 99%. In some embodiments, the reduction in binding is about 10-99%, e.g., about: 10-98%, 15-98%, 15-97%, 20-97%, 20-96%, 25-96%, 25-95%, 30-95%, 30-94%, 35-94%, 35-93%, 40-93%, 40-92%, 45-92%, 45-91%, 50-91%, 50-90%, 55-90%, 55-85%, 60-85%, 60-80%, 65-80%, 65-75%, or 70-75%.

A therapeutic agent described herein can be administered via a variety of routes of administration, including, for example, oral, dietary, topical, transdermal, rectal, parenteral (e.g., intra-arterial, intravenous, intramuscular, subcutaneous injection, intradermal injection), intravenous infusion and inhalation (e.g., intrabronchial, intranasal or oral inhalation, intranasal drops) routes of administration, depending on the compound and the particular disease to be treated. Administration can be local or systemic as indicated. The preferred mode of administration can vary depending on the particular compound chosen.

In some embodiments, a polypeptide, composition, or pharmaceutical composition disclosed herein is administered to a subject as a monotherapy.

In some embodiments, a polypeptide, composition, or pharmaceutical composition disclosed herein is administered to a subject in combination with one or more additional therapeutic agents (e.g., concurrently or sequentially with one or more additional therapeutic agents) or prophylactic agents (e.g., concurrently or sequentially with one or more prophylactic agents). In some embodiments, a subject has been previously treated with one or more therapeutic agents prior to being administered a polypeptide, composition, or pharmaceutical composition disclosed herein. In some embodiments, methods disclosed herein comprise administering a therapeutically effective amount of one or more additional therapeutic agents to the subject at the same time as, or following administration of a polypeptide, composition, or pharmaceutical composition disclosed herein. In some embodiments, methods disclosed herein comprise administering a therapeutically effective amount of one or more prophylactic agents to the subject before, at the same time as, or following administration of a polypeptide, composition, or pharmaceutical composition disclosed herein. In some embodiments, the subject previously received a therapeutic or prophylactic agent.

Non-limiting examples of additional therapeutic agents include an additional anti-IL-4Rα antibody or antigen-binding fragment thereof, an inhaled corticosteroid, a leukotriene modifier, a combination corticosteroid/long-acting beta agonist inhaler, a bronchodilator, a short-acting beta agonist, an anticholinergic agent, an oral or intravenous corticosteroid, a calcineurin inhibitor, an anti-inflammatory agent, an anti-rheumatic agent, an immunosuppressant, a biologic agent, an antihistamine, an epinephrine, an oral immunotherapy, a topical corticosteroid, a topical anesthetic, an immunomodulatory agent, a nasal corticosteroid, an antifungal agent, an anti-androgen agent, a luteinizing hormone-releasing hormone (LHRH) agonist, an gonadotropin-releasing hormone (GnRH) antagonist, a chemotherapy, a targeted drug therapy, a steroid-sparing agent, and combinations thereof.

Administration of the two or more therapeutic agents encompasses co-administration of the therapeutic agents in a substantially simultaneous manner, such as in a pharmaceutical combination. In some embodiments, such administration encompasses co-administration in multiple containers, or separate containers (e.g., capsules, powders, and liquids) for each therapeutic agent. Such administration also encompasses use of each type of therapeutic agent in a sequential manner, either at approximately the same time or at different times. The composition described herein and the second therapeutic agent can be administered via the same administration route or via different administration routes.

In some embodiments, the disclosure provides methods of preventing one or more symptoms of an inflammatory condition in a subject, comprising administering to the subject an effective amount of a pharmaceutical composition comprising a pharmaceutically acceptable carrier and, wherein as an active ingredient, any one of polypeptides or fusion proteins described herein.

In some embodiments, the disclosure provides methods of treating an inflammatory condition in a subject, comprising administering to the subject an effective amount of a pharmaceutical composition comprising a pharmaceutically acceptable carrier and, wherein as an active ingredient, any polypeptide or fusion protein described herein.

In some embodiments, the disclosure provides methods of reducing one or more symptoms of an inflammatory condition in a subject, comprising administering to the subject an effective amount of a pharmaceutical composition comprising a pharmaceutically acceptable carrier and, wherein as an active ingredient, any polypeptide or fusion protein described herein.

In some embodiments, the disclosure provides methods of inhibiting binding of IL-4 or IL-13 to a target cell, comprising contacting the target cell an effective amount of any polypeptide or fusion protein described herein.

In some embodiments, the disclosure provides methods of inhibiting binding of IL-4 or IL-13 to a target protein on a target cell, comprising contacting the target cell an effective amount of any polypeptide or fusion protein described herein.

Headings used in this application are for convenience only and do not affect the interpretation of this application.

Preferred features of each of the aspects or embodiments provided by the invention are applicable to all of the other aspects or embodiments of the invention mutatis mutandis and, without limitation, are exemplified by the dependent claims and also encompass combinations and permutations of individual features (e.g., elements, including numerical ranges and exemplary embodiments) of particular embodiments and aspects of the invention, including the working examples. For example, particular experimental parameters exemplified in the working examples can be adapted for use in the claimed invention piecemeal without departing from the invention. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the invention, as recognized by the person having ordinary skill in the art following the teachings of the specification, can be claimed in any combination or permutation to the extent that they are novel and non-obvious over the prior art-thus, to the extent an element is described in one or more references known to the person having ordinary skill in the art, they may be excluded from the claimed invention by, inter alia, a negative proviso or disclaimer of the feature or combination of features.

TABLE 1
Non-limiting examples of
IL-4Rα sequences

| SEQ ID | Amino Acid Sequence |
|---|---|
| NO: 1 | MKVLQEPTCVSDYMSISTCEWKMNG |
| | PTNCSTELRLLYQLVFLLSEAHTCI |
| | PENNGGAGCVCHLLMDDVVSADNYT |
| | LDLWAGQQLLWKGSFKPSEHVKPRA |
| | PGNLTVHDTLLLTWSNPYPPDNYLY |
| | NHLTYAVNIPADFRIYNVTYLEPSL |
| | RIAARARVRAWAQLYNTTWSEWSPS |
| | TKW |
| NO: 2 | MGWLCSGLLFPVSCLVLLQVASSGN |
| | MKVLQEPTCVSDYMSISTCEWKMNG |
| | PTNCSTELRLLYQLVFLLSEAHTCI |
| | PENNGGAGCVCHLLMDDVVSADNYT |
| | LDLWAGQQLLWKGSFKPSEHVKPRA |
| | PGNLTVHTNVSDTLLLTWSNPYPPD |
| | NYLYNHLTYAVNIWSENDPADFRIY |
| | NVTYLEPSLRIAASTLKSGISYRAR |
| | VRAWAQCYNTTWSEWSPSTKWHNSY |
| | REPFEQHLLLGVSVSCIVILAVCLL |
| | CYVSITKIKKEWWDQIPNPARSRLV |
| | AIIIQDAQTLEELSYQALALFSGAQ |
| | HEKG |
| NO: 3 | MGWLCSGLLFPVSCLVLLQVASSGS |
| | MKVLQEPTCVSDYMSISTCEWKMGG |
| | PTNCSAELRLLYQLVFQSSETHTCV |
| | PENNGGVGCVCHLLMDDVVSMDNYT |
| | LDLWAGQQLLWKGSFKPSEHVKPRA |
| | PGNLTVHTNVSDTVLLTWSNPYPPD |
| | NYLYNDLTYAVNIWSENDPAYSRIH |
| | NVTYLKPTLRIPASTLKSGISYRAR |
| | VRAWAQHYNTTWSEWSPSTKWYNSY |
| | REPFEQ |

TABLE 2
Variable Domain Amion Acid Sequences

| | SEQ | |
|---|---|---|
| Name | ID | V_{H} Amino Acid Sequence |
| Consensus | NO: 4 | EVQLVESGGGLEQPGGSLRLSCAGSGFX₁FX₂DYAMTWVRQAPGKGLEWVSSISGSGX₃X₄X₅YY |
| | | ADSVKGRFTISRDNSKNTLYLQMNSLRAEDTAVYYCAX₆DX₇X₈X₉X₁₀X₁₁X₁₂X₁₃PX₁₄YX₁₅GLD |
| | | X₁₆WGQGTTVTVSS |
| Reference | NO: 5 | EVQLVESGGGLEQPGGSLRLSCAGSGFTFRDYAMTWVRQAPGKGLEWVSSISGSGGNTYYADS |
| | | VKGRFTISRDNSKNTLYLQMNSLRAEDTAVYYCAKDRLSITIRPRYYGLDVWGQGTTVTVSS |
| AB-1 | NO: 6 | EVQLVESGGGLEQPGGSLRLSCAGSGFTFSDYAMTWVRQAPGKGLEWVSSISGSGGNTYYADS |
| | | VKGRFTISRDNSKNTLYLQMNSLRAEDTAVYYCAKDALTIAIRPYYYGLDVWGQGTTVTVSS |
| AB-2 | NO: 7 | EVQLVESGGGLEQPGGSLRLSCAGSGFTFRDYAMTWVRQAPGKGLEWVSSISGSGSNTYYADS |
| | | VKGRFTISRDNSKNTLYLQMNSLRAEDTAVYYCARDRLTAAVRPYYYGLDVWGQGTTVTVSS |
| AB-3 | NO: 8 | EVQLVESGGGLEQPGGSLRLSCAGSGFTFRDYAMTWVRQAPGKGLEWVSSISGSGGNTYYADS |
| | | VKGRFTISRDNSKNTLYLQMNSLRAEDTAVYYCAKDRLSKTIRPYYYGLDVWGQGTTVTVSS |
| AB-4 | NO: 9 | EVQLVESGGGLEQPGGSLRLSCAGSGFTFSDYAMTWVRQAPGKGLEWVSSISGSGGNTYYADS |
| | | VKGRFTISRDNSKNTLYLQMNSLRAEDTAVYYCARDRLTITIRPRYYGLDVWGQGTTVTVSS |
| AB-5 | NO: 10 | EVQLVESGGGLEQPGGSLRLSCAGSGFTFSDYAMTWVRQAPGKGLEWVSSISGSGSNTYYADS |
| | | VKGRFTISRDNSKNTLYLQMNSLRAEDTAVYYCAKDRLTIAIRPYYYGLDVWGQGTTVTVSS |
| AB-6 | NO: 11 | EVQLVESGGGLEQPGGSLRLSCAGSGFTFRDYAMTWVRQAPGKGLEWVSSISGSGSNTYYADS |
| | | VKGRFTISRDNSKNTLYLQMNSLRAEDTAVYYCAKDRLTTKVRPEYYGLDVWGQGTTVTVSS |
| AB-7 | NO: 12 | EVQLVESGGGLEQPGGSLRLSCAGSGFTFRDYAMTWVRQAPGKGLEWVSSISGSGSTTYYADS |
| | | VKGRFTISRDNSKNTLYLQMNSLRAEDTAVYYCAKDRLTASVRPIYVGLDVWGQGTTVTVSS |
| AB-8 | NO: 13 | EVQLVESGGGLEQPGGSLRLSCAGSGFTFRDYAMTWVRQAPGKGLEWVSSISGSGSNTYYADS |
| | | VKGRFTISRDNSKNTLYLQMNSLRAEDTAVYYCAKDRLTASVRPNYYGLDVWGQGTTVTVSS |
| AB-9 | NO: 14 | EVQLVESGGGLEQPGGSLRLSCAGSGFTFRDYAMTWVRQAPGKGLEWVSSISGSGSNTYYADS |
| | | VKGRFTISRDNSKNTLYLQMNSLRAEDTAVYYCAKDRLTTAVRPSYYGLDVWGQGTTVTVSS |
| AB-10 | NO: 15 | EVQLVESGGGLEQPGGSLRLSCAGSGFTFRDYAMTWVRQAPGKGLEWVSSISGSGDKTYYADS |
| | | VKGRFTISRDNSKNTLYLQMNSLRAEDTAVYYCARDRLSSSARPVYVGLDVWGQGTTVTVSS |
| AB-11 | NO: 16 | EVQLVESGGGLEQPGGSLRLSCAGSGFTFRDYAMTWVRQAPGKGLEWVSSISGSGTNTYYADS |
| | | VKGRFTISRDNSKNTLYLQMNSLRAEDTAVYYCAKDRLSVSTRPKYYGLDVWGQGTTVTVSS |
| AB-12 | NO: 17 | EVQLVESGGGLEQPGGSLRLSCAGSGFTFRDYAMTWVRQAPGKGLEWVSSISGSGSNKYYADS |
| | | VKGRFTISRDNSKNTLYLQMNSLRAEDTAVYYCAKDRLTSRVRPYYYGLDVWGQGTTVTVSS |
| AB-13 | NO: 18 | EVQLVESGGGLEQPGGSLRLSCAGSGFTFRDYAMTWVRQAPGKGLEWVSSISGSGSNTYYADS |
| | | VKGRFTISRDNSKNTLYLQMNSLRAEDTAVYYCAKDRWTSSVRPVYRGLDIWGQGTTVTVSS |
| AB-14 | NO: 19 | EVQLVESGGGLEQPGGSLRLSCAGSGFTFRDYAMTWVRQAPGKGLEWVSSISGSGSNIYYADS |
| | | VKGRFTISRDNSKNTLYLQMNSLRAEDTAVYYCAKDRYTKSVRPVYRGLDIWGQGTTVTVSS |
| AB-15 | NO: 20 | EVQLVESGGGLEQPGGSLRLSCAGSGENFKDYAMTWVRQAPGKGLEWVSSISGSGSNTYYADS |
| | | VKGRFTISRDNSKNTLYLQMNSLRAEDTAVYYCAKDRWTSSVKPRYRGLDIWGQGTTVTVSS |
| AB-16 | NO: 21 | EVQLVESGGGLEQPGGSLRLSCAGSGFTFSDYAMTWVRQAPGKGLEWVSSISGSGGNTYYADS |
| | | VKGRFTISRDNSKNTLYLQMNSLRAEDTAVYYCAKDALSITIRPRYYGLDVWGQGTTVTVSS |
| AB-17 | NO: 22 | EVQLVESGGGLEQPGGSLRLSCAGSGFTERDYAMTWVRQAPGKGLEWVSSISGSGSNTYYADS |
| | | VKGRFTISRDNSKNTLYLQMNSLRAEDTAVYYCAKDRLTAAVRPRYYGLDVWGQGTTVTVSS |
| | SEQ | |
| Name | ID | V_{L} Amino Acid Sequence |
| Consensus | NO: 23 | DIVMTQSPLSLPVTPGEPASISCRSSQSLX₁₇YSX₁₈GX₁₉NYLDWYLQKSGQSPQLLIYLX₂₀SN |
| | | RASGVPDRFSGSGSGTDFTLKISRVEAEDVGFYYCMQX₂₁LX₂₂X₂₃PYTFGQGTKLEIK |
| Reference | NO: 24 | DIVMTQSPLSLPVTPGEPASISCRSSQSLLYSIGYNYLDWYLQKSGQSPQLLIYLGSNRASGV |
| | | PDRFSGSGSGTDFTLKISRVEAEDVGFYYCMQALQTPYTFGQGTKLEIK |
| AB-1 | NO: 25 | DIVMTQSPLSLPVTPGEPASISCRSSQSLDYSYGYNYLDWYLQKSGQSPQLLIYLASNRASGV |
| | | PDRFSGSGSGTDFTLKISRVEAEDVGFYYCMQSLSTPYTFGQGTKLEIK |
| AB-2 | NO: 26 | DIVMTQSPLSLPVTPGEPASISCRSSQSLDYSSGYNYLDWYLQKSGQSPQLLIYLASNRASGV |
| | | PDRFSGSGSGTDFTLKISRVEAEDVGFYYCMQSLQTPYTFGQGTKLEIK |
| AB-3 | NO: 27 | DIVMTQSPLSLPVTPGEPASISCRSSQSLLYSTGYNYLDWYLQKSGQSPQLLIYLGSNRASGV |
| | | PDRFSGSGSGTDFTLKISRVEAEDVGFYYCMQSLSTPYTFGQGTKLEIK |
| AB-4 | NO: 28 | DIVMTQSPLSLPVTPGEPASISCRSSQSLLYSTGYNYLDWYLQKSGQSPQLLIYLASNRASGV |
| | | PDRFSGSGSGTDFTLKISRVEAEDVGFYYCMQALSTPYTFGQGTKLEIK |
| AB-5 | NO: 29 | DIVMTQSPLSLPVTPGEPASISCRSSQSLDYSIGYNYLDWYLQKSGQSPQLLIYLASNRASGV |
| | | PDRFSGSGSGTDFTLKISRVEAEDVGFYYCMQALQTPYTFGQGTKLEIK |
| AB-6 | NO: 30 | DIVMTQSPLSLPVTPGEPASISCRSSQSLLYSIGYNYLDWYLQKSGQSPQLLIYLASNRASGV |
| | | PDRFSGSGSGTDFTLKISRVEAEDVGFYYCMQSLQTPYTFGQGTKLEIK |
| AB-7 | NO: 31 | DIVMTQSPLSLPVTPGEPASISCRSSQSLLYSEGYNYLDWYLQKSGQSPQLLIYLASNRASGV |
| | | PDRFSGSGSGTDFTLKISRVEAEDVGFYYCMQTLKTPYTFGQGTKLEIK |
| AB-8 | NO: 32 | DIVMTQSPLSLPVTPGEPASISCRSSQSLLYSEGYNYLDWYLQKSGQSPQLLIYLASNRASGV |
| | | PDRFSGSGSGTDFTLKISRVEAEDVGFYYCMQSLTTPYTFGQGTKLEIK |
| AB-9 | NO: 33 | DIVMTQSPLSLPVTPGEPASISCRSSQSLLYSLGYNYLDWYLQKSGQSPQLLIYLASNRASGV |
| | | PDRFSGSGSGTDFTLKISRVEAEDVGFYYCMQSLQTPYTFGQGTKLEIK |
| AB-10 | NO: 34 | DIVMTQSPLSLPVTPGEPASISCRSSQSLLYSVGKNYLDWYLQKSGQSPQLLIYLASNRASGV |
| | | PDRFSGSGSGTDFTLKISRVEAEDVGFYYCMQDLFHPYTFGQGTKLEIK |
| AB-11 | NO: 35 | DIVMTQSPLSLPVTPGEPASISCRSSQSLDYSYGYNYLDWYLQKSGQSPQLLIYLASNRASGV |
| | | PDRFSGSGSGTDFTLKISRVEAEDVGFYYCMQSLHTPYTFGQGTKLEIK |
| AB-12 | NO: 36 | DIVMTQSPLSLPVTPGEPASISCRSSQSLLYSQGYNYLDWYLQKSGQSPQLLIYLASNRASGV |
| | | PDRFSGSGSGTDFTLKISRVEAEDVGFYYCMQSLFTPYTFGQGTKLEIK |
| AB-13 | NO: 37 | DIVMTQSPLSLPVTPGEPASISCRSSQSLLYSHGYNYLDWYLQKSGQSPQLLIYLASNRASGV |
| | | PDRFSGSGSGTDFTLKISRVEAEDVGFYYCMQSLYTPYTFGQGTKLEIK |
| AB-14 | NO: 38 | DIVMTQSPLSLPVTPGEPASISCRSSQSLLYSHGYNYLDWYLQKSGQSPQLLIYLASNRASGV |
| | | PDRFSGSGSGTDFTLKISRVEAEDVGFYYCMQSLKTPYTFGQGTKLEIK |
| AB-15 | NO: 39 | DIVMTQSPLSLPVTPGEPASISCRSSQSLLYSRGYNYLDWYLQKSGQSPQLLIYLASNRASGV |
| | | PDRFSGSGSGTDFTLKISRVEAEDVGFYYCMQSLYTPYTFGQGTKLEIK |
| AB-16 | NO: 40 | DIVMTQSPLSLPVTPGEPASISCRSSQSLLYSIGYNYLDWYLQKSGQSPQLLIYLASNRASGV |
| | | PDRFSGSGSGTDFTLKISRVEAEDVGFYYCMQALQTPYTFGQGTKLEIK |
| AB-17 | NO: 41 | DIVMTQSPLSLPVTPGEPASISCRSSQSLLYSSGYNYLDWYLQKSGQSPQLLIYLASNRASGV |
| | | PDRFSGSGSGTDFTLKISRVEAEDVGFYYCMQSLQTPYTFGQGTKLEIK |

TABLE 3
CDR Amino Acid Sequences

| | | |
|---|---|---|
| Name | SEQ ID | Amino Acid Sequence |

HCDR1

| | | |
|---|---|---|
| Consensus | NO: 42 | GFX₁FX₂DYA |
| Reference, | NO: 43 | GFTFRDYA |
| AB-2, 3, 6-14 | | |
| AB-1, 4, 5 | NO: 44 | GFTFSDYA |
| AB-15 | NO: 45 | GFNFKDYA |

HCDR2

| | | |
|---|---|---|
| Consensus | NO: 46 | ISGSGX₃X₄X₅ |
| Reference, | NO: 47 | ISGSGGNT |
| AB-1, 3, 4 | | |
| AB-2, 5, 6, | NO: 48 | ISGSGSNT |
| 8, 9, 13, 15 | | |
| AB-7 | NO: 49 | ISGSGSTT |
| AB-10 | NO: 50 | ISGSGDKT |
| AB-11 | NO: 51 | ISGSGTNT |
| AB-12 | NO: 52 | ISGSGSNK |
| AB-14 | NO: 53 | ISGSGSNI |

HCDR3

| | | |
|---|---|---|
| Consensus | NO: 54 | AX₆DX₇X₈X₉X₁₀X₁₁X₁₂X₁₃ |
| | | PX₁₄YX₁₅GLD |
| Reference | NO: 55 | AKDRLSITIRPRYYGLD |
| AB-1 | NO: 56 | AKDALTIAIRPYYYGLD |
| AB-2 | NO: 57 | ARDRLTAAVRPYYYGLD |
| AB-3 | NO: 58 | AKDRLSKTIRPYYYGLD |
| AB-4 | NO: 59 | ARDRLTITIRPRYYGLD |
| AB-5 | NO: 60 | AKDRLTIAIRPYYYGLD |
| AB-6 | NO: 61 | AKDRLTTKVRPEYYGLD |
| AB-7 | NO: 62 | AKDRLTASVRPIYVGLD |
| AB-8 | NO: 63 | AKDRLTASVRPNYYGLD |
| AB-9 | NO: 64 | AKDRLTTAVRPSYYGLD |
| AB-10 | NO: 65 | ARDRLSSSARPVYVGLD |
| AB-11 | NO: 66 | AKDRLSVSTRPKYYGLD |
| AB-12 | NO: 67 | AKDRLTSRVRPYYYGLD |
| AB-13 | NO: 68 | AKDRWTSSVRPVYRGLD |
| AB-14 | NO: 69 | AKDRYTKSVRPVYRGLD |
| AB-15 | NO: 70 | AKDRWTSSVKPRYRGLD |

LCDR1

| | | |
|---|---|---|
| Consensus | NO: 71 | QSLX₁₇YSX₁₈GX₁₉NY |
| Reference, | NO: 72 | QSLLYSIGYNY |
| AB-6 | | |
| AB-1, 11 | NO: 73 | QSLDYSYGYNY |
| AB-2 | NO: 74 | QSLDYSSGYNY |
| AB-3, 4 | NO: 75 | QSLLYSTGYNY |
| AB-5 | NO: 76 | QSLDYSIGYNY |
| AB-7, 8 | NO: 77 | QSLLYSEGYNY |
| AB-9 | NO: 78 | QSLLYSLGYNY |
| AB-10 | NO: 79 | QSLLYSVGKNY |
| AB-12 | NO: 80 | QSLLYSQGYNY |
| AB-13, 14 | NO: 81 | QSLLYSHGYNY |
| AB-15 | NO: 82 | QSLLYSRGYNY |

LCDR2

| | | |
|---|---|---|
| Consensus | NO: 83 | LX₂₀S |
| Reference, | NO: 84 | LGS |
| AB-3 | | |
| AB-1-2, 4-15 | NO: 85 | LAS |

LCDR3

| | | |
|---|---|---|
| Consensus | NO: 86 | MQX₂₁LX₂₂X₂₃PY |
| Reference, | NO: 87 | MQALQTPY |
| AB-5 | | |
| AB-1, 3 | NO: 88 | MQSLSTPY |
| AB-2, 6, 9 | NO: 89 | MQSLQTPY |
| AB-4 | NO: 90 | MQALSTPY |
| AB-7 | NO: 91 | MQTLKTPY |
| AB-8 | NO: 92 | MQSLTTPY |
| AB-10 | NO: 93 | MQDLFHPY |
| AB-11 | NO: 94 | MQSLHTPY |
| AB-12 | NO: 95 | MQSLFTPY |
| AB-13, 15 | NO: 96 | MQSLYTPY |
| AB-14 | NO: 97 | MQSLKTPY |

TABLE 4
Constant Domain Amino Acid Sequences

| Name | SEQ ID | Heavy Chain Constant Domain Amino Acid Sequence |
|---|---|---|
| Fc_a | NO: 98 | ASTKGPSVFPLAPCSRSTSESTAALGCLVKDYFPEPVTVSWNSGALTSGVHTFPAVLQSS |
| (hIgG4 | | GLYSLSSVVTVPSSSLGTKTYTCNVDHKPSNTKVDKRVESKYGPPCPPCPAPEFLGGPSV |
| YTE) | | FLFPPKPKDTLYITREPEVTCVVVDVSQEDPEVQFNWYVDGVEVHNAKTKPREEQFNSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| Fc_b | NO: 99 | ASTKGPSVFPLAPCSRSTSESTAALGCLVKDYFPEPVTVSWNSGALTSGVHTFPAVLQSS |
| (hIgG4 | | GLYSLSSVVTVPSSSLGTKTYTCNVDHKPSNTKVDKRVESKYGPPCPPCPAPEFLGGPSV |
| LS) | | FLFPPKPKDTLMISRTPEVTCVVVDVSQEDPEVQFNWYVDGVEVHNAKTKPREEQENSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVLHEALHSHYTQKSLSLSLGK |
| Fc_c | NO: 100 | ASTKGPSVFPLAPCSRSTSESTAALGCLVKDYFPEPVTVSWNSGALTSGVHTFPAVLQSS |
| (hIgG4) | | GLYSLSSVVTVPSSSLGTKTYTCNVDHKPSNTKVDKRVESKYGPPCPPCPAPEFLGGPSV |
| | | FLFPPKPKDTLMISRTPEVTCVVVDVSQEDPEVQFNWYVDGVEVHNAKTKPREEQENSTY |
| | | RVVSVLTVLHQDWLNGKEYKCKVSNKGLPSSIEKTISKAKGQPREPQVYTLPPSQEEMTK |
| | | NQVSLTCLVKGFYPSDIAVEWESNGQPENNYKTTPPVLDSDGSFFLYSRLTVDKSRWQEG |
| | | NVFSCSVMHEALHNHYTQKSLSLSLGK |
| Fc_d | NO: 101 | ASTKGPSVFPLAPSSKSTSGGTAALGCLVKDYFPEPVTVSWNSGALTSGVHTFPAVLQSS |
| (hIgG1) | | GLYSLSSVVTVPSSSLGTQTYICNVNHKPSNTKVDKRVEPKSCDKTHTCPPCPAPELLGG |
| | | PSVFLFPPKPKDTLMISRTPEVTCVVVDVSHEDPEVKENWYVDGVEVHNAKTKPREEQYN |
| | | STYRVVSVLTVLHQDWLNGKEYKCKVSNKALPAPIEKTISKAKGQPREPQVYTLPPSREE |
| | | MTKNQVSLTCLVKGFYPSDIAVEWESNGQPENNYKTTPPVLDSDGSFFLYSKLTVDKSRW |
| | | QQGNVFSCSVMHEALHNHYTQKSLSLSPGK |
| Name | SEQ ID | Light Chain Constant Domain Amino Acid Sequence |
| K | NO: 102 | RTVAAPSVFIFPPSDEQLKSGTASVVCLLNNFYPREAKVQWKVDNALQSGNSQESVTEQD |
| | | SKDSTYSLSSTLTLSKADYEKHKVYACEVTHQGLSSPVTKSENRGEC |
| λ | NO: 103 | GQPKANPTVTLFPPSSEELQANKATLVCLISDFYPGAVTVAWKADGSPVKAGVETTKPSK |
| | | QSNNKYAASSYLSLTPEQWKSHRSYSCQVTHEGSTVEKTVAPTECS |

TABLE 5
Heavy Chain Amino Acid Sequences

| Name | SEQ ID | Amino Acid Sequence |
|---|---|---|
| Reference | NO: 104 | EVQLVESGGGLEQPGGSLRLSCAGSGFTFRDYAMTWVRQAPGKGLEWVSSISGSGGN |
| | | TYYADSVKGRFTISRDNSKNTLYLQMNSLRAEDTAVYYCAKDRLSITIRPRYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |

Human IgG Type hIgG4 YTE

| | | |
|---|---|---|
| AB-1a | NO: 105 | EVQLVESGGGLEQPGGSLRLSCAGSGFTFSDYAMTWVRQAPGKGLEWVSSISGSGGN |
| | | TYYADSVKGRFTISRDNSKNTLYLQMNSLRAEDTAVYYCAKDALTIAIRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-2a | NO: 106 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCARDRLTAAVRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-3a | NO: 107 | EVQLVESGGGLEQPGGSLRLSCAGSGFTFRDYAMTWVRQAPGKGLEWVSSISGSGGN |
| | | TYYADSVKGRFTISRDNSKNTLYLQMNSLRAEDTAVYYCAKDRLSKTIRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-4a | NO: 108 | EVQLVESGGGLEQPGGSLRLSCAGSGFTFSDYAMTWVRQAPGKGLEWVSSISGSGGN |
| | | TYYADSVKGRFTISRDNSKNTLYLQMNSLRAEDTAVYYCARDRLTITIRPRYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-4A | NO: 109 | EVQLVESGGGLEQPGGSLRLSCAGSGFTFSDYAMTWVRQAPGKGLEWVSSISGSGSN |
| | | TYYADSVKGRFTISRDNSKNTLYLQMNSLRAEDTAVYYCAKDRLTIAIRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-6a | NO: 110 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTTKVRPEYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-7a | NO: 111 | EVQLVESGGGLEQPGGSLRLSCAGSGFTFRDYAMTWVRQAPGKGLEWVSSISGSGST |
| | | TYYADSVKGRFTISRDNSKNTLYLQMNSLRAEDTAVYYCAKDRLTASVRPIYVGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-8a | NO: 112 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTASVRPNYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-9a | NO: 113 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTTAVRPSYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-10a | NO: 114 | EVQLVESGGGLEQPGGSLRLSCAGSGFTFRDYAMTWVRQAPGKGLEWVSSISGSGDK |
| | | TYYADSVKGRFTISRDNSKNTLYLQMNSLRAEDTAVYYCARDRLSSSARPVYVGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-11a | NO: 115 | EVQLVESGGGLEQPGGSLRLSCAGSGFTFRDYAMTWVRQAPGKGLEWVSSISGSGTN |
| | | TYYADSVKGRFTISRDNSKNTLYLQMNSLRAEDTAVYYCAKDRLSVSTRPKYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-12a | NO: 116 | EVQLVESGGGLEQPGGSLRLSCAGSGFTFRDYAMTWVRQAPGKGLEWVSSISGSGSN |
| | | KYYADSVKGRFTISRDNSKNTLYLQMNSLRAEDTAVYYCAKDRLTSRVRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-13a | NO: 117 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRWTSSVRPVYRGLDI |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLYITREPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |

Human IgG Type hIgG4 LS

| | | |
|---|---|---|
| AB-1b | NO: 118 | EVQLVESGGGLEQPGGSLRLSCAGSGFTFSDYAMTWVRQAPGKGLEWVSSISGSGGN |
| | | TYYADSVKGRFTISRDNSKNTLYLQMNSLRAEDTAVYYCAKDALTIAIRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-2b | NO: 119 | EVQLVESGGGLEQPGGSLRLSCAGSGFTERDYAMTWVRQAPGKGLEWVSSISGSGSN |
| | | TYYADSVKGRFTISRDNSKNTLYLQMNSLRAEDTAVYYCARDRLTAAVRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-3b | NO: 120 | EVQLVESGGGLEQPGGSLRLSCAGSGFTFRDYAMTWVRQAPGKGLEWVSSISGSGGN |
| | | TYYADSVKGRFTISRDNSKNTLYLQMNSLRAEDTAVYYCAKDRLSKTIRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-4b | NO: 121 | EVQLVESGGGLEQPGGSLRLSCAGSGFTFSDYAMTWVRQAPGKGLEWVSSISGSGGN |
| | | TYYADSVKGRFTISRDNSKNTLYLQMNSLRAEDTAVYYCARDRLTITIRPRYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-4B | NO: 122 | EVQLVESGGGLEQPGGSLRLSCAGSGFTFSDYAMTWVRQAPGKGLEWVSSISGSGSN |
| | | TYYADSVKGRFTISRDNSKNTLYLQMNSLRAEDTAVYYCAKDRLTIAIRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-6b | NO: 123 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTTKVRPEYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-7b | NO: 124 | EVQLVESGGGLEQPGGSLRLSCAGSGFTFRDYAMTWVRQAPGKGLEWVSSISGSGST |
| | | TYYADSVKGRFTISRDNSKNTLYLQMNSLRAEDTAVYYCAKDRLTASVRPIYVGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-8b | NO: 125 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTASVRPNYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-9b | NO: 126 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTTAVRPSYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQENWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-10b | NO: 127 | EVQLVESGGGLEQPGGSLRLSCAGSGFTFRDYAMTWVRQAPGKGLEWVSSISGSGDK |
| | | TYYADSVKGRFTISRDNSKNTLYLQMNSLRAEDTAVYYCARDRLSSSARPVYVGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-11b | NO: 128 | EVQLVESGGGLEQPGGSLRLSCAGSGFTFRDYAMTWVRQAPGKGLEWVSSISGSGTN |
| | | TYYADSVKGRFTISRDNSKNTLYLQMNSLRAEDTAVYYCAKDRLSVSTRPKYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-12b | NO: 129 | EVQLVESGGGLEQPGGSLRLSCAGSGFTFRDYAMTWVRQAPGKGLEWVSSISGSGSN |
| | | KYYADSVKGRFTISRDNSKNTLYLQMNSLRAEDTAVYYCAKDRLTSRVRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |
| AB-13b | NO: 130 | EVQLVESGGGLEQPGGSLRLSCAGSGFTERDYAMTWVRQAPGKGLEWVSSISGSGSN |
| | | TYYADSVKGRFTISRDNSKNTLYLQMNSLRAEDTAVYYCAKDRWTSSVRPVYRGLDI |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVLHEALHSHYTQKSLSLSLGK |

Human IgG Type hIgG4

| | | |
|---|---|---|
| AB-1c | NO: 131 | EVQLVESGGGLEQPGGSLRLSCAGSGFTFSDYAMTWVRQAPGKGLEWVSSISGSGGN |
| | | TYYADSVKGRFTISRDNSKNTLYLQMNSLRAEDTAVYYCAKDALTIAIRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-2c | NO: 132 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCARDRLTAAVRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-3c | NO: 133 | EVQLVESGGGLEQPGGSLRLSCAGSGFTFRDYAMTWVRQAPGKGLEWVSSISGSGGN |
| | | TYYADSVKGRFTISRDNSKNTLYLQMNSLRAEDTAVYYCAKDRLSKTIRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-4c | NO: 134 | EVQLVESGGGLEQPGGSLRLSCAGSGFTFSDYAMTWVRQAPGKGLEWVSSISGSGGN |
| | | TYYADSVKGRFTISRDNSKNTLYLQMNSLRAEDTAVYYCARDRLTITIRPRYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-5c | NO: 135 | EVQLVESGGGLEQPGGSLRLSCAGSGFTFSDYAMTWVRQAPGKGLEWVSSISGSGSN |
| | | TYYADSVKGRFTISRDNSKNTLYLQMNSLRAEDTAVYYCAKDRLTIAIRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-6c | NO: 136 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTTKVRPEYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-7c | NO: 137 | EVQLVESGGGLEQPGGSLRLSCAGSGFTFRDYAMTWVRQAPGKGLEWVSSISGSGST |
| | | TYYADSVKGRFTISRDNSKNTLYLQMNSLRAEDTAVYYCAKDRLTASVRPIYVGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-8c | NO: 138 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTASVRPNYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-9c | NO: 139 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTTAVRPSYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-10c | NO: 140 | EVQLVESGGGLEQPGGSLRLSCAGSGFTFRDYAMTWVRQAPGKGLEWVSSISGSGDK |
| | | TYYADSVKGRFTISRDNSKNTLYLQMNSLRAEDTAVYYCARDRLSSSARPVYVGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-11c | NO: 141 | EVQLVESGGGLEQPGGSLRLSCAGSGFTFRDYAMTWVRQAPGKGLEWVSSISGSGTN |
| | | TYYADSVKGRFTISRDNSKNTLYLQMNSLRAEDTAVYYCAKDRLSVSTRPKYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-12c | NO: 142 | EVQLVESGGGLEQPGGSLRLSCAGSGFTFRDYAMTWVRQAPGKGLEWVSSISGSGSN |
| | | KYYADSVKGRFTISRDNSKNTLYLQMNSLRAEDTAVYYCAKDRLTSRVRPYYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-13c | NO: 143 | EVQLVESGGGLEQPGGSLRLSCAGSGFTERDYAMTWVRQAPGKGLEWVSSISGSGSN |
| | | TYYADSVKGRFTISRDNSKNTLYLQMNSLRAEDTAVYYCAKDRWTSSVRPVYRGLDI |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-14c | NO: 144 | EVQLVESGGGLEQPGGSLRLSCAGSGFTFRDYAMTWVRQAPGKGLEWVSSISGSGSN |
| | | IYYADSVKGRFTISRDNSKNTLYLQMNSLRAEDTAVYYCAKDRYTKSVRPVYRGLDI |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-15c | NO: 145 | EVQLVESGGGLEQPGGSLRLSCAGSGFNFKDYAMTWVRQAPGKGLEWVSSISGSGSN |
| | | TYYADSVKGRFTISRDNSKNTLYLQMNSLRAEDTAVYYCAKDRWTSSVKPRYRGLDI |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-16c | NO: 146 | EVQLVESGGGLEQPGGSLRLSCAGSGFTFSDYAMTWVRQAPGKGLEWVSSISGSGGN |
| | | TYYADSVKGRFTISRDNSKNTLYLQMNSLRAEDTAVYYCAKDALSITIRPRYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |
| AB-17c | NO: 147 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTAAVRPRYYGLDV |
| | | WGQGTTVTVSSASTKGPSVFPLAPCSRSTSESTAALGCLVKDYFPEPVTVSWNSGAL |
| | | TSGVHTFPAVLQSSGLYSLSSVVTVPSSSLGTKTYTCNVDHKPSNTKVDKRVESKYG |
| | | PPCPPCPAPEFLGGPSVFLFPPKPKDTLMISRTPEVTCVVVDVSQEDPEVQFNWYVD |
| | | GVEVHNAKTKPREEQFNSTYRVVSVLTVLHQDWLNGKEYKCKVSNKGLPSSIEKTIS |
| | | KAKGQPREPQVYTLPPSQEEMTKNQVSLTCLVKGFYPSDIAVEWESNGQPENNYKTT |
| | | PPVLDSDGSFFLYSRLTVDKSRWQEGNVFSCSVMHEALHNHYTQKSLSLSLGK |

Human IgG Type hIgG1

| | | |
|---|---|---|
| AB-1d | NO: 148 | EVQLVESGGGLEQPGGSLRLSCAGSGFTFSDYAMTWVRQAPGKGLEWVSSISGSGGN |
| | | TYYADSVKGRFTISRDNSKNTLYLQMNSLRAEDTAVYYCAKDALTIAIRPYYYGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-2d | NO: 149 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCARDRLTAAVRPYYYGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-3d | NO: 150 | EVQLVESGGGLEQPGGSLRLSCAGSGFTFRDYAMTWVRQAPGKGLEWVSSISGSGGN |
| | | TYYADSVKGRFTISRDNSKNTLYLQMNSLRAEDTAVYYCAKDRLSKTIRPYYYGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-4d | NO: 151 | EVQLVESGGGLEQPGGSLRLSCAGSGFTFSDYAMTWVRQAPGKGLEWVSSISGSGGN |
| | | TYYADSVKGRFTISRDNSKNTLYLQMNSLRAEDTAVYYCARDRLTITIRPRYYGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-5d | NO: 152 | EVQLVESGGGLEQPGGSLRLSCAGSGFTFSDYAMTWVRQAPGKGLEWVSSISGSGSN |
| | | TYYADSVKGRFTISRDNSKNTLYLQMNSLRAEDTAVYYCAKDRLTIAIRPYYYGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-6d | NO: 153 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTTKVRPEYYGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-7d | NO: 154 | EVQLVESGGGLEQPGGSLRLSCAGSGFTFRDYAMTWVRQAPGKGLEWVSSISGSGST |
| | | TYYADSVKGRFTISRDNSKNTLYLQMNSLRAEDTAVYYCAKDRLTASVRPIYVGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-8d | NO: 155 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTASVRPNYYGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-9d | NO: 156 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRLTTAVRPSYYGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-10d | NO: 157 | EVQLVESGGGLEQPGGSLRLSCAGSGFTFRDYAMTWVRQAPGKGLEWVSSISGSGDK |
| | | TYYADSVKGRFTISRDNSKNTLYLQMNSLRAEDTAVYYCARDRLSSSARPVYVGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-11d | NO: 158 | EVQLVESGGGLEQPGGSLRLSCAGSGFTFRDYAMTWVRQAPGKGLEWVSSISGSGTN |
| | | TYYADSVKGRFTISRDNSKNTLYLQMNSLRAEDTAVYYCAKDRLSVSTRPKYYGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-12d | NO: 159 | EVQLVESGGGLEQPGGSLRLSCAGSGFTFRDYAMTWVRQAPGKGLEWVSSISGSGSN |
| | | KYYADSVKGRFTISRDNSKNTLYLQMNSLRAEDTAVYYCAKDRLTSRVRPYYYGLDV |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |
| AB-13d | NO: 160 | EVQLVESGGGLEQPGGSLRLSCAGSGFTFRDYAMTWVRQAPGKGLEWVSSISGSGSN |
| | | TYYADSVKGRFTISRDNSKNTLYLQMNSLRAEDTAVYYCAKDRWTSSVRPVYRGLDI |
| | | WGQGTTVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKRVEPKSC |
| | | DKTHTCPPCPAPELLGGPSVFLFPPKPKDTLMISRTPEVTCVVVDVSHEDPEVKENW |
| | | YVDGVEVHNAKTKPREEQYNSTYRVVSVLTVLHQDWLNGKEYKCKVSNKALPAPIEK |
| | | TISKAKGQPREPQVYTLPPSREEMTKNQVSLTCLVKGFYPSDIAVEWESNGQPENNY |
| | | KTTPPVLDSDGSFFLYSKLTVDKSRWQQGNVFSCSVMHEALHNHYTQKSLSLSPGK |

TABLE 6
Light Chain Amino Acid Sequences

| Name | SEQ ID | Amino Acid Sequence |
|---|---|---|
| Reference | NO: 161 | DIVMTQSPLSLPVTPGEPASISCRSSQSLLYSIGYNYLDWYLQKSGQSPQLLIYLGSNRAS |
| | | GVPDRFSGSGSGTDFTLKISRVEAEDVGFYYCMQALQTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-1 | NO: 162 | DIVMTQSPLSLPVTPGEPASISCRSSQSLDYSYGYNYLDWYLQKSGQSPQLLIYLASNRAS |
| | | GVPDRFSGSGSGTDFTLKISRVEAEDVGFYYCMQSLSTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-2 | NO: 163 | DIVMTQSPLSLPVTPGEPASISCRSSQSLDYSSGYNYLDWYLQKSGQSPQLLIYLASNRAS |
| | | GVPDRESGSGSGTDFTLKISRVEAEDVGFYYCMQSLQTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-3 | NO: 164 | DIVMTQSPLSLPVTPGEPASISCRSSQSLLYSTGYNYLDWYLQKSGQSPQLLIYLGSNRAS |
| | | GVPDRFSGSGSGTDFTLKISRVEAEDVGFYYCMQSLSTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-4 | NO: 165 | DIVMTQSPLSLPVTPGEPASISCRSSQSLLYSTGYNYLDWYLQKSGQSPQLLIYLASNRAS |
| | | GVPDRFSGSGSGTDFTLKISRVEAEDVGFYYCMQALSTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-5 | NO: 166 | DIVMTQSPLSLPVTPGEPASISCRSSQSLDYSIGYNYLDWYLQKSGQSPQLLIYLASNRAS |
| | | GVPDRFSGSGSGTDFTLKISRVEAEDVGFYYCMQALQTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-6 | NO: 167 | DIVMTQSPLSLPVTPGEPASISCRSSQSLLYSIGYNYLDWYLQKSGQSPQLLIYLASNRAS |
| | | GVPDRFSGSGSGTDFTLKISRVEAEDVGFYYCMQSLQTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-7 | NO: 168 | DIVMTQSPLSLPVTPGEPASISCRSSQSLLYSEGYNYLDWYLQKSGQSPQLLIYLASNRAS |
| | | GVPDRFSGSGSGTDFTLKISRVEAEDVGFYYCMQTLKTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-8 | NO: 169 | DIVMTQSPLSLPVTPGEPASISCRSSQSLLYSEGYNYLDWYLQKSGQSPQLLIYLASNRAS |
| | | GVPDRFSGSGSGTDFTLKISRVEAEDVGFYYCMQSLTTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-9 | NO: 170 | DIVMTQSPLSLPVTPGEPASISCRSSQSLLYSLGYNYLDWYLQKSGQSPQLLIYLASNRAS |
| | | GVPDRFSGSGSGTDFTLKISRVEAEDVGFYYCMQSLQTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-10 | NO: 171 | DIVMTQSPLSLPVTPGEPASISCRSSQSLLYSVGKNYLDWYLQKSGQSPQLLIYLASNRAS |
| | | GVPDRFSGSGSGTDFTLKISRVEAEDVGFYYCMQDLFHPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-11 | NO: 172 | DIVMTQSPLSLPVTPGEPASISCRSSQSLDYSYGYNYLDWYLQKSGQSPQLLIYLASNRAS |
| | | GVPDRFSGSGSGTDFTLKISRVEAEDVGFYYCMQSLHTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-12 | NO: 173 | DIVMTQSPLSLPVTPGEPASISCRSSQSLLYSQGYNYLDWYLQKSGQSPQLLIYLASNRAS |
| | | GVPDRFSGSGSGTDFTLKISRVEAEDVGFYYCMQSLFTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-13 | NO: 174 | DIVMTQSPLSLPVTPGEPASISCRSSQSLLYSHGYNYLDWYLQKSGQSPQLLIYLASNRAS |
| | | GVPDRFSGSGSGTDFTLKISRVEAEDVGFYYCMQSLYTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-14 | NO: 175 | DIVMTQSPLSLPVTPGEPASISCRSSQSLLYSHGYNYLDWYLQKSGQSPQLLIYLASNRAS |
| | | GVPDRFSGSGSGTDFTLKISRVEAEDVGFYYCMQSLKTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-15 | NO: 176 | DIVMTQSPLSLPVTPGEPASISCRSSQSLLYSRGYNYLDWYLQKSGQSPQLLIYLASNRAS |
| | | GVPDRFSGSGSGTDFTLKISRVEAEDVGFYYCMQSLYTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-16 | NO: 177 | DIVMTQSPLSLPVTPGEPASISCRSSQSLLYSIGYNYLDWYLQKSGQSPQLLIYLASNRAS |
| | | GVPDRFSGSGSGTDFTLKISRVEAEDVGFYYCMQALQTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |
| AB-17 | NO: 178 | DIVMTQSPLSLPVTPGEPASISCRSSQSLLYSSGYNYLDWYLQKSGQSPQLLIYLASNRAS |
| | | GVPDRFSGSGSGTDFTLKISRVEAEDVGFYYCMQSLQTPYTFGQGTKLEIKRTVAAPSVFI |
| | | FPPSDEQLKSGTASVVCLLNNFYPREAKVQWKVDNALQSGNSQESVTEQDSKDSTYSLSST |
| | | LTLSKADYEKHKVYACEVTHQGLSSPVTKSENRGEC |

### Computational Definition and Verification of Sequences

Applicant, through computational design and experimental validation, has facilitated an understanding of structure and function interrelation of anti-IL-4Rα antibodies and herein provides the skilled artisan the means to use such understanding. For example, the Application provides Computer Program Listing Appendices (also referred to herein as Appendices A, B, and C, respectively) that can be used to both evaluate (score) a given sequence or generate a sequence having a score that, when evaluated, is above a given threshold. These Appendices include a Potts model of polypeptides provided by embodiments.

A statistical machine learning model, according to some embodiments, was developed to predict functional properties of polypeptides, e.g., antibody binding to an interleukin-4 receptor alpha (IL-4Rα) target. An example model was trained on experimental binding data and uses a Potts statistical model to make predictions about, e.g., whether novel antibody sequences will bind to IL-4Rα.

For model selection, multiple example candidate model architectures were evaluated as described below, and a Potts model was selected based on optimal performance across multiple metrics:
a) Linear model (224 parameters)
b) Uniform model (224 parameters)
c) Positive Potts model (169,408 parameters)
d) Mixture model (231,492 parameters)
e) Joint model (246,717 parameters)
f) Ratio model (383,128 parameters)

The selected Potts model, according to some embodiments, demonstrates strong predictive performance according to the following example model performance metrics:
a) Area Under Curve (probability of distinguishing one randomly selected positive instance from one randomly selected negative instance): 0.831
b) Accuracy: 76.2%
c) Precision (true positives/(true positives+false positives)): 60.5%
d) Recall (true positives/(true positives+false negatives)): 84.3%
e) F1 Score (harmonic mean of precision and recall): 0.704 [2*((0.605*0.843)/(0.605+0.843))=˜0.704]
f) Binding Threshold: −27.87 (model score)

In some embodiments, the model may utilize an example dataset that is sourced from experimental binding affinity data from antibody variants for a target of interleukin-4 receptor alpha (IL-4Rα). The dataset may include 378 example antibody sequences with binding affinities measured to determine binding dissociation constants (K_{D} values). An example binding threshold of log (K_{D})=0.375 may be established, i.e., sequences with log (K_{D})<0.375 may be classified as “binding.” Other binding threshold values are also suitable.

The model architecture may be a positive Potts model that employes a statistical physics-based approach for sequence modeling. 169,408 example model parameters may be used. The model may take as input variable regions from heavy chain (V_{H}) and light chain (V_{L}) antibody sequences. An example V_{H} region input may include 25 amino acid positions, while an example V_{L} region input may include 17 amino acid positions, resulting in a total of 42 example amino acid positions for analysis.

In some embodiments, for a statistical approach, the model may leverage a Potts statistical framework that analyzes single amino acid site effects. For example, the framework may capture a preference for specific amino acids at each amino acid position in a given sequence of a polypeptide. The framework may further analyze pairwise interactions between amino acids at different positions. For example, the framework may model epistatic effects between amino acid positions. The framework may further perform cross-entropy scoring. For example, the framework may use negative log-pseudolikelihood for sequence evaluation.

In certain embodiments, the model may utilize a scoring mechanism where higher scores indicate, e.g., a greater likelihood of binding. According to an embodiment, a threshold value of approximately-27.87 for negative log-pseudolikelihood may separate, e.g., binding from non-binding sequences. Other threshold values may also be used. As described herein, a scoring mechanism according to some embodiments may incorporate both individual amino acid preferences and inter-position correlations.

It should be noted that in certain embodiments, a Potts model may not directly predict K_{D} or log (K_{D}) values, but rather may generate a score (e.g., a negative log-pseudolikelihood) that correlates with K_{D} or log (K_{D}) values. According to some embodiments, a threshold for a Potts model score may be specified so as to maximally correctly classify a set of binders (e.g., log (K_{D})<0.375) from nonbinders (e.g., log (K_{D})>=0.375) using training data. In certain embodiments, a threshold for a Potts model score may correspond either exactly or approximately to a log (K_{D}) value of 0.375.

The Computer Program Listing Appendices are referred to as Appendix A (score_concise.txt), Appendix B (score.txt), and Appendix C (fit_model.txt), which are herein incorporated by reference in their entireties. A person having ordinary skill in the art can recognize that Appendix A or Appendix B can respectively be renamed “score_concise.py” or “score.py” to be executed using standard libraries (e.g., math, sys, etc.) and without external dependencies in a Python environment (e.g., Python 3.x), compiler, or other equivalent environment that can run Python scripts, and that Appendix C can be renamed “fit_model.etab” and can be loaded by the script of Appendix A or Appendix B. A person having ordinary skill in the art can recognize that, in some embodiments, a Python environment may also run or compile the script of Appendix A or Appendix B without renaming. Other known programming languages and/or scripting environments are also suitable. When running the score_concise.py or score.py file, two sequences (or one combined sequence) can be inputted. In embodiments, the below description further describes the scripts of Appendices A and B and the model file of Appendix C.

The Potts model may be represented by a table of trained model parameters. The table may include single amino acid site parameters and pairwise amino acid parameters. For the single site parameters, the table may include a position of a residue (e.g., amino acid), the residue, and a value (e.g., a floating-point value) associated with the residue at that position. For the pairwise parameters, the table may include two positions, two residues, and a value (e.g., a floating-point value) associated with the two residues at the respective positions. The model parameters may be stored in the plaintext model file fit_model.etab.

In some embodiments, a script (e.g., Appendix A or Appendix B) may be employed to determine whether a sequence pair (e.g., V_{H} and V_{L}—although, for clarity, the sequence pair, in different embodiments, can be separate polypeptide chains or, in some embodiments, a single polypeptide chain, e.g., an scFV) is claimed under the model—i.e., whether the sequence pair, when scored by the model, is above a threshold. The script may be referred to as a computationally binding optimized (CBO) script. The script may take full-length sequences as inputs, e.g., V_{H} and V_{L} sequences provided as complete antibody sequences. In an embodiment, the sequences may be in standard single-letter amino acid code format; other known formats are also suitable. To continue, the script may then confirm that the input sequences match specific constant region templates. Shown below are example sequence templates according to an embodiment:

Heavy Chain Template
EVQLVESGGGLEQPGGSLRLSCAGSGXXXXXXAMTWVRQ
APGKGLEWVSSIXGSGXXXYYADSVKGRFTISRDNSKNT
LYLQMNSLRAEDTAVYYCXXXXXXXXXXPXYXGXXXWGQ
GTTVTVSS
Light Chain Template
DIVMTQSPLSLPVTPGEPASISCRSSXSXXYXXGXXYLD
WYLQKSGQSPQLLIYXXXNRASGVPDRFSGSGSGTDFTL
KISRVEAEDVGFYYCXXXXXXPXTFGQGTKLEIK

where ‘x’ denotes variable positions that are analyzed by the model.

In certain embodiments, a script, e.g., score_concise.py (Appendix A), may be used in either manner shown below:

### Command Line Usage

python score_concise.py “V_{H} SEQUENCE” “VL_SEQUENCE”

### Python Application Programming Interface (API) Usage

from score_concise import is_binding_antibody
# Returns True if predicted to bind, False otherwise
result=is_binding_antibody (vh_sequence, vl_sequence)

The script may construct two parameter hash tables for single site parameters and pairwise parameters, respectively, by loading an existing model file (e.g., Appendix C) and extracting single site amino acid positions/amino acid position pairs, amino acid identifiers, and corresponding values for each. For the single site parameters table, the script may extract one position, one residue identifier, and one value associated with the residue at the position. For the pairwise parameters table, the script may extract a first position, a second position, a first residue identifier, a second residue identifier, and a value associated with the first residue at the first position and the second residue at the second position.

The two sequences, the V_{H} sequence and V_{L} sequence, input to the script may then be aligned. The alignment may confirm that the V_{H} sequence and V_{L} sequence are the same length as template sequence(s) and that given sections of both input sequences are the same as the template sequence(s). Once confirmed, residues from both input sequences at specified positions may then be extracted to form a trimmed and concatenated sequence. In turn, a sequence score may be determined for the given concatenated sequence using the two parameter hash tables.

The following steps describe some embodiments of determining the sequence score. The is_binding_antibody( ) function of the script of Appendix A may call the calculate_binding_score( ) function to determine and return a score for the sequence. The return value of the calculate_binding_score( ) function may represent a sum of cross-entropy loss at each amino acid position in the concatenated sequence. If the sequence score is greater than the threshold, which in some embodiments is approximately-27.87, then the sequences meet the required parameters (e.g., are confirmed/verified by the Potts model) and the is_binding_antibody( ) function may return true. Otherwise, the function may return false and the sequence pair is not confirmed by the model. When the is_binding_antibody( ) function returns true, this may indicate that the sequence pair is suitable for the given purpose (e.g., a purpose related to binding to human IL-4Rα).

In some embodiments, the script of Appendix A may define a sequence scoring function (calculate_binding_score) that takes as inputs a sequence and a pair of parameter hash tables constructed from a model file (e.g., Appendix C). This function may determine a plurality of energy scores, including single amino acid energy scores and pairwise amino acid energy scores, for each amino acid position in the sequence. For a given amino acid position in the sequence, the calculate_binding_score function may use a Potts model to determine a single amino acid energy score and a sum of pairwise amino acid energy scores for each possible residue (e.g., amino acid) at the position. As described above, the function may check each possible residue at each position requested. A person of ordinary skill in the art can recognize that the script can replace residues at each given position (as in the script of Appendix A). In some embodiments, a similar script could add residues into the sequence. Continuing with the calculate_binding_score function, the single amino acid energy score and the sum of pairwise amino acid energy scores may be summed and added to an array of amino acid energies. A maximum energy value in the array of amino acid energies may then be determined and the array may be further used to generate a partition function. In some embodiments, a “softmax” function or “LogSumExp” (also called “RealSoftMax”) function may be applied to normalize the amino acid energies for numerical stability; other known normalization techniques are also suitable. A cross-entropy metric (e.g., cross-entropy loss) may be determined for the given amino acid position based on an amino acid at the amino acid position, the maximum energy value, and the partition function. Other known metrics are also suitable. In turn, the calculate_binding_score function may sum the cross-entropy metrics at each amino acid position and use the resulting sum to generate a sequence score, which score may be returned by the function.

In some embodiments, the script of Appendix A may define a load_model_parameters( ) function to load Potts model parameters from a model file (e.g., Appendix C). The file may be a fit_model.etab file in plaintext “.etab” format; other model file formats are also suitable, including text-based and binary formats, for non-limiting examples. To continue, each line in the file having three items may be unpacked by the load_model_parameters( ) function and converted into an entry in a first hash table for single amino acid site parameters. Each line in the file having five items may be unpacked and converted into an entry of a second hash table for pairwise amino acid parameters. The two hash tables may then be returned by the load_model_parameters( ) function.

The above steps and functions relate to examples of a “computationally binding optimized” (CBO) sequence, amino acid sequence, or polypeptide sequence. Such a sequence is a sequence that, when inputted into the above CBO script of Appendix A, returns a value of true. A person having ordinary skill in the art can recognize that the script can be implemented in other ways and/or with other series of steps, e.g., in the form of the script of Appendix B. A person having ordinary skill in the art can also recognize that other functionally equivalent tables can be used in place of the table of Appendix C. However, the provided Python scripts and table, when executed by a processor, are configured to output whether a given polypeptide sequence is a CBO sequence. Therefore, any sequence that, when analyzed by the scripts, results in a Boolean “true” output, is considered a CBO sequence. Any sequence that, when run by the scripts, results in a Boolean “false” output, is considered excluded from the group of the CBO sequences. Therefore, in some embodiments, the scripts of Appendix A and Appendix B themselves do not determine CBO sequences, but instead are tools to confirm whether a given sequence is a CBO sequence or not. When a CBO sequence is an amino acid sequence, the corresponding molecule having the amino acid sequence is referred to as a CBO polypeptide.

In certain embodiments, a similar script to Appendix A or Appendix B can return calculated scores of a given or multiple sequences in addition to or separate from a Boolean value representing the scores exceeding a threshold.

In some embodiments, a similar script to Appendix A or Appendix B can return one or more generated sequences from a Potts model. To generate sequences, Markov chain Monte Carlo (MCMC) sampling can be performed on the model. The resulting samples can then be reintegrated into a sequence template. In some embodiments, a brute force technique can be applied using the calculate_binding_score function of Appendix A.

Shown below are example predictions by the script of Appendix A (score_concise.py) according to an embodiment:

Binding Prediction (True)
python score concise.py
“EVQLVESGGGLEQPGGSLRLSCAGSGQLDSSYAMTWVRQAPGKGLEWVSSINGSGLASYYADS
VKGRFTISRDNSKNTLYLQMNSLRAEDTAVYYCMQSLQTYFTFPRYDGYSSWGQGTTVTVSS”
“DIVMTQSPLSLPVTPGEPASISCRSSNSTAYRDGRLYLDWYLQKSGQSPQLLIYTAANRASGV
PDRFSGSGSGTDFTLKISRVEAEDVGFYYCVRYYLDPVTFGQGTKLEIK”
Score = −19.85 (>threshold −27.87) → Predicted to bind
Non-binding Prediction (False)
python score concise.py
“EVQLVESGGGLEQPGGSLRLSCAGSGQLDSYYAMTWVRQAPGKGLEWVSSINGSGLASYYADS
VKGRFTISRDNSKNTLYLQMNSLRAEDTAVYYCMQSLSTYFTFPSYDGYSGWGQGTTVTVSS”
“DIVMTQSPLSLPVTPGEPASISCRSSNSTAYKDGALYLDWYLQKSGQSPQLLIYTIANRASGV
PDRFSGSGSGTDFTLKISRVEAEDVGFYYCIRYYLDPVTFGQGTKLEIK”
Score = −28.37 (<threshold −27.87) → Predicted not to bind

The Potts model, according to some embodiments, was validated using multiple example techniques as described below:
a) Threshold optimization: Youden's J statistic was used to find an optimal discrimination threshold.
b) Cross-validation: Model performance was assessed on held-out test sequences.
c) Template matching: Templates were utilized to ensure sequence compatibility before prediction.

Shown below are example validation results for the Potts model according to some embodiments:
a) True Positive Rate: 84.3% (correctly identifies binding antibodies)
b) False Positive Rate: 27.9% (incorrectly predicts binding for non-binding antibodies)
c) True Negative Rate: 72.1% (correctly identifies non-binding antibodies)
d) False Negative Rate: 15.7% (incorrectly predicts non-binding for binding antibodies)

In some embodiments, the scripts of Appendix A and Appendix B may depend on sequence templates by requiring input sequences to match specific constant region templates. The Potts model, according to some embodiments, may have a training data scope based on training the model on specific IL-4Rα binding data without validating generalization to other potential targets. Experimental validation may be performed on computational predictions generated by a model to confirm the predictions with experimental binding assays. The 27.9% false positive rate according to some embodiments discussed herein may indicate that approximately 1 in 4 predicted binders may not actually bind.

A Potts model according to an embodiment was applied to an example set of 17 test sequences, resulting in 12 of the 17 sequences (approx. 70.6%) being predicted as binding. Table 10 shows the 12 sequences predicted as binding and their corresponding log (K_{D}) scores.

TABLE 10
Example Variable Sequences and log(KD) Scores

| Name | variable_sequence | SEQ ID | log(K_{D}) |
|---|---|---|---|
| AB-2 | QLDSSYNLASMQSLQTYFTFRDYSS | NO: 179 | 0.8242813248742272 |
| | NTARDRLTAAVRYYLDV | | |
| AB-4 | QLLSTYNLASMQALSTYFTESDYSG | NO: 180 | −0.2485023666347192 |
| | NTARDRLTITIRRYLDV | | |
| AB-5 | QLDSIYNLASMQALQTYFTESDYSS | NO: 181 | 0.3745434711785539 |
| | NTAKDRLTIAIRYYLDV | | |
| AB-6 | QLLSIYNLASMQSLQTYFTFRDYSS | NO: 182 | −0.9677168756800749 |
| | NTAKDRLTTKVREYLDV | | |
| AB-7 | QLLSEYNLASMQTLKTYFTFRDYSS | NO: 183 | 0.06142641560348105 |
| | TTAKDRLTASVRIVLDV | | |
| AB-8 | QLLSEYNLASMQSLTTYFTERDYSS | NO: 184 | −0.16875134833306613 |
| | NTAKDRLTASVRNYLDV | | |
| AB-9 | QLLSLYNLASMQSLQTYFTFRDYSS | NO: 185 | −0.1982001389636858 |
| | NTAKDRLTTAVRSYLDV | | |
| AB-10 | QLLSVKNLASMQDLFHYFTFRDYSD | NO: 186 | 2.710819555334054 |
| | KTARDRLSSSARVVLDV | | |
| AB-11 | QLDSYYNLASMQSLHTYFTFRDYST | NO: 187 | −0.03944434823408831 |
| | NTAKDRLSVSTRKYLDV | | |
| AB-12 | QLLSQYNLASMQSLFTYFTERDYSS | NO: 188 | −1.0086993025983533 |
| | NKAKDRLTSRVRYYLDV | | |
| AB-13 | QLLSHYNLASMQSLYTYFTFRDYSS | NO: 189 | −0.9254219401526529 |
| | NTAKDRWTSSVRVRLDI | | |
| AB-17 | QLLSSYNLASMQSLQTYFTERDYSS | NO: 190 | 2.911454752932164 |
| | NTAKDRLTAAVRRYLDV | | |

### Example Method Embodiment

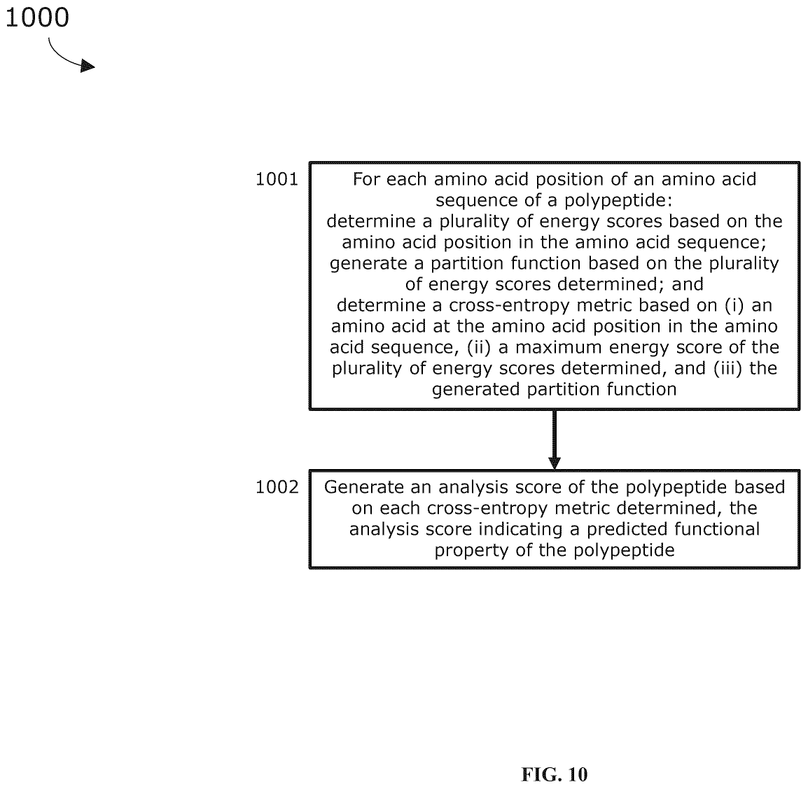

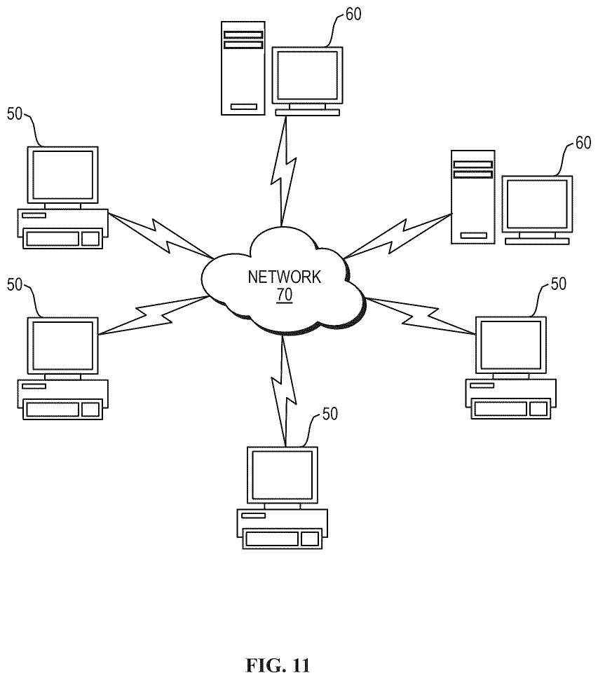

FIG. 10 is a flowchart of a method 1000 for predicting a functional property of a polypeptide according to an embodiment. The method 1000 is computer-implemented and may be implemented using any computing device, e.g., a processor, or combination of computing devices known to those of skill in the art.

The method 1000 begins at step 1001 by, via a computationally binding optimized (CBO) model, for each amino acid position of an amino acid sequence of the polypeptide: (1) determining a plurality of energy scores based on the amino acid position in the amino acid sequence, (2) generating a partition function based on the plurality of energy scores determined, and (3) determining a cross-entropy metric (e.g., cross-entropy loss) based on (i) an amino acid at the amino acid position in the amino acid sequence, (ii) a maximum energy score of the plurality of energy scores determined, and (iii) the generated partition function. At step 1002, an analysis score of the polypeptide is generated based on each cross-entropy metric determined. The analysis score indicates a predicted functional property of the polypeptide. The polypeptide comprises an immunoglobulin heavy chain variable domain (V_{H}) comprising an amino acid sequence having at least 90% sequence identity to SEQ ID NO:18 and an immunoglobulin light chain variable domain (V_{L}) comprising an amino acid sequence having at least 90% sequence identity to SEQ ID NO:37. The polypeptide does not comprise a V_{H} comprising an amino acid sequence having 100% sequence identity to SEQ ID NO:5 and a V_{L} comprising an amino acid sequence having 100% sequence identity to SEQ ID NO:24.

In some example embodiments, the polypeptide comprises an immunoglobulin heavy chain variable domain (V_{H}) comprising an amino acid sequence having at least 70% (e.g., at least 75%, at least 80%, at least 85%, at least 90%, at least 95%, at least 96%, at least 97%, at least 98%, at least 99%, or 100%) amino acid sequence identity to SEQ ID NO:18 and an immunoglobulin light chain variable domain (V_{L}) comprising an amino acid sequence having at least about 70% amino acid sequence identity (e.g., at least 75%, at least 80%, at least 85%, at least 90%, at least 95%, at least 96%, at least 97%, at least 98%, at least 99%, or 100%) amino acid sequence identity to SEQ ID NO:37. In some example embodiments, the polypeptide comprises an immunoglobulin heavy chain variable domain (V_{H}) comprising an amino acid sequence having at least 90% amino acid sequence identity to SEQ ID NO:18 and an immunoglobulin light chain variable domain (V_{L}) comprising an amino acid sequence having at least 90% amino acid sequence identity to SEQ ID NO:37. In some example embodiments, the polypeptide comprises an immunoglobulin heavy chain variable domain (V_{H}) comprising an amino acid sequence having at least 95% amino acid sequence identity to SEQ ID NO:18 and an immunoglobulin light chain variable domain (V_{L}) comprising an amino acid sequence having at least 95% amino acid sequence identity to SEQ ID NO:37.

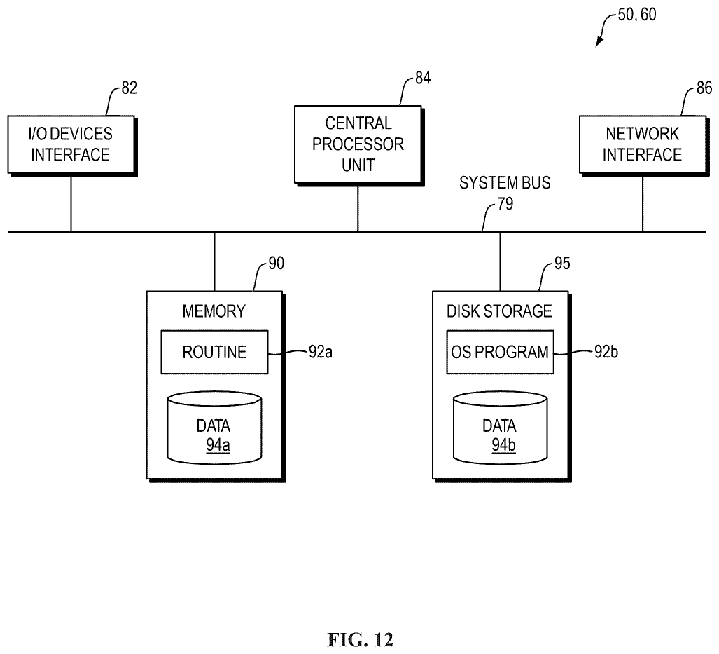

As noted, the method 1000 is computer-implemented and, as such, the functionality and effective operations, e.g., the determining, generating, and determining (1001) and generating (1002), are automatically implemented by one or more digital processors. The method 1000 can also be implemented using any computer device or combination of computing devices known in the art. Among other examples, the method 1000 can be implemented using computer(s)/device(s) 50 and/or 60 described hereinbelow in relation to FIGS. 11 and 12.

In an example embodiment of the method 1000, a given plurality of energy scores determined at step 1001 may include at least one of: a single amino acid energy score and a pairwise amino acid energy score.

According to an example embodiment of the method 1000, for at least one amino acid position of the amino acid sequence of the polypeptide, determining the plurality of energy scores at step 1001 may be further based on having substituted the amino acid at the amino acid position in the amino acid sequence with each of a plurality of different amino acids.

In an example embodiment of the method 1000, for at least one amino acid position of the amino acid sequence of the polypeptide, generating the partition function at step 1001 may be further based on a softmax function.

According to an example embodiment of the method 1000, at least one of: (1) predicting the functional property of the polypeptide may be implementable by a script of Appendix A or Appendix B, and (2) the CBO model may be substantially similar to a table of Appendix C.

In an example embodiment of the method 1000, the generated analysis score at step 1002 may be above a threshold. The threshold may be a score from the CBO model of one or more of a reference polypeptide that includes a V_{H} and V_{L} pair selected from:
SEQ ID NO:5 and SEQ ID NO:24 (Reference);
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:18 and SEQ ID NO:37 (AB-13);
SEQ ID NO:19 and SEQ ID NO:38 (AB-14c);
SEQ ID NO:20 and SEQ ID NO:39 (AB-15c); or
a combination of any of the foregoing.

According to an example embodiment of the method 1000, the functional property predicted at step 1002 may be a binding affinity for interleukin-4 receptor alpha (IL-4Rα).

In an example embodiment of the method 1000, the functional property predicted at step 1002 may be at least one of:
a binding affinity for interleukin-4 receptor alpha (IL-4Rα) characterized by a K_{D} of about 1 μM or less,
a binding affinity for IL-4Rα characterized by a kₐ of about 100×10⁵ M⁻¹ s⁻¹ or less,
a dissociation from IL-4Rα characterized by a k_{d} of about 100×10⁻⁵ s⁻¹ or less,
a binding affinity for IL-4Rα characterized by an EC₅₀ of about 1 μM or less,
a blocking activity against IL-4 Type II signaling characterized by an IC₅₀ of about 1 μM or less,
a blocking activity against IL-13 Type II signaling characterized by an IC₅₀ of about 1 μM or less, and
an inhibitory activity against CD23 expression in B cells characterized by an IC₅₀ of about 1 μM or less.

According to an example embodiment of the method 1000, the functional property predicted at step 1002 may be at least one of:
a binding affinity for interleukin-4 receptor alpha (IL-4Rα) characterized by a K_{D} of about 0.05 to 0.5 nM or less,
a binding affinity for IL-4Rα characterized by a kₐ of about 7×10⁵ to 9×10⁵ M⁻¹ s⁻¹ or less,
a dissociation from IL-4Rα characterized by a k_{d} of about 2×10⁻⁵ to 3×10⁻⁵ s⁻¹ or less,
a binding affinity for IL-4Rα characterized by an EC₅₀ of about 0.02 to 1.5 nM or less,
a blocking activity against IL-4 Type II signaling characterized by an IC₅₀ of about 1.1 to 7.3 nM or less,
a blocking activity against IL-13 Type II signaling characterized by an IC₅₀ of about 3 to 3.3 nM or less, and
an inhibitory activity against CD23 expression in B cells characterized by an IC₅₀ of about 8 to 50 nM or less.

In an example embodiment of the method 1000, the functional property predicted at step 1002 may be at least one of:
a binding affinity for interleukin-4 receptor alpha (IL-4Rα) characterized by a K_{D} of about 0.02 to 0.04 nM or less,
a binding affinity for IL-4Rα characterized by a kₐ of about 7.7×10⁵ to 8.9×10⁵ M⁻¹ s⁻¹ or less,
a dissociation from IL-4Rα characterized by a k_{d} of about 2.1×10⁵ to 2.6×10⁻⁵ s⁻¹ or less,
a binding affinity for IL-4Rα characterized by an EC₅₀ of about 0.04 to 1.3 nM or less,
a blocking activity against IL-4 Type II signaling characterized by an IC₅₀ of about 4.4 to 7.3 nM or less, and
an inhibitory activity against CD23 expression in B cells characterized by an IC₅₀ of about 30 to 50 nM or less.

According to an example embodiment of the method 1000, the functional property predicted at step 1002 may be at least one of:
a blocking activity against IL-4 Type II signaling characterized by an IC₅₀ of about 1.1 to 1.8 nM or less, and an inhibitory activity against CD23 expression in B cells characterized by an IC₅₀ of about 30 to 34 nM or less.

In an example embodiment of the method 1000, the functional property predicted at step 1002 may be an inhibitory activity against CD23 expression in B cells characterized by an IC₅₀ of about 8 to 8.8 nM or less.

According to an example embodiment of the method 1000, the functional property predicted at step 1002 may relate to modulating activity of a target molecule. The target molecule may be interleukin-4 receptor alpha (IL-4Rα).

In an example embodiment of the method 1000, the analysis score generated at step 1002 may be a binding score.

According to an example embodiment of the method 1000, the functional property predicted at step 1002 may be a binding affinity.

In an example embodiment of the method 1000, the polypeptide may not comprise a heavy chain having an amino acid sequence that is identical to SEQ ID NO:104 and a light chain having an amino acid sequence that is identical to SEQ ID NO:161.

Embodiments, e.g., the method 1000, can be used as part of a design or development process. For instance, embodiments can be employed as part of an iterative design process where functional properties of candidate polypeptide sequences are evaluated. Based on results of the evaluation, e.g., a given candidate polypeptide sequence meeting a standard, a real-world polypeptide may be manufactured based on the polypeptide sequence that met the standard. In such an embodiment, a given polypeptide sequence may be automatically transmitted to a manufacturing device and the manufacturing device may responsively and automatically generate the real-world polypeptide.

### Computer Support

FIG. 11 is a schematic view of a computer network in which embodiments may be implemented. Client computer(s)/devices 50 and server computer(s) 60 provide processing, storage, and input/output (I/O) devices executing application programs and the like. Client computer(s)/device(s) 50 can also be linked through communications network 70 to other computing devices, including other client device(s)/processor(s) 50 and server computer(s) 60. The communications network 70 can be part of a remote access network, a global network (e.g., the Internet), cloud computing servers or service, a worldwide collection of computers, local area or wide area networks, and gateways that currently use respective protocols (e.g., TCP/IP, Bluetooth®, etc.) to communicate with one another. Other electronic device/computer network architectures are also suitable.

FIG. 12 is a block diagram illustrating an example embodiment of a computer node (e.g., client processor(s)/device(s) 50 or server computer(s) 60) in the computer network 70 of FIG. 11. Each computer node 50, 60 contains system bus 79, where a bus is a set of hardware lines used for data transfer among components of a computer or processing system. The system bus 79 is essentially a shared conduit that connects different elements of a computer system (e.g., processor, disk storage, memory, I/O ports, network ports, etc.) that enables transfer of information between the elements. Attached to the system bus 79 is an I/O devices interface 82 for connecting various input and output devices (e.g., keyboard, mouse, display(s), printer(s), speaker(s), etc.) to the computer node 50, 60. A network interface 86 allows the computer node to connect to various other devices attached to a network (e.g., the network 70 of FIG. 11). A memory 90 provides volatile storage for computer software instructions 92a and data 94a used to implement some embodiments of the present disclosure (e.g., the method 1000 of FIG. 10, etc.). A disk storage 95 provides non-volatile storage for the computer software instructions 92b and data 94b used to implement some embodiments of the present disclosure. A central processor unit 84 is also attached to the system bus 79 and provides for execution of computer instructions.

In an embodiment, the processor routines 92a-92b and data 94a-94b are a computer program product (generally referenced as 92), including a non-transitory, computer readable medium (e.g., a removable storage medium such as DVD-ROM(s), CD-ROM(s), diskette(s), tape(s), etc.) that provides at least a portion of the software instructions for the disclosure system. The computer program product 92 can be installed by any suitable software installation procedure, as is well known in the art. In another embodiment, at least a portion of the software instructions may also be downloaded over a cable, communication, and/or wireless connection. In other embodiments, the disclosure programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present disclosure routines/program 92.

In alternative embodiments, the propagated signal is an analog carrier wave or digital signal carried on the propagated medium. For example, the propagated signal may be a digitized signal propagated over a global network (e.g., the Internet), a telecommunications network, or other networks (such as the network 70 of FIG. 11). In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of the computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium, and the like.

In other embodiments, the program product 92 may be implemented as a so-called Software as a Service (SaaS), or other installation or communication supporting end-users.

Embodiments or aspects thereof may be implemented in the form of hardware including but not limited to hardware circuitry, firmware, or software. If implemented in software, the software may be stored on any non-transient computer readable medium that is configured to enable a processor to load the software or subsets of instructions thereof. The processor then executes the instructions and is configured to operate or cause an apparatus to operate in a manner as described herein.

Further, hardware, firmware, software, routines, or instructions may be described herein as performing certain actions and/or functions of the data processors. However, it should be appreciated that such descriptions contained herein are merely for convenience and that such actions in fact result from computing devices, processors, controllers, or other devices executing the firmware, software, routines, instructions, etc.

It should be understood that the flow diagrams, block diagrams, and network diagrams may include more or fewer elements, be arranged differently, or be represented differently. But it further should be understood that certain implementations may dictate the block and network diagrams and the number of block and network diagrams illustrating the execution of the embodiments be implemented in a particular way.

Accordingly, further embodiments may also be implemented in a variety of computer architectures, physical, virtual, cloud computers, and/or some combination thereof, and, thus, the data processors described herein are intended for purposes of illustration only and not as a limitation of the embodiments.

### EXEMPLIFICATION

### Example 1. Generation and Characterization of IL-4Rα-Binding Polypeptides (Overview)

### Diversity Generation Campaign

The diversity generation campaign chose a human anti-IL-4Rα antibody as a Reference Antibody (“Reference”). A variant set of 384 antibodies was generated, and data on target binding (surface plasmon resonance or “SPR”) was acquired. A subset of 205 target-binding antibodies was selected and tested for developability and function. Two of the 205 target-binding antibodies bound IL-4Rα with high affinity and were selected as seeds for the co-optimization campaign.

### Co-Optimization Campaign

Using seeds from the diversity generation campaign, a second variant set of 384 antibodies was generated in the project learning and co-optimization campaign, and data on target binding was acquired. A subset of 163 target-binding antibodies was selected and tested for developability and function. Developability and function data were combined to identify 20 screening hits with acceptable developability parameters (Affinity-Capture Self-Interaction Nanoparticle Spectroscopy (AC-SINS)<10% shift, size-exclusion chromatography (SEC or aSEC)>85% monomer, a poly-specificity reagent binding via dissociation-enhanced lanthanide fluorescence immunoassay (PSR-DELFIA or PSR)<10 fold-change, hydrophobic interaction chromatography (HIC or aHIC)<10 minutes retention time) and similar binding kinetics relative to the Reference Antibody.

### Affinity Maturation Campaign

A set of 164 variants was screened of which 16 were further selected for functional screening. No variants exhibited improved functionality.

### Example 2. Binding Assays

### Surface Plasmon Resonance (SPR)

Surface plasmon resonance (SPR) was employed to determine the kinetics and affinities of binding of a panel of interleukin 4 receptor subunit alpha (IL-4Rα) antibodies to human IL-4Rα. The Carterra LSA and Cytiva Biacore 8K+instruments are label-free platforms that use SPR to measure binding interactions in real-time. Kinetic and affinity parameters were extracted from experimental data by an iterative process that found the best fit for a set of equations describing the interaction. The association rate constant kd (M⁻¹ s⁻¹) describes the rate at which a complex was formed. The dissociation rate constant kd (s⁻¹) describes the rate at which a complex dissociated. The equilibrium dissociation constant K_{D} (M) describes the strength of the interaction.

### Carterra LSA Non-Regenerative Capture Kinetics

To evaluate the affinities of binding of a panel of anti-IL-4Rα antibodies to human IL-4Rα at 25° C., a non-regenerative capture kinetics approach was performed using a Carterra LSA instrument (Carterra, Salt Lake City, UT). A goat anti-human polyclonal antibody lawn was prepared via amine-coupling onto a polycarboxylate hydrogel (HC30M) sensor chip using a single flow cell (SFC) to attain approximately 5,000 response units (RU). Each antibody was prepared at 8 μg/mL and captured onto regions of interest (ROI) for 15 min using four serial dockings of a 96-channel printhead (96PH) to create a 384-antibody array. Human IL-4Rα was prepared in HBSTE+BSA (10 mM HEPES pH 7.4, 150 mM NaCl, 3 mM EDTA, 0.01% Tween 20, 0.5 mg/mL BSA) running buffer and injected over the captured antibody array surface at nine concentrations in a 3-fold serial dilution series from 1000 nM to 0.2 nM. Each analyte concentration was injected sequentially from low to high concentration in a single cycle using the SFC with a 5-min association phase and a 10-min dissociation phase. The surface was regenerated by injecting two pulses of 0.85% phosphoric acid for 15 seconds between each cycle. The assay was performed at 25° C. with technical replicates. Kinetic parameters for the concentration series were obtained by double referencing and globally fitting the data to a 1:1 binding model with the 5% k_{d} option selected using the Kinetics analysis software (Carterra). The affinities of human IL-4Rα binding to a panel of IL-4Rα antibodies were reported, and the results were expressed as the average±standard deviation of one independent experiment with technical replicates (Table 7).

TABLE 7
Carterra LSA Non-regenerative Capture Kinetics.

| Protein | K_{D} (nM) | Type |
|---|---|---|
| AB-1c | not tested | hIgG4 WT |
| AB-2c | 50.68 ± 4.74 | hIgG4 WT |
| AB-3c | not tested | hIgG4 WT |
| AB-4c | 0.56 ± 0.03 | hIgG4 WT |
| AB-5c | 2.37 ± 0.17 | hIgG4 WT |
| AB-6c | ≤0.10 ± 0.00 | hIgG4 WT |
| AB-7c | 0.30 ± 0.01 | hIgG4 WT |
| AB-8c | 0.85 ± 0.14 | hIgG4 WT |
| AB-9c | 0.63 ± 0.03 | hIgG4 WT |
| AB-10c | 545.76 ± 18.47 | hIgG4 WT |
| AB-11c | 0.83 ± 0.11 | hIgG4 WT |
| AB-12c | ≤0.09 ± 0.01 | hIgG4 WT |
| AB-13c | ≤0.12 ± 0.01 | hIgG4 WT |

### Biacore 8K+Multi-Cycle Capture Kinetics

To evaluate the kinetics of bindings of select anti-IL-4Rα antibodies to human IL-4Rα at 25° C., a multi-cycle capture kinetics approach was implemented using a Biacore 8K+instrument (Cytiva, Marlborough, MA). A goat anti-human polyclonal antibody surface was prepared via amine-coupling onto a carboxymethylated dextran (CM4) sensor chip to attain approximately 5,000 response units (RU). Each antibody was prepared at 2-9 ug/mL and captured for 30 s at a flow rate of 10 μL min⁻¹ to achieve a capture level of approximately 150 RU. Human IL-4Rα was prepared in HBS-EP+ (10 mM HEPES pH 7.4, 150 mM NaCl, 3 mM EDTA, and 0.05% v/v surfactant P20) running buffer and injected over the captured antibody surface at six concentrations in a 3-fold serial dilution series from 30 nM to 0.1 nM. Each analyte concentration was injected in a separate cycle for 180 seconds at a flow rate of 30 μL min-1, and the complex was allowed to dissociate for 3,600 seconds. The surface was regenerated by injecting two pulses of 0.85% phosphoric acid for 20 seconds at a flow rate of 30 μL min-1 in between each cycle. The assay was performed at 25° C. with technical replicates. Kinetic parameters for the concentration series were obtained by double referencing and globally fitting the data to a 1:1 binding model using the Biacore Insight Evaluation software (Cytiva). The kinetics and affinities of human IL-4Rα binding to select IL-4Rα antibodies were reported, and results were expressed from a global fit of replicate data of one independent experiment (Table 8).

TABLE 8
Multi-Cycle Kinetics with Sample as Ligand (Biacore 8K+).

| | kₐ | k_{d} | K_{D} | |
|---|---|---|---|---|
| Protein | (M⁻¹s⁻¹) | (s⁻¹) | (pM) | Type |
| AB-13a | 7.78E+05 | 2.59E−05 | 33.3 | hIgG4 YTE |
| AB-13b | 8.81E+05 | 2.19E−05 | 24.9 | hIgG4 LS |
| AB-13c | 8.36E+05 | 2.22E−05 | 26.6 | hIgG4 |
| AB-13d | 7.89E+05 | 2.57E−05 | 32.6 | hIgG1 |

LS = IgG4 LS variant
YTE = IgG4 YTE variant

### Example 3. Functional Assays

### Human IL-4Rα Binding Assay (Indirect ELISA)

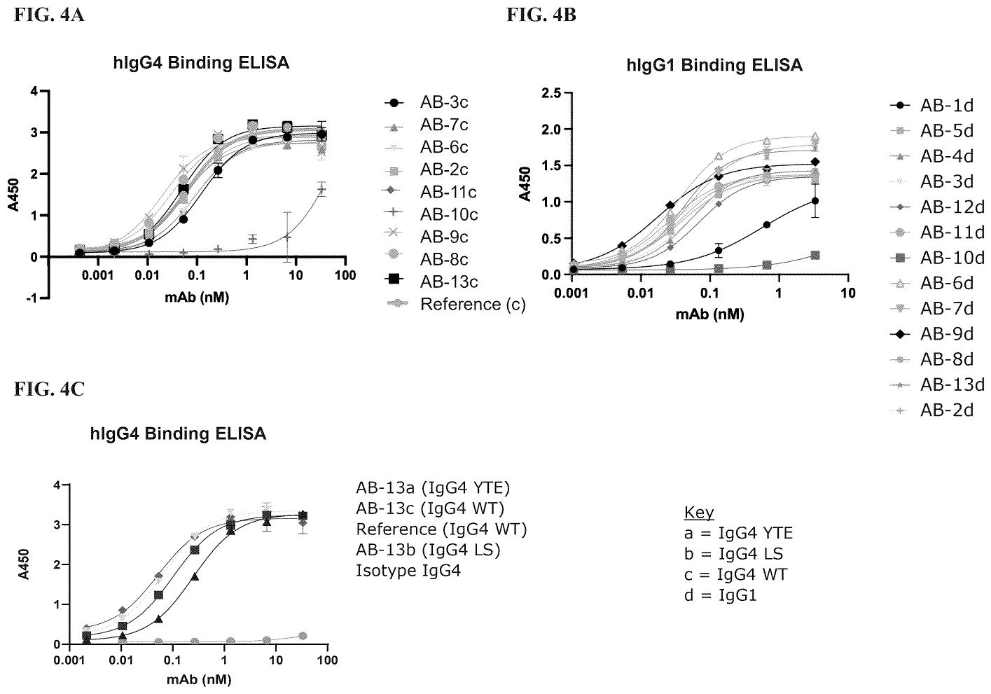

Binding affinity of antibodies to human IL-4Rα was assessed using an indirect enzyme-linked immunosorbent assay (ELISA). A 96-well half-area plate was coated with 2 μg/mL of recombinant human IL-4Rα (BioLegend, San Diego, CA) overnight. The next day, the plate was washed three times with ELISA Wash Buffer (phosphate-buffered saline (PBS)-Tween 0.5%), blocked with 1×ELISA diluent (BioLegend) for one hour. The plates were then washed three times with ELISA Wash Buffer, followed by addition of the anti-IL-4Rα antibodies (1:5 serial dilution 8-point curve starting at 5 ug/mL antibody) and incubated at 37° C. for one hour. The plate was washed three times with ELISA Wash Buffer. Horseradish peroxidase (HRP)-conjugated anti-human immunoglobulin G fragment crystallizable region (IgG Fc) secondary antibody (Promega Corporation, Madison, WI) at 1:2000 dilution in 1×ELISA diluent was added to detect binding of the primary antibody to the antigen. The plate was incubated for 30 minutes at 37° C. with the secondary detection antibody, then the plate was washed and developed with 3,3′, 5,5′-tetramethylbenzidine (TMB) substrate solution (Thermo Fisher Scientific, Waltham, MA). The HRP enzymatic reaction was stopped after about 1 minute with acidic stop solution (Thermo Fisher Scientific), and the absorbance at 450 nm (A450) was determined using a standard plate reader (FIGS. 4A-4C).

### HEK-Blue Blocking Assay

The ability of an antibody to block binding of IL-4/13 to IL-4Rα was assessed using the HEK (human embryonic kidney)-Blue colorimetric assay. HEK-Blue cells are an engineered cell line developed by InvivoGen (San Diego, CA); these modified HEK cells have the gene for secreted alkaline phosphatase (SEAP) inserted within the locus activated by Signal Transducer and Activator of Transcription 6 (STAT6) downstream of IL-4/IL-4Rα binding. When SEAP interacts with InvivoGen's QUANTI-Blue solution, a reaction turns the liquid from pink to blue.

For this assay, HEK-Blue cells were grown to confluency in a media comprising DMEM+GLUTAMAX® (Gibco, Waltham, MA), 10% fetal bovine serum (FBS), 1% Penicillin-Streptomycin (Gibco), and 100 μg/mL Normocin (InvivoGen). Cells were removed from their flasks using 0.25% trypsin-EDTA (Gibco), washed in media, and replated in a 96-well flat-bottom plate at 1 million cells per milliliter, and 100 μl per well. The cells were then blocked with the anti-IL-4Rα antibody at a concentration curve for thirty minutes at 37° C. Afterwards, they were incubated overnight with 100 μl of 5 ng/mL human IL-4/13 cytokine (ACROBiosystems, Newark, DE).

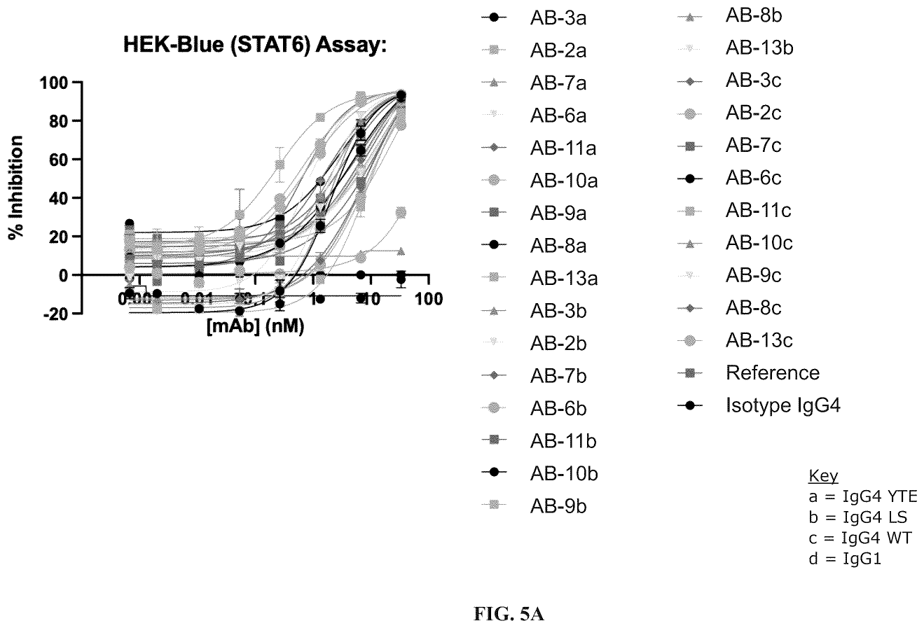

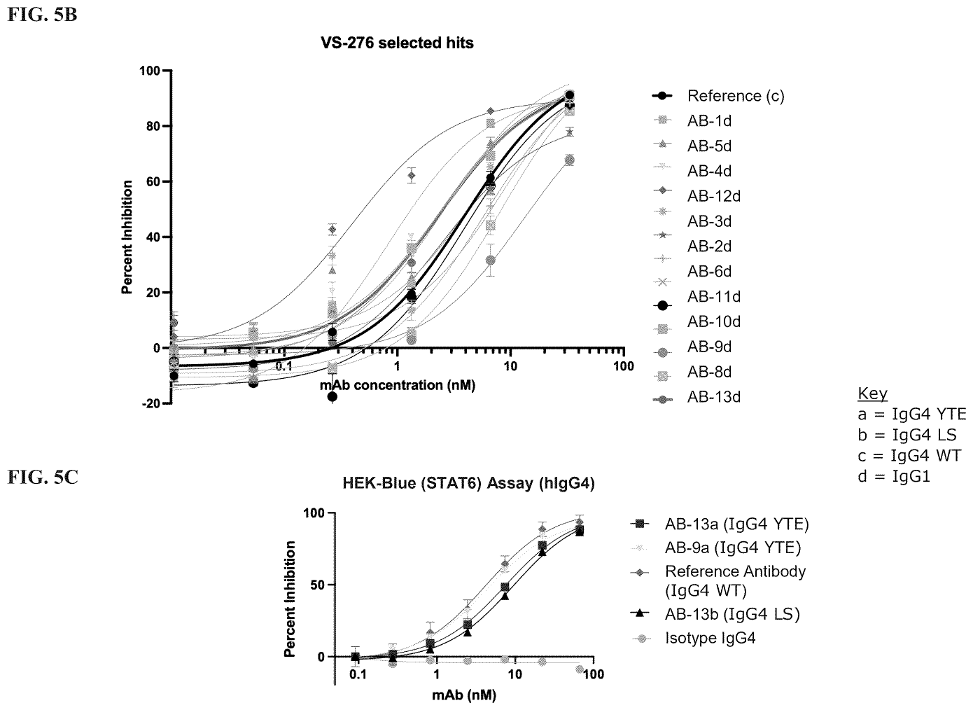

The next day, 20 μl of cell supernatant was extracted from each well and mixed with 180 μl of QUANTI-Blue solution (InvivoGen). This mixture was then allowed to develop in the dark for thirty minutes at room temperature. After developing, the absorbance was measured on a plate reader at 635 nm, indicating the amount of SEAP produced and thus the level of IL-4/IL-4Rα binding in the cells despite antibody blocking (FIGS. 5A-5C).

### IL-4Rα Human Primary B Cell-Based Assay (CD23 Inhibition)

Frozen peripheral blood mononuclear cells (PBMCs) from Donors were thawed, and B cells were isolated from frozen cell samples using the B cell isolation kit from STEMCELL Technologies (Vancouver, Canada; Cat. No. 17954) following the manufacturer's instructions. A sample from each donor was taken and stained using the B cell flow panel listed in the table below to check purity of the B cell population.

| Reagent/Material | Vendor | Catalog No. |
|---|---|---|
| Human TruStain FcX (Fc Block) | BioLegend | 422302 |
| PerCP-Cy5.5 Mouse Anti-Human CD19 | BD* | 561295 |
| PE Mouse Anti-Human CD23 | BD | 555711 |
| Brilliant Violet 421 anti-human IL-4Rα | BioLegend | 355014 |
| APC anti-human common γ chain | BioLegend | 338608 |
| FITC Mouse Anti-Human CD27 | BD | 555440 |
| Zombie NIR Fixable Viability Dye | BioLegend | 423106 |
| Control samples: | | |
| Isotype controls for flow cytometry | | |
| Unstained cells | | |
| L/D control | | |

*Becton, Dickinson and Company (BD), Franklin Lakes, NJ

After isolation, B cells were plated at 3×10⁵ ells in 100 μL C10 media in a 96 well U-bottom plate and rested overnight at 37° C. The following day, anti-IL-4Rα antibody was added in 50 μL (3-fold 10-point curve starting at 150 μg/mL antibody) and incubated for 30 minutes at 37° C. IL-4 cytokine (BioLegend Cat. No. 574006) was added in 50 μL of media for controls to a final concentration of cytokine of 10 ng/mL in the assay. Total assay volume was 200 μL and was incubated for 48 hours. After 48 hours, the cells were stained according to the staining protocol in the table below to identify CD23 inhibition by anti-IL-4Rα antibodies.

| Step | Protocol |
|---|---|
| 1 | After incubation, add 200 μL of 1X PBS to wash cells, centrifuge cells at 300 x g for 5 |
| | minutes at room temperature (RT) and decant the supernatant |
| 2 | Resuspend cells in 100 μL of viability dye in PBS |
| 3 | Incubate 10 mins at RT protected from light |
| 4 | Wash once with 1X PBS (centrifuge cells at 300 x g for 5 minutes at RT and decant the |
| | supernatant) |
| 5 | Resuspend with 5 μL/well of Fc Block |
| 6 | After 5 mins add 100 μL of surface Ab mix in FACS buffer. Stain isotypes and controls |
| | separately, 5 isotype controls for flow + unstained cells + L/D control |
| 7 | Mix and incubate for 25 mins at 4° C. protected from light |
| 8 | Wash once with FACS buffer (centrifuge cells at 300 x g for 5 minutes at RT and |
| | decant the supernatant) |
| 9 | Resuspend in 100 μL of FixPerm buffer to fix cells |
| 10 | Wash once with FACS buffer and resuspend in 200 μL of FACS buffer for analysis |
| 11 | Run samples on the Attune flow cytometer checking for CD23 expression on CD19⁺ B |
| | cells |

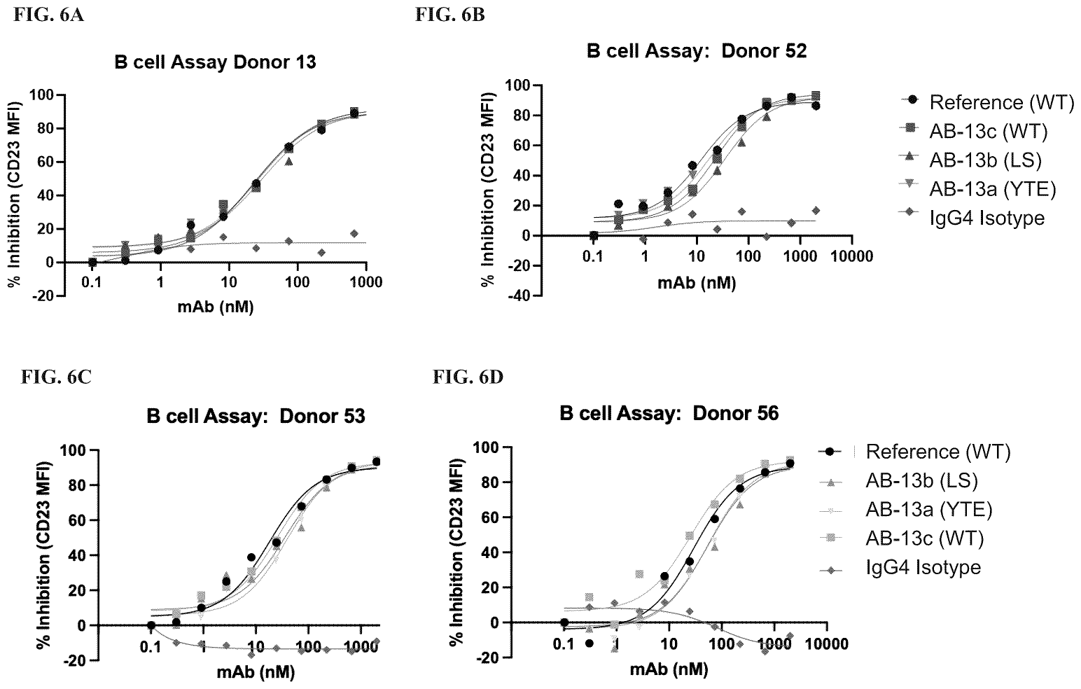

Inhibition of CD23 expression was reported from 4 donors as IC₅₀ (Table 9) and percent inhibition (FIGS. 6A-6D).

TABLE 9
Inhibition of CD23 Expression by Primary B cells (IC₅₀)

| | Donor 13 | Donor 52 | Donor 53 | Donor 56 | |
|---|---|---|---|---|---|
| Protein | IC₅₀ (nM) | IC₅₀ (nM) | IC₅₀ (nM) | IC₅₀ (nM) | Type |
| Reference | 22.03 | 11.93 | 18.04 | 29.76 | hIgG4 |
| AB-13a | 27.49 | 17.81 | 40.61 | 54.36 | hIgG4 YTE |
| AB-13b | 30.13 | 34.44 | 35.89 | 54.19 | hIgG4 LS |
| AB-13c | 24.92 | 23.04 | 26.17 | 25.81 | hIgG4 |

LS = IgG4 LS variant
YTE = IgG4 YTE variant

### Ramos CD23 Inhibition Assay

Ramos (RA1) cells (ATCC, Manassas, VA; Cat. No. CRL-1596) were harvested when the cell density was greater than 3×10⁶ cells/mL. Ramos cells were plated at 3×10⁵ cells in 100 μL RPMI 1640 with 10% FBS and 1% Pen Strep media in a 96 well U-bottom plate. Anti-IL-4Rα antibody was added in 50 μL (3-fold 8-point curve starting at 30 μg/mL antibody) and was incubated for 30 minutes at 37° C. 50 μL IL-4 cytokine (BioLegend Cat. No. 574006) was added for a final concentration of 10 ng/ml of cytokine in a total assay volume of 200 μL. In control experiments, 50 μL media was added. The assay was incubated for 24 hours. After 24 hours, the cells were stained according to the protocol set forth in the tables below to identify CD23 inhibition by anti-IL-4Rα antibodies.

| Reagent/Material | Vendor | Catalog No. |
|---|---|---|
| Human TruStain FcX (Fc Block) | BioLegend | 422302 |
| Brilliant Violet 421 anti-human IL-4Rα | BioLegend | 355014 |
| PE/Cy7 anti-human IL-13Rα1 | BioLegend | 360408 |
| APC anti-human common γ chain | BioLegend | 338608 |
| Zombie NIR Fixable Viability Dye | BioLegend | 423106 |
| FITC anti-human CD23 | BioLegend | 338506 |
| Control samples: | | |
| 5 isotype controls for flow | | |
| Unstained cells | | |

| Step | Protocol |
|---|---|
| 1 | After incubation, add 200 μL of 1X PBS to wash cells, centrifuge cells at 300 x g for 5 |
| | minutes at room temperature (RT) and decant the supernatant |
| 2 | Resuspend cells in 100 μL of viability dye in PBS |
| 3 | Incubate 10 mins at RT protected from light |
| 4 | Wash once with 1X PBS (centrifuge cells at 300 x g for 5 minutes at RT and decant the |
| | supernatant) |
| 5 | Resuspend with 5 μL/well of Fc Block |
| 6 | After 5 mins add 100 μL of surface Ab mix in FACS buffer. Stain isotypes and controls |
| | separately, 5 isotype controls for flow + unstained cells + L/D control |
| 7 | Mix and incubate for 25 mins at 4° C. protected from light |
| 8 | Wash once with FACS buffer (centrifuge cells at 300 x g for 5 minutes at RT and |
| | decant the supernatant) |
| 9 | Resuspend in 100 μL of FixPerm buffer to fix cells |
| 10 | Wash once with FACS buffer and resuspend in 200 μL of FACS buffer for analysis |
| 11 | Run samples on the Attune flow cytometer checking for CD23 expression on CD19⁺ B |
| | cells. |

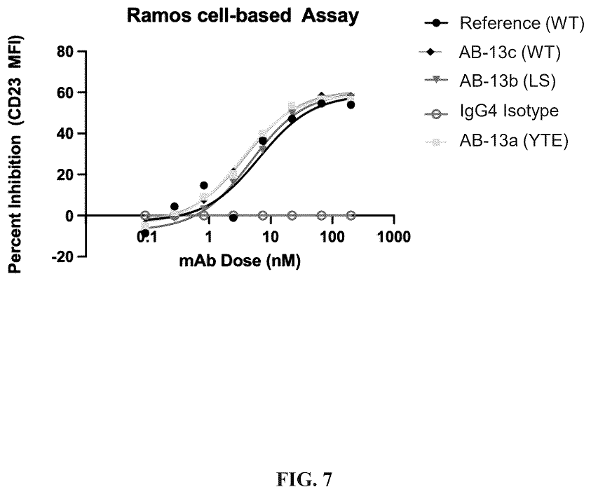

Results were reported as percent inhibition (FIG. 7).

### Example 4. Translational Assays

pSTAT6 Whole Blood Assay

The objective was to evaluate IL-4Rα target engagement in primary cells via functional inhibition of relevant atopic dermatitis (AD) biomarkers, such as phosphorylated Signal Transducer and Activator of Transcription 6 (pSTAT6), and Thymus- and Activation-Regulated Chemokine (TARC or CCL17). Assay development, optimization, and proof-of-concept were done using the Reference (anti-IL-4Rα mAb) in peripheral blood mononuclear cells (PBMCs) and ultimately transferred into whole blood across three distinct donors.

See tables below for a list of materials used in this assay.

| | | Emission | | Catalog | | |
|---|---|---|---|---|---|---|
| Reagent | Fluorophore | Filter | Vendor | No. | Clone | Isotype |
| Anti-CD3 | BV421 | 440/50 | BioLegend | 300433 | UCHT1 | mIgG1k |
| Anti-CD4 | FITC | 530/30 | BD | 300538 | RPA-T4 | mIgG1k |
| Anti-CD19 | APC | 670/14 | BioLegend | 302212 | H1B19 | mIgG1k |
| Anti- pSTAT6 | PE | 585/16 | BioLegend | 686004 | A15137E | mIgG1k |
| Fixable Viability | APC- | 780/60 | Invitrogen | 65-0865-14 | N/A | |
| Dye (FVD) | eFluor780 | | | | | |
| Human TruStain | N/A | N/A | BioLegend | 422301 | N/A | |
| FcX | | | | | | |

| Reagent | Vendor | Catalog No. |
|---|---|---|
| rh-IL-4 | R&D Systems* | 204-1L |
| BD Phosflow Lyse/Fix Buffer 5x | BD | 558049 |
| Flow Cytometry staining buffer | eBioscience** | 00-4222-26 |
| BD Phosflow Perm Buffer II | BD Biosciences*** | 558052 |

*R&D Systems, Minneapolis, MN
**eBioscience, Waltham, MA
***BD Biosciences, Franklin Lakes, NJ

A volume of 180 μL/well of whole blood was plated in RPMI-1% FBS, in a 96 well flat bottom plate. Appropriate concentration of mAb was added at 20× (in a volume of 10 μL). The plate was incubated at 37° C. for 20 minutes. Either 10 ng/mL or 1 ng/ml of rh-IL-4 was then added in 20× (volume of 10 μL). The plate was incubated at 10 minutes at 37° C. The plate was centrifuged, and the supernatant was aspirated to leave a volume of about 100 μL.

Stimulation was then stopped by addition of Lyse/Fix buffer (BD) at a volume of 20 times the remaining volume of the blood. The samples were vigorously mixed via pipetting and then incubated at 37° C. for 10-15 minutes during lysing of red blood cells (RBCs) and fixation. The cells were then washed twice in flow cytometry staining buffer and stained with fixable viability dye and FC block for 15 minutes at 4° C. Extracellular markers were added according to the concentrations suggested by manufacturer's instructions. The cells were then washed 2× with flow cytometry staining buffer before being resuspended in BD Phosflow Perm II buffer for 30 minutes. The cells were once again washed as described previously and resuspended in intracellular staining markers (pSTAT6) according to manufacturer's instructions for 30 minutes at room temperature. After final 2 washes, the cells were resuspended in flow cytometry staining buffer, run on Attune CytPix flow cytometer (Invitrogen), and analyzed using FlowJo software (FlowJo, Ashland, OR). The graphs of data exported from FlowJo were visualized and statistically analyzed using GraphPad Prism (GraphPad Software, Boston, MA).

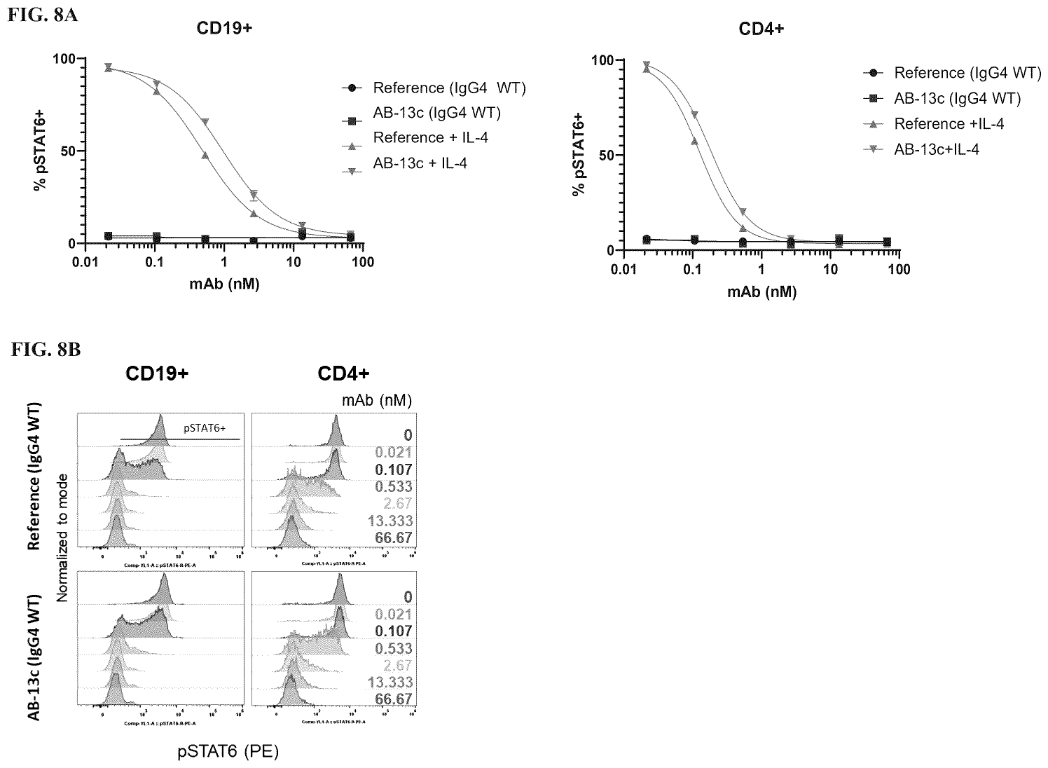

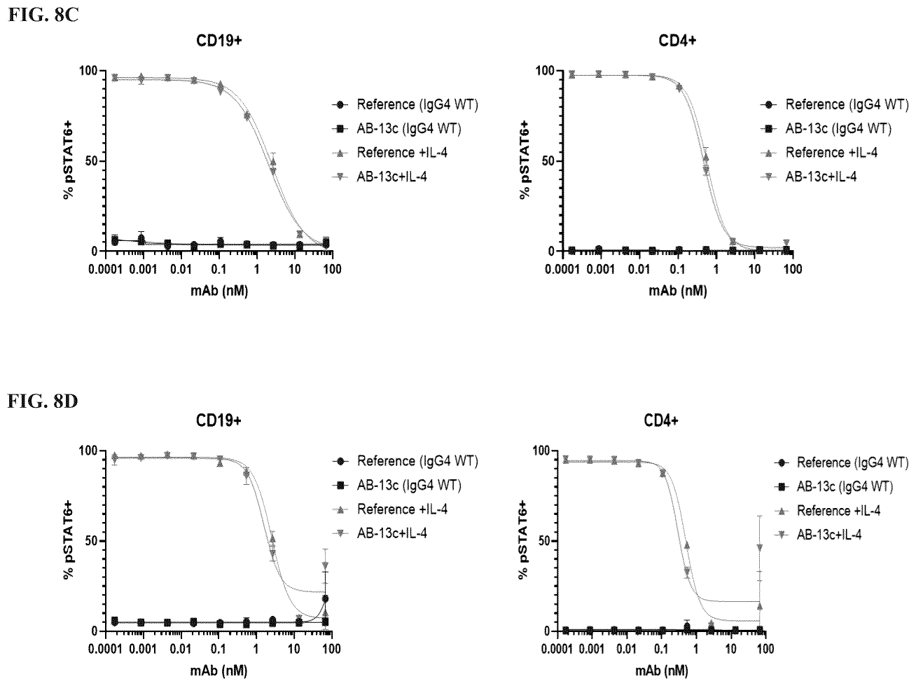

In the presence of IL-4, there was a dose-dependent decrease in pSTAT6 expression with both the Reference (hIgG4) and AB-13c (hIgG4) in both B cells (CD19+) and T cells (CD4+) (FIGS. 8A, 8B). These results were consistent with the data obtained from two additional whole blood donors (FIGS. 8C, 8D). The dose-dependent decreases in pSTAT6 were comparable between the Reference and AB-13c in whole blood. Minimal impact from the biological matrix of whole blood (matrix effect) was observed as evidenced by the ability of the respective antibodies to retain pSTAT6 modulation, indicative of functional inhibition of IL-4Rα.

TARC Release (from PBMCs) Assay

The objective was to evaluate IL-4Rα target engagement in primary cells via functional inhibition of relevant atopic dermatitis (AD) biomarkers, such as phosphorylated Signal Transducer and Activator of Transcription 6 (pSTAT6) and Thymus- and Activation-Regulated Chemokine (TARC or CCL17). TARC release can be used as a functional readout of anti-IL-4Rα activity via ELISA readout. Assay development, optimization and proof-of-concept experiments were done using the Reference (hIgG4 IL-4Rα mAb) in PBMCs. AB-13c (hIgG4) was tested in comparison to the Reference in one donor. The assay was optimized to be used in further potential uses of AB-13c.

See table below for a list of materials used in this assay.

| Reagent/Material | Vendor | Catalog No. |
|---|---|---|
| Recombinant Human IL-4 Protein | R&D Systems | 204-IL |
| (rh-IL-4) | | |
| Reference Antibody | | |
| AB-13c | | |
| Human CCL17/TARC Quantikine | R&D Systems | SDN00 |
| ELISA kit | | |

PBMCs were plated in a 96 well flat bottom plate at about 5×10⁴ to about 1×10⁵ cells per well in a volume of 50 μL of RPMI-10% FBS. An appropriate concentration of mAb was added at 2× (in a volume of 50 μL). The plate was incubated at 37° C. for 20 minutes. Zero, 1, or 10 ng/mL of rh-IL-4 was then added in 3× (volume of 50 μL). The plate was then incubated at 37° C. for 48-72 hours and then centrifuged. The supernatants were collected and frozen at −80° C. until use. Undiluted samples were run in accordance with manufacturer's instructions for the Human CCL17/TARC QUANTIKINE® ELISA kit (R&D Systems). Optical density (OD) of the samples were read at 450 nM on a spectrophotometer. The data was analyzed using a nonlinear regression curve and graphed using GraphPad Prism software.

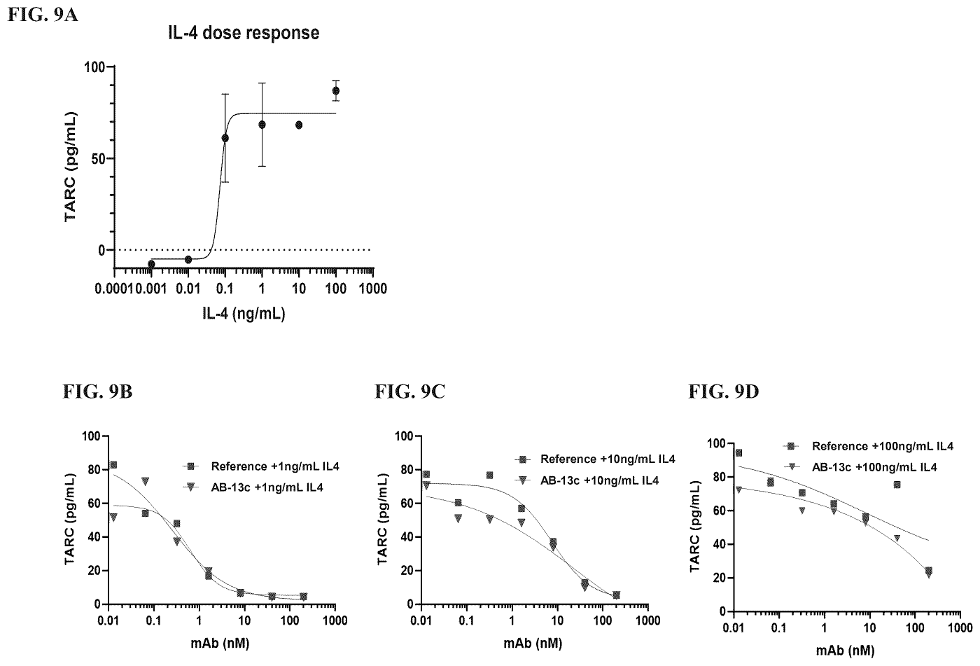

A dose-dependent induction of TARC release in response to rh-IL-4 stimulation was observed (FIG. 9A). An ideal range for IL-4 stimulation was from 0.1 to 100 ng/mL. At different levels of IL-4 stimulation (1, 10, and 100 ng/ml IL-4), AB-13c showed a degree of TARC suppression comparable to the Reference (FIGS. 9B-9D).

The teachings of all patents, published applications, and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A polypeptide comprising:
a) an immunoglobulin heavy chain variable domain (V_{H}) amino acid sequence comprising a heavy chain complementarity determining region 1 (HCDR1), a heavy chain complementarity determining region 2 (HCDR2), and a heavy chain complementarity determining region 3 (HCDR3) that are substantially similar to an HCDR1, HCDR2 and HCDR3, respectively, of the amino acid sequence of any one of SEQ ID NOs: 6-20; and
b) an immunoglobulin light chain variable domain (V_{L}) amino acid sequence comprising a light chain complementarity determining region 1 (LCDR1), a light chain complementarity determining region 2 (LCDR2), and a light chain complementarity determining region 3 (LCDR3) that are substantially similar to an LCDR1, LCDR2 and LCDR3, respectively, of the amino acid sequence of any one of SEQ ID NOs: 25-39.

2. The polypeptide of claim 1, comprising the HCDR1, HCDR2 and HCDR3, and LCDR1, LCDR2 and LCDR3, of an antibody comprising an amino acid sequence selected from:
SEQ ID NO:18 and SEQ ID NO:37 (AB-13);
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO:19 and SEQ ID NO:38 (AB-14c); or
SEQ ID NO:20 and SEQ ID NO:39 (AB-15c).

3. A polypeptide comprising a paratope that is substantially similar to a paratope of an antibody comprising a V_{H} and V_{L} pair selected from:
SEQ ID NO:18 and SEQ ID NO:37 (AB-13);
SEQ ID NO:6 and SEQ ID NO:25 (AB-1);
SEQ ID NO:7 and SEQ ID NO:26 (AB-2);
SEQ ID NO:8 and SEQ ID NO:27 (AB-3);
SEQ ID NO:9 and SEQ ID NO:28 (AB-4);
SEQ ID NO:10 and SEQ ID NO:29 (AB-5);
SEQ ID NO:11 and SEQ ID NO:30 (AB-6);
SEQ ID NO:12 and SEQ ID NO:31 (AB-7);
SEQ ID NO:13 and SEQ ID NO:32 (AB-8);
SEQ ID NO:14 and SEQ ID NO:33 (AB-9);
SEQ ID NO:15 and SEQ ID NO:34 (AB-10);
SEQ ID NO:16 and SEQ ID NO:35 (AB-11);
SEQ ID NO:17 and SEQ ID NO:36 (AB-12);
SEQ ID NO: 19 and SEQ ID NO:38 (AB-14c); or
SEQ ID NO:20 and SEQ ID NO:39 (AB-15c); or
a combination of any of the foregoing.

4.-5. (canceled)

6. A polypeptide comprising an immunoglobulin heavy chain variable domain (V_{H}) comprising the amino acid sequence of SEQ ID NO:4, wherein:
X₁ is T or N;
X₂ is R, S or K;
X₃ is G, S, D or T;
X₄ is N, T or K;
X₅ is T, K or I;
X₆ is K or R;
X₇ is R or A;
X₈ is L, W or Y;
X₉ is S or T;
X₁₀ is I, A, K, T, S or V;
X₁₁ is T, A, K, S or R;
X₁₂ is I, V, A or T;
X₁₃ is R or K;
X₁₄ is R, Y, E, I, N, S, V or K;
X₁₅ is Y, V or R; or
X₁₆ is V or I;
or any combination of the foregoing.

7.-9. (canceled)

10. The polypeptide of claim 1, wherein the V_{H} comprises a heavy chain complementarity determining region 1 (HCDR1), a heavy chain complementarity determining region 2 (HCDR2) and a heavy chain complementarity determining region 3 (HCDR3) that are identical in amino acid sequence to the HCDR1, HCDR2 and HCDR3, respectively, of any one of SEQ ID NOs: 6-20.

11. The polypeptide of claim 1, wherein the V_{L} comprises a light chain complementarity determining region 1 (LCDR1), light chain complementarity determining region 2 (LCDR2) and light chain complementarity determining region 3 (LCDR3) that are identical in amino acid sequence to the LCDR1, LCDR2 and LCDR3, respectively, of any one of SEQ ID NOs: 25-39.

12.-17. (canceled)

18. The polypeptide of claim 1, wherein:
a) the V_{H} comprises the amino acid sequence of SEQ ID NO: 6 and the V_{L} comprises the amino acid sequence of SEQ ID NO:25 (AB-1);
b) the V_{H} comprises the amino acid sequence of SEQ ID NO:7 and the V_{L} comprises the amino acid sequence of SEQ ID NO:26 (AB-2);
c) the V_{H} comprises the amino acid sequence of SEQ ID NO:8 and the V_{L} comprises the amino acid sequence of SEQ ID NO:27 (AB-3);
d) the V_{H} comprises the amino acid sequence of SEQ ID NO:9 and the V_{L} comprises the amino acid sequence of SEQ ID NO:28 (AB-4);
e) the V_{H} comprises the amino acid sequence of SEQ ID NO:10 and the V_{L} comprises the amino acid sequence of SEQ ID NO:29 (AB-5);
f) the V_{H} comprises the amino acid sequence of SEQ ID NO:11 and the V_{L} comprises the amino acid sequence of SEQ ID NO:30 (AB-6);
g) the V_{H} comprises the amino acid sequence of SEQ ID NO: 12 and the V_{L} comprises the amino acid sequence of SEQ ID NO:31 (AB-7);
h) the V_{H} comprises the amino acid sequence of SEQ ID NO:13 and the V_{L} comprises the amino acid sequence of SEQ ID NO:32 (AB-8);
i) the V_{H} comprises the amino acid sequence of SEQ ID NO:14 and the V_{L} comprises the amino acid sequence of SEQ ID NO:33 (AB-9);
j) the V_{H} comprises the amino acid sequence of SEQ ID NO:15 and the V_{L} comprises the amino acid sequence of SEQ ID NO:34 (AB-10);
k) the V_{H} comprises the amino acid sequence of SEQ ID NO:16 and the V_{L} comprises the amino acid sequence of SEQ ID NO:35 (AB-11);
l) the V_{H} comprises the amino acid sequence of SEQ ID NO:17 and the V_{L} comprises the amino acid sequence of SEQ ID NO:36 (AB-12);
m) the V_{H} comprises the amino acid sequence of SEQ ID NO:18 and the V_{L} comprises the amino acid sequence of SEQ ID NO:37 (AB-13);
n) the V_{H} comprises the amino acid sequence of SEQ ID NO:19 and the V_{L} comprises the amino acid sequence of SEQ ID NO:38 (AB-14c); or
o) the V_{H} comprises the amino acid sequence of SEQ ID NO:20 and the V_{L} comprises the amino acid sequence of SEQ ID NO:39 (AB-15c).

19.-32. (canceled)

33. The polypeptide of claim 1, wherein the V_{H} and V_{L} are humanized, contain human framework regions, or any combination of the foregoing.

34. The polypeptide of claim 1, wherein the polypeptide binds an interleukin-4 receptor alpha (IL-4Rα).

35. The polypeptide of claim 1, wherein the polypeptide is an antibody or an antigen-binding fragment thereof.

36. The polypeptide of claim 35, wherein the antigen binding fragment is selected from Fab, F(ab′)₂, Fab′, scFv, or Fv.

37. The polypeptide of claim 35, comprising an antibody heavy chain constant domain sequence, an antibody light chain constant domain sequence, or both an antibody heavy chain constant domain sequence and an antibody light chain constant domain sequence.

38.-41. (canceled)

42. The polypeptide of claim 1, wherein the polypeptide is conjugated to a heterologous moiety.

43.-54. (canceled)

55. A polynucleotide (e.g., DNA or RNA; linear or circular; optionally containing one or more modified nucleotides) comprising a sequence encoding the polypeptide of claim 1.

56. A vector (e.g., an expression vector, including a viral-delivery vector) comprising the polynucleotide of claim 55.

57. A host cell comprising the polynucleotide of claim 55.

58. A composition comprising the polypeptide of claim 1.

59. The composition of claim 58, comprising one or more pharmaceutical excipients, diluents, or carriers.

60. A method of treating a subject in need thereof, comprising administering an effective amount of the composition of claim 58 to the subject.

61. A method of reducing inflammation, a symptom of an inflammatory condition, or risk of developing an inflammatory condition in a cell of a subject, comprising contacting the cell with an effective amount of the composition of claim 58.

62.-68. (canceled)

69. A computer-implemented method for predicting a functional property of a polypeptide, the method comprising, via a computationally binding optimized (CBO) model:
for each amino acid position of an amino acid sequence of the polypeptide:
determining a plurality of energy scores based on the amino acid position in the amino acid sequence;
generating a partition function based on the plurality of energy scores determined; and
determining a cross-entropy metric based on (i) an amino acid at the amino acid position in the amino acid sequence, (ii) a maximum energy score of the plurality of energy scores determined, and (iii) the generated partition function; and
generating an analysis score of the polypeptide based on each cross-entropy metric determined, the analysis score indicating a predicted functional property of the polypeptide,
wherein the polypeptide comprises an immunoglobulin heavy chain variable domain (V_{H}) comprising an amino acid sequence having at least 90% sequence identity to SEQ ID NO:18 and an immunoglobulin light chain variable domain (V_{L}) comprising an amino acid sequence having at least 90% sequence identity to SEQ ID NO:37, and
wherein the polypeptide does not comprise a V_{H} comprising an amino acid sequence having 100% sequence identity to SEQ ID NO:5 and a V_{L} comprising an amino acid sequence having 100% sequence identity to SEQ ID NO:24.

70.-84. (canceled)

85. A computer-based system for predicting a functional property of a polypeptide, the computer-based system comprising:
a processor; and
a memory with computer code instructions stored thereon, the processor and the memory, with the computer code instructions, being configured to cause the computer-based system to:
implement a computationally binding optimized (CBO) model, the CBO model configured to:
for each amino acid position of an amino acid sequence of the polypeptide:
determine a plurality of energy scores based on the amino acid position in the amino acid sequence;
generate a partition function based on the plurality of energy scores determined; and
determine a cross-entropy metric based on (i) an amino acid at the amino acid position in the amino acid sequence, (ii) a maximum energy score of the plurality of energy scores determined, and (iii) the generated partition function; and
generate an analysis score of the polypeptide based on each cross-entropy metric determined, the analysis score indicating a predicted functional property of the polypeptide,
wherein the polypeptide comprises an immunoglobulin heavy chain variable domain (V_{H}) comprising an amino acid sequence having at least 90% sequence identity to SEQ ID NO:18 and an immunoglobulin light chain variable domain (V_{L}) comprising an amino acid sequence having at least 90% sequence identity to SEQ ID NO:37, and
wherein the polypeptide does not comprise a V_{H} comprising an amino acid sequence having 100% sequence identity to SEQ ID NO:5 and a V_{L} comprising an amino acid sequence having 100% sequence identity to SEQ ID NO:24.

86. A polypeptide that binds interleukin-4 receptor alpha (IL-4Rα):
wherein the polypeptide is assigned a score above a threshold by an analysis via a computationally binding optimized (CBO) model,
wherein the polypeptide comprises an immunoglobulin heavy chain variable domain (V_{H}) comprising an amino acid sequence having at least 90% sequence identity to SEQ ID NO:18 and an immunoglobulin light chain variable domain (V_{L}) comprising an amino acid sequence having at least 90% sequence identity to SEQ ID NO:37, and
wherein the polypeptide does not comprise a V_{H} comprising an amino acid sequence having 100% sequence identity to SEQ ID NO:5 and a V_{L} comprising an amino acid sequence having 100% sequence identity to SEQ ID NO:24.

87.-94. (canceled)

95. A polypeptide that binds human interleukin-4 receptor alpha (IL-4Rα), wherein the polypeptide is selected by a method comprising:
evaluating a plurality of candidate polypeptides using a computationally binding optimized (CBO) model by, for each candidate polypeptide of the plurality of candidate polypeptides:
for each amino acid position of an amino acid sequence of the candidate polypeptide:
determining a plurality of energy scores based on the amino acid position in the amino acid sequence;
generating a partition function based on the plurality of energy scores determined; and
determining a cross-entropy metric based on (i) an amino acid at the amino acid position in the amino acid sequence, (ii) a maximum energy score of the plurality of energy scores determined, and (iii) the generated partition function; and
generating an analysis score of the candidate polypeptide based on each cross-entropy metric determined, the analysis score indicating a functional property of the polypeptide's ability to bind to human IL-4Rα; and
selecting a given candidate polypeptide from among the plurality of candidate polypeptides based a result of the evaluating,
wherein the polypeptide comprises an immunoglobulin heavy chain variable domain (V_{H}) comprising an amino acid sequence having at least 90% sequence identity to SEQ ID NO:18 and an immunoglobulin light chain variable domain (V_{L}) comprising an amino acid sequence having at least 90% sequence identity to SEQ ID NO:37, and
wherein the polypeptide does not comprise a V_{H} comprising an amino acid sequence having 100% sequence identity to SEQ ID NO:5 and a V_{L} comprising an amino acid sequence having 100% sequence identity to SEQ ID NO:24.
